(19) United States
(12) Patent Application Publication
(10) Pub. No.: US 2020/0352976 A1
(43) Pub. Date: Nov. 12, 2020

(54) HYPOMETHYLATING AGENTS FOR USE IN TREATMENT OF CANCER

(71) Applicant: Otsuka Pharmaceutical Co., Ltd, Tokyo (JP)

(72) Inventors: Mohammad AZAB, San Francisco, CA (US); Yong HAO, Dublin, CA (US); Harold KEER, Emerald Hills, CA (US)

(21) Appl. No.: 16/866,716

(22) Filed: May 5, 2020

Related U.S. Application Data
(60) Provisional application No. 62/843,892, filed on May 6, 2019.

(51) Int. Cl. A61K 31/7084; A61K 9/00; A61P 35/02

(52) U.S. Cl. CPC A61K 31/7084; A61P 35/02; A61K 9/0019

(57) ABSTRACT

Disclosed herein is a method for treating cancer with DNA-hypomethylating agents. Also disclosed herein is a method of selecting a treatment for a subject. Treatment can be given to a subject for more than one cycle of treatment to obtain improved efficacy of the treatment.

### CROSS-REFERENCE

The application claims the benefit of U.S. Provisional Application No. 62/843,892, filed May 6, 2019, which is incorporated by reference herein in its entirety.

### BACKGROUND

DNA methylation is a post-replicative chemical modification of DNA. Different cancers can be stratified by abnormal DNA methylation profiles (degree of global or specific DNA methylation), and the hypermethylation of specific genes can be associated with the prognosis of cancer. DNA methylation patterns can also be used to predict response or resistance to cancer therapy.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the invention provides a method of treating cancer in a subject in need thereof, the method comprising: (a) administering to the subject a therapeutic regimen, wherein the therapeutic regimen comprises administration of a therapeutically-effective amount of a DNA-hypomethylating agent once per day on days 1-5 of a treatment cycle, wherein the treatment cycle lasts 28 days; and (b) repeating the therapeutic regimen at least 3 times.

In some embodiments, the invention provides a method of treating cancer in a subject in need thereof, the method comprising administering to the subject a therapeutically effective amount of a DNA-hypomethylating agent on days 1-5 of a 28-day treatment cycle, wherein the therapeutically effective amount of the DNA-hypomethylating agent is about 60 mg per m² of body surface area of the subject, and wherein, in a controlled study:—each human of a group of humans has cancer;—60 mg per m² of body surface area of the DNA-hypomethylating agent is administered to each human of the group of humans on days 1-5 of a 28-day study treatment cycle; and—the group of humans has a median survival of about 408 to about 771 days.

In some embodiments, the invention provides a method of treating cancer in a subject in need thereof, the method comprising administering to the a therapeutically effective amount of a DNA-hypomethylating agent on days 1-5 of a 28-day treatment cycle, wherein the therapeutically effective amount of the DNA-hypomethylating agent is about 90 mg per m² of body surface area of the subject, and wherein, in a controlled study:—each human of a group of humans has cancer;—90 mg per m² of body surface area of the DNA-hypomethylating agent is administered to each human of the group of humans on days 1-5 of a 28-day study treatment cycle; and—the group of humans has a median survival of about 303 to about 663 days.

In some embodiments, the invention provides a method of treating cancer in a subject in need thereof, the method comprising administering to the subject a therapeutically effective amount of a DNA-hypomethylating agent on days 1-5 of a 28-day treatment cycle, wherein the therapeutically effective amount of the DNA-hypomethylating agent is about 60 mg per m² of body surface area of the subject, and wherein, in a controlled study of a group of humans:—each human of the group of humans has cancer;—60 mg per m² of body surface area of the DNA-hypomethylating agent is administered to each human of the group of humans on days 1-5 of a 28-day study treatment cycle;—about 52% to about 78% of humans of the group of humans survive for at least 12 months after day 1 of the 28-day study treatment cycle; and—about 26% to about 52% of humans of the group of humans survive for at least 24 months after day 1 of the 28-day study treatment cycle.

In some embodiments, the invention provides a method of treating cancer in a subject in need thereof, the method comprising administering to the subject a therapeutically effective amount of a DNA-hypomethylating agent on days 1-5 of a 28-day treatment cycle, wherein the therapeutically effective amount of the DNA-hypomethylating agent is about 90 mg per m² of body surface area of the subject, and wherein, in a controlled study:—each human of a group of humans has cancer;—90 mg per m² of body surface area of the DNA-hypomethylating agent is administered to each human of the group of humans on days 1-5 of a 28-day study treatment cycle;—about 45% to about 72% of humans of the group of humans survive for at least 12 months after day 1 of the 28-day study treatment cycle; and—about 18% to about 43% of humans of the group of humans survive for at least 24 months after day 1 of the 28-day study treatment cycle.

### BRIEF DESCRIPTION OF THE DRAWINGS

FIG. 1 shows overall survival (OS) of all patients by Compound I-I dose group.

FIG. 2 shows OS of treatment naive (TN) patients by Compound I-I dose group.

FIG. 3 shows OS of previously treated patients by Compound I-I dose group. R/R=relapsed/refractory.

FIG. 4 shows a survival analysis comparing treatment naïve and previously treated patients who received at least 4, or less than 4, treatment cycles.

FIG. 5 shows a survival analysis comparing treatment naïve and previously treated patients who received at least 6, or less than 6, treatment cycles.

FIG. 6 shows OS for patients receiving Compound I-I treatment vs. treatment choice (TC) of azacitidine, decitabine, or low dose Ara-C(LDAC).

FIG. 7 shows a landmark survival analysis of patients 4 months after the initiation of treatment with Compound I-I or TC.

FIG. 8 shows a Kaplan-Meier plot of overall survival in subject who received 4 or more cycles of treatment.

FIG. 9 shows a Kaplan-Meier plot of overall survival in subject who received 6 or more cycles of treatment.

FIG. 10 is a landmark analysis of patients who received fewer than or at least 4 cycles of treatment.

FIG. 11 is a landmark analysis of patients who received fewer than or at least 6 cycles of treatment.

FIG. 12 shows the overall survival by number of cycles in patients who were alive at 5 months with no objective response.

FIG. 13 provides a depiction of event-free survival (EFS) in the primary intention-to-treat (ITT) population.

FIG. 14 shows the EFS in the subgroup that received at least 4 cycles of treatment.

FIG. 15 demonstrates the EFS in the subgroup who received at least 6 cycles of treatment.

FIG. 16 demonstrates the EFS in responders who had at least composite complete response (CRc) or partial response (PR).

FIG. 17 depicts overall survival of patients who received fewer than 4 cycles of treatment or at least 4 cycles of treatment.

FIG. 18 depicts overall survival of patients with no CRc who received at least 4 cycles of treatment.

FIG. 19 depicts overall survival of patients with no CRc who received at least 6 cycles of treatment.

FIG. 20 provides the study design for the study described in EXAMPLE 9.

FIG. 21 depicts overall survival of patients with no CRc who received at least 6 cycles of treatment.

FIG. 22 shows the survival curves for patients with complete response (CR), CR with incomplete blood count recovery/CR with incomplete platelet recovery (CRi/CRp), and for patients who were non-responders.

FIG. 23 shows overall survival for CRc patients who went on to receive hematopoietic cell transplant (HCT) post CRc and CRc patients who did not receive HCT post treatment.

FIG. 24 is a Kaplan-Meier plot showing the overall survival of patients who were on a 5- or 10-day treatment regimen.

FIG. 25 shows the logistic regression for probability of complete response versus decitabine adjusted area under the curve (Compound I-1 arm).

FIG. 26 is a Kaplan-Meier plot for time to overall survival by the tertiles of decitabine adjusted area under the curve (Compound I-1 arm).

### DETAILED DESCRIPTION

Described herein is a method for the treatment of cancer with a DNA hypomethylating agent (DHA). A DHA of the disclosure can reverse aberrant DNA methylation patterns via the inhibition of DNA methyltransferase (DNMT). In some embodiments, a DHA of the disclosure is administered to a subject for at least 1, at least 2, at least 3, at least 4, at least 5, or at least 6 treatment cycles.

### DNA Methylation and Epigenetics.

Epigenetic modification of the genome, and in particular DNA methylation, plays a major role in the regulation of many normal cellular processes. DNA methylation is mediated by the enzyme DNA methyltransferase (DNMT). A DNMT catalyzes the addition of a methyl group to a cytosine nucleotide when the cytosine nucleotide is followed by a guanine nucleotide in a linear sequences of bases along the 5′ to 3′ direction. The cytosine residue followed by a guanine forms a CpG site. In humans, DNA methylation occurs at the 5 position of the pyrimidine ring of cytosine residues within CpG sites. CpG sites occur with high frequency in the promoter region of many mammalian genes. Genomic areas where CpG sites frequently occur are known as CpG islands.

Generally, promoter-associated CpG islands are unmethylated in non-malignant cells. DNA methylation of promoter-associated CpG islands results in silencing of the corresponding gene. Aberrant DNA hypermethylation is one of the major mechanisms leading to the inactivation of tumor suppressor genes. Hypermethylation of the promoter of, for example, tumor suppressor genes can affect hallmark processes associated with cancer such as genomic instability, increased cellular proliferation, decreased apoptosis, increased invasion and metastasis, and tumor immune invasion. Such cellular changes can promote oncogenesis, mediate tumor escape from host immune recognition, and result in a reduction in the clinical efficacy of cancer therapies.

Epigenetic-mediated silencing caused by abnormal DNA methylation can be reversed through the action of DNA hypomethylating agents (DHAs). For example, DHAs of the disclosure can reverse hypermethylation via the inhibition of DNMTs. Reversal of aberrant DNA hypermethylation can restore the expression of previously silenced tumor suppressor genes. Upregulation of previously silenced tumor suppressor genes can lead to decreased cell proliferation, increased apoptosis, and the sensitization or resensitization of tumor cells to anti-cancer therapies such as chemotherapeutics or immunotherapies.

### Assessment of Gene Mutational Status.

To assess the mutational status of a gene in a subject, a biological sample can be obtained from the subject and analyzed with various assays. A biological sample can be solid matter or can be a fluid. A biological fluid can include any fluid associated with living organisms. Non-limiting examples of a biological sample include cells, blood, components of blood such as serum, plasma, white blood cells, red blood cells, and platelets; tissue, cavity fluids, sputum, pus, microbiota, meconium, breast milk, saliva, urine, gastric and digestive fluid, tears, ocular fluids, sweat, mucus, earwax, oil, glandular secretions, spinal fluid, hair, fingernails, spinal fluid, cord blood, emphatic fluids, nasal excretions, and cell free samples such as DNA and RNA. A biological sample can be obtained from any anatomical location of a subject including, for example, heart, lung, kidney, breath, bone marrow, stool, semen, blood, blood vessels, lymphatic vessels, vaginal fluid, tumorous tissue, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, prostate, esophagus, thyroid, and/or other excretions or body tissues. In some embodiments, a biological sample is healthy tissue or healthy cells. In some embodiments, a biological sample is cancerous tissue or cancerous cells (e.g., leukemic cells).

Various methods and assays can be used to analyze a biological sample and assess the mutational status of a gene in a subject. Non-limiting examples of assays include DNA-sequencing, pyrosequencing, dye-terminator sequencing, massively parallel signature sequencing, sequencing by oligonucleotide ligation and detection (SOLiD), ion semiconductor sequencing, DNA nanoball sequencing, exome sequencing, RNA sequencing, bisulfite sequencing, fluorescent in situ sequencing, polymerase chain reaction, restriction fragment length polymorphism, microarrays, Southern blot, northern blot, western blot, and single nucleotide polymorphism arrays. In some embodiments, a DNA sequence is amplified via polymerase chain reaction (PCR) before the nucleotide sequence of the DNA sequence is determined.

In pyrosequencing, a single strand DNA template to be sequenced is hybridized to a sequencing primer and a DNA strand that is complementary to the DNA template is synthesized enzymatically. The DNA template strand hybridized to the primer is incubated with DNA polymerase, ATP sulfurylase, luciferase, apyrase, adenosine 5′ phosphosulfate (APS) and luciferin. To facilitate synthesis of the complementary strand one nucleotide at a time, one of four deoxynucleotide triphosphates (dNTPs) is added to the reaction mixture. When the correct dNTP is added, the incorporation of the dNTP into the growing complementary strand results in the release of light which can be detected. When an incorrect dNTP is added, the dNTP is degraded by the apyrase. Following incorporation of the correct dNTP, the process then proceeds with another nucleotide. By keeping track of which dNTP addition causes the release of light at each step, the sequence of the single strand DNA template can be determined.

Dye-terminator sequencing is based on the selective incorporation of chain terminating dideoxynucleotides by DNA polymerase during in vitro DNA replication. Copies of a DNA template are hybridized to a primer and incubated with dATP, dGTP, dCTP, dTTP, DNA polymerase, and the four dideoxynucleotides (ddATP, ddGTP, ddCTP, and ddTTP). The concentration of dideoxynucleotides in the reaction mixture is about 100 times less than the concentration of deoxynucleotides. Each dideoxynucleotide is labelled with a different fluorescent dye. DNA strands that are complementary to the template strand are synthesized, with synthesis of each strand stopping when a dideoxynucleotide is incorporated into the growing strand. Resulting DNA fragments are then heat denatured and separated by size using gel electrophoresis. The nucleobase present at the end of each separated DNA fragment can be determined based on the fluorescent dye associated with each dideoxynucleotide.

Massively parallel signature sequencing (MPSS) can identify and quantify mRNA transcripts. mRNA is converted to complementary DNA (cDNA), and the cDNA is fused to a small oligonucleotide, PCR amplified, and coupled to microbeads. The sequences of cDNA samples are then determined via the hybridization of fluorescently labeled encoders which hybridize with four nucleotide bases. After a round of imaging to identify the encoder on each microbead, the next step is to cleave and remove the four hybridized bases to reveal the next four bases for a new round of encoder hybridization and image acquisition. Each encoder corresponds to a distinct four-nucleotide sequence allowing for the identification of the microbead-bound cDNA sequences.

In SOLiD sequencing, a single strand of unknown DNA sequence is flanked on at least one end by a known sequence on the surface of a magnetic bead. The DNA strand is then exposed to a primer strand that binds the known sequence. The DNA strand-primer complex is then exposed to a mixed pool of fluorescently labeled probes that are eight bases long. The first two bases of the probe correspond to each possible dinucleotide permutation, the next three bases are universal bases that bind to any of the four dinucleotides, and the final three bases are universal bases labeled with fluorescent dye. Probe molecules hybridize to the target DNA sequence, next to the primer sequence. DNA ligase preferentially joins the probe molecule to the primer when the first two bases of the probe molecule are complementary to the corresponding bases of the unknown DNA sequence. Following a round of ligation and fluorescent imaging, the dye-containing bases of the probe molecule are cleaved to complete a cycle. Multiple cycles are performed until the desired read length is achieved. Following a series of cycles, extension product (formed by the ligated probes) is removed and the entire process is repeated four times, each time with the primer offset by one base so that a fluorescent measurement can be recorded for every base. The sequence of the unknown strand can then be inferred via analysis of fluorescent data.

Ion semiconductor sequencing detects the addition of a nucleic acid residue as an electrical signal associated with a hydrogen ion liberated during synthesis. A reaction well containing a template is flooded with the four types of nucleotide building blocks, one at a time. The timing of the electrical signal identifies which building block was added and identifies the corresponding residue in the template.

DNA nanoball sequencing involves isolating DNA, shearing the DNA into 100-350 base pair fragments, ligating adapter sequences to the fragments, and circularizing the fragments. Circular fragments are then copied via rolling circle replication to produce single stranded copies of each fragment. Single stranded copies concatenate head to tail in a long strand and are compacted into a nanoball. Nanoballs are then adsorbed onto a sequencing flow cell. Fluorescently labeled nucleotides are then incorporated into a growing strand complementary to the unknown DNA sequence and the fluorescence from DNA nanoballs is recorded. The color of fluorescence corresponds to a nucleotide base at the interrogated position.

### DNA Hypomethylating Agents.

The compounds disclosed herein can be effective as DHAs. The compounds of the disclosure can promote DNA hypomethylation by, for example, inhibiting DNMTs. Such compounds can inhibit DNMTs by, for example, inducing the incorporation of metabolites into actively replicating DNA strands.

In some embodiments, a compound of the disclosure is a compound of Formula I or a pharmaceutically acceptable salt thereof:

(5-azacytosine group)-L-(guanine group) (I),

wherein L is a phosphorous-containing linker wherein the number of phosphorous atoms in L is 1.

In some embodiments, L is a group suitable for linking the 5-azacytosine group with the guanine group. In some embodiments, L comprises a carbohydrate. In some embodiments, L comprises more than one carbohydrate. In some embodiments, L comprises two carbohydrates. When L comprises more than one carbohydrate, the carbohydrates can be the same or different. A carbohydrate can be a monosaccharide in the closed ring form, such as a pyranose or furanose form. A carbohydrate can be substituted at any position or deoxygenated at any position that would be oxygenated in a naturally-occurring form of the carbohydrate. In some embodiments, the carbohydrate is ribose. In some embodiments, the carbohydrate is 2-deoxyribose. The ribose or 2-deoxyribose can be substituted at any position.

The phosphate atom of L can be present in any naturally-occurring or synthetic functional group containing a phosphorus atom. Non-limiting examples of such functional groups include phosphodiesters, phosphorothioate diesters, boranophosphate diesters, and methylphosphonate diesters.

In some embodiments, L comprises Formula II. In some embodiments, L is Formula II:

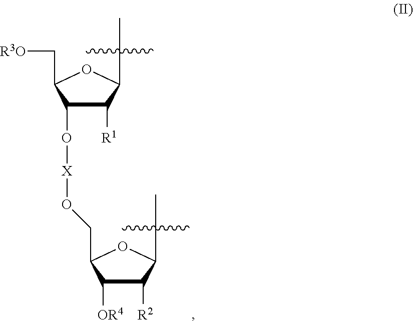

wherein, R¹ and R² are independently H, OH, an alkoxy group, an alkoxyalkoxy group, an acyloxy group, a carbonate group, a carbamate group, or a halogen; R³ is H, or R³ together with the oxygen atom to which R³ is bound forms an ether, an ester, a carbonate, or a carbamate; R⁴ is H, or R⁴ together with the oxygen atom to which R⁴ is bound forms an ether, an ester, a carbonate, or a carbamate; and X together with the oxygen atoms to which X is bound forms a phosphodiester, a phosphorothioate diester, a boranophosphate diester, or a methylphosphonate diester.

In some embodiments, the 5-azacytosine group can be linked to either end of L, and the guanine group can be linked to the other end of L and the compound contains one 5-azacytosine group and one guanine group. Constitutional isomers can thus be prepared by exchanging the connectivity of the 5-azacytosine group and the guanine group.

R¹ and R² can be the same or different. In some embodiments, R¹ and R² are independently H, OH, OMe, OEt, OPh, OCH₂CH₂OMe, OCH₂CH₂OEt, OCH₂CH₂OBn, OBn, OAc, OBz, OCOOMe, OCOOEt, OCOOBn, OCONH₂, OCONMe₂, OCONEt₂, OCONBn₂, OCONHMe, OCONHEt, OCONHBn, F, Cl, Br, or I. In some embodiments, R¹ and R² are independently H, OH, OMe, OEt, OCH₂CH₂OMe, OBn, or F. In some embodiments, R¹ and R² are independently H or OH. In some embodiments, R¹ and R² are H. In some embodiments, R¹ and R² are OH.

R³ and R⁴ can be the same or different.

In some embodiments, R³ is H, or R³ together with the oxygen atom to which R³ is bound forms OH, OMe, OEt, OPh, OCH₂CH₂OMe, OCH₂CH₂OEt, OCH₂CH₂OBn, OBn, OAc, OBz, OCOOMe, OCOOEt, OCOOBn, OCONH₂, OCONMe₂, OCONEt₂, OCONBn₂, OCONHMe, OCONHEt, or OCONHBn. In some embodiments, R³ is H, or R³ together with the oxygen atom to which R³ is bound forms OH, OMe, OEt, OCH₂CH₂OMe, or OBn. In some embodiments, R³ is H.

In some embodiments, R⁴ is H, or R⁴ together with the oxygen atom to which R⁴ is bound forms OH, OMe, OEt, OPh, OCH₂CH₂OMe, OCH₂CH₂OEt, OCH₂CH₂OBn, OBn, OAc, OBz, OCOOMe, OCOOEt, OCOOBn, OCONH₂, OCONMe₂, OCONEt₂, OCONBn₂, OCONHMe, OCONHEt, or OCONHBn. In some embodiments, R⁴ is H, or R⁴ together with the oxygen atom to which R⁴ is bound forms OH, OMe, OEt, OCH₂CH₂OMe, or OBn. In some embodiments, R⁴ is H.

In some embodiments, X is P(O)OH, P(O)SH, P(→O)BH₃⁻, or P(O)Me. In some embodiments, X is P(O)OH. In some embodiments, X together with the oxygen atoms to which X is bound forms a phosphodiester.

Non-limiting examples of alkyl include straight, branched, and cyclic alkyl groups. Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight or branched alkyl groups.

A halo-alkyl group can be any alkyl group substituted with any number of halogen atoms, for example, fluorine, chlorine, bromine, and iodine atoms.

An alkoxy group can be, for example, an oxygen atom substituted with any alkyl group. An ether or an ether group comprises an alkoxy group. Non-limiting examples of alkoxy groups include methoxy, ethoxy, propoxy, isopropoxy, and isobutoxy.

An alkoxyalkoxy group can be, for example, an alkoxy group substituted at any position with any alkoxy group. Non-limiting examples of alkoxyalkoxy groups include methoxyethoxy, ethyoxyethoxy, ethoxyethoxyethoxy, groups derived from any order of glyme, and groups derived from polyethylene glycol.

An aryl group can be heterocyclic or non-heterocyclic. An aryl group can be monocyclic or polycyclic. An aryl group can be substituted with any number of hydrocarbyl groups, alkyl groups, and halogen atoms. Non-limiting examples of aryl groups include phenyl, toluyl, naphthyl, pyrrolyl, pyridyl, imidazolyl, thiophenyl, and furyl.

An aryloxy group can be, for example, an oxygen atom substituted with any aryl group, such as phenoxy.

An aralkyl group can be, for example, any alkyl group substituted with any aryl group, such as benzyl.

An arylalkoxy group can be, for example, an oxygen atom substituted with any aralkyl group, such as benzyloxy.

A heterocycle can be any ring containing a ring atom that is not carbon. A heterocycle can be substituted with any number of alkyl groups and halogen atoms. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

An acyl group can be, for example, a carbonyl group substituted with hydrocarbyl, alkyl, hydrocarbyloxy, alkoxy, aryl, aryloxy, aralkyl, arylalkoxy, or a heterocycle. Non-limiting examples of acyl include acetyl, benzoyl, benzyloxycarbonyl, phenoxycarbonyl, methoxycarbonyl, and ethoxycarbonyl.

An acyloxy group can be an oxygen atom substituted with an acyl group. An ester or an ester group comprises an acyloxy group.

A carbonate group can be an oxygen atom substituted with hydrocarbyloxycarbonyl, alkoxycarbonyl, aryloxycarbonyl, or aryl alkoxycarbonyl.

A carbamate group can be an oxygen atom substituted with a carbamoyl group, wherein the nitrogen atom of the carbamoyl group is unsubstituted, monosubstituted, or disubstituted with one or more of hydrocarbyl, alkyl, aryl, heterocyclyl, or aralkyl. When the nitrogen atom is disubstituted, the two substituents together with the nitrogen atom can form a heterocycle.

Any functional group of a compound described herein can be optionally capped with a capping group. For examples of capping groups, see GREENE'S PROTECTIVE GROUPS IN ORGANIC SYNTHESIS, 4th Ed. (Wiley 2006) (1980) and PROTECTING GROUPS, 3d Ed. (Thieme 2005) (1994), each of which is incorporated by reference in its entirety.

Non-limiting examples of suitable capping groups for a hydroxyl group include alkyl, haloalkyl, aryl, aralkyl, carbonate, carbamate, and acyl groups.

Non-limiting examples of suitable capping groups for nitrogen-functionalities include alkyl, aryl, aralkyl, an acyl group, an alkoxycarbonyl group, an aryloxycarbonyl group, and an aminocarbonyl group. A capping group together with the nitrogen atom to which the capping group is bound can form, for example, an amide, a carbamate, a urethane, a heterocycle, or an amine. Two capping groups bound to the same nitrogen atom can form together with the nitrogen atom a heterocycle.

The disclosure provides pharmaceutically-acceptable salts of any compound described herein. Pharmaceutically-acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some embodiments, a pharmaceutically-acceptable salt is a metal salt. In some embodiments, a pharmaceutically-acceptable salt is an ammonium salt.

Acid addition salts can arise from the addition of an acid to a compound described herein. In some embodiments, the acid is organic. In some embodiments, the acid is inorganic. Non-limiting examples of suitable acids include hydrochloric acid, hydrobromic acid, hydroiodic acid, nitric acid, nitrous acid, sulfuric acid, sulfurous acid, a phosphoric acid, nicotinic acid, isonicotinic acid, lactic acid, salicylic acid, 4-aminosalicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, citric acid, oxalic acid, maleic acid, hydroxymaleic acid, methylmaleic acid, glycolic acid, malic acid, cinnamic acid, mandelic acid, 2-phenoxybenzoic acid, 2-acetoxybenzoic acid, embonic acid, phenylacetic acid, N-cyclohexylsulfamic acid, methanesulfonic acid, ethanesulfonic acid, benzenesulfonic acid, p-toluenesulfonic acid, 2-hydroxyethanesulfonic acid, ethane-1,2-disulfonic acid, 4-methylbenzenesulfonic acid, naphthalene-2-sulfonic acid, naphthalene-1,5-disulfonic acid, 2-phosphoglyceric acid, 3-phosphoglyceric acid, glucose-6-phosphoric acid, and an amino acid.

Non-limiting examples of suitable acid addition salts include a hydrochloride salt, a hydrobromide salt, a hydroiodide salt, a nitrate salt, a nitrite salt, a sulfate salt, a sulfite salt, a phosphate salt, a hydrogen phosphate salt, a dihydrogen phosphate salt, a carbonate salt, a bicarbonate salt, a nicotinate salt, an isonicotinate salt, a lactate salt, a salicylate salt, a 4-aminosalicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a citrate salt, an oxalate salt, a maleate salt, a hydroxymaleate salt, a methylmaleate salt, a glycolate salt, a malate salt, a cinnamate salt, a mandelate salt, a 2-phenoxybenzoate salt, a 2-acetoxybenzoate salt, an embonate salt, a phenylacetate salt, an N-cyclohexylsulfamate salt, a methanesulfonate salt, an ethanesulfonate salt, a benzenesulfonate salt, a p-toluenesulfonate salt, a 2-hydroxyethanesulfonate salt, an ethane-1,2-disulfonate salt, a 4-methylbenzenesulfonate salt, a naphthalene-2-sulfonate salt, a naphthalene-1,5-disulfonate salt, a 2-phosphoglycerate salt, a 3-phosphoglycerate salt, a glucose-6-phosphate salt, and an amino acid salt.

Metal salts can arise from the addition of an inorganic base to a compound described herein. The inorganic base consists of a metal cation paired with a basic counterion, such as, for example, hydroxide, carbonate, bicarbonate, or phosphate. The metal can be an alkali metal, alkaline earth metal, transition metal, or main group metal. Non-limiting examples of suitable metals include lithium, sodium, potassium, caesium, cerium, magnesium, manganese, iron, calcium, strontium, cobalt, titanium, aluminium, copper, cadmium, and zinc.

Non-limiting examples of suitable metal salts include a lithium salt, a sodium salt, a potassium salt, a caesium salt, a cerium salt, a magnesium salt, a manganese salt, an iron salt, a calcium salt, a strontium salt, a cobalt salt, a titanium salt, a aluminium salt, a copper salt, a cadmium salt, and a zinc salt.

Ammonium salts can arise from the addition of ammonia or an organic amine to a compound described herein. Non-limiting examples of suitable organic amines include triethyl amine, diisopropyl amine, ethanol amine, diethanol amine, triethanol amine, morpholine, N-methylmorpholine, piperidine, N-methylpiperidine, N-ethylpiperidine, dibenzyl amine, piperazine, pyridine, pyrrazole, pipyrrazole, imidazole, pyrazine, pipyrazine, ethylenediamine, N,N′-dibenzylethylene diamine, procaine, chloroprocaine, choline, dicyclohexyl amine, and N-methylglucamine.

Non-limiting examples of suitable ammonium salts include is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzyl amine salt, a piperazine salt, a pyridine salt, a pyrrazole salt, a pipyrrazole salt, an imidazole salt, a pyrazine salt, a pipyrazine salt, an ethylene diamine salt, an N,N′-dibenzylethylene diamine salt, a procaine salt, a chloroprocaine salt, a choline salt, a dicyclohexyl amine salt, and a N-methylglucamine salt.

Non-limiting examples of compounds of Formula I include Compounds I-1-I-44 as shown below:

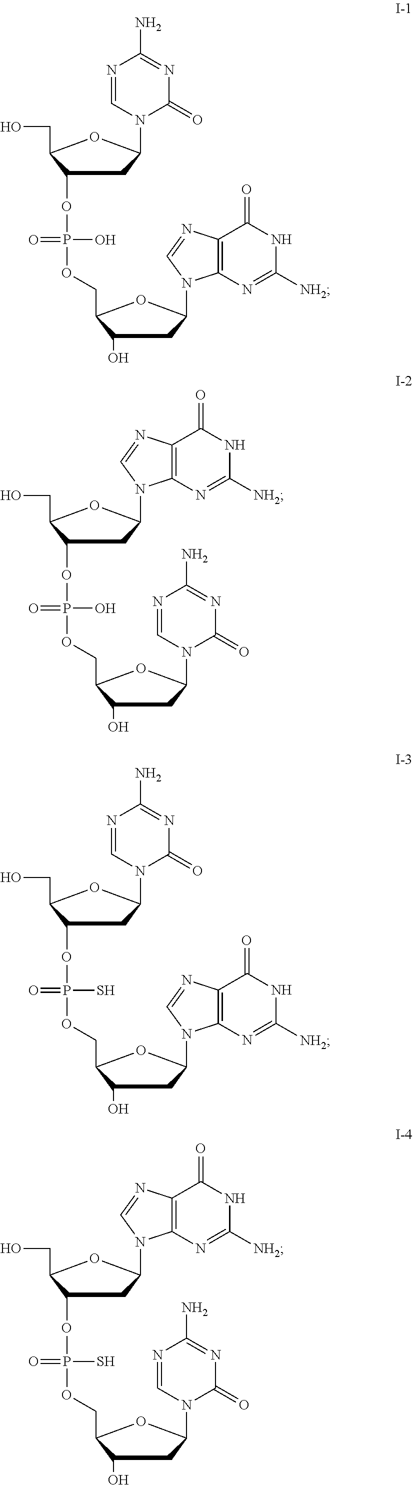 [SMILES: Nc1ncn([C@H]2C[C@@H](OP(=O)(O)OC[C@H]3O[C@@H](n4cnc5c(=O)[nH]c(N)nc54)C[C@H]3O)[C@@H](CO)O2)c(=O)n1 ; Nc1ncn([C@H]2C[C@@H](O)[C@@H](COP(=O)(O)O[C@@H]3C[C@H](n4cnc5c(=O)[nH]c(N)nc54)O[C@@H]3CO)O2)c(=O)n1 ; Nc1ncn([C@H]2C[C@@H](OP(=O)(S)OC[C@H]3O[C@@H](n4cnc5c(=O)[nH]c(N)nc54)C[C@H]3O)[C@@H](CO)O2)c(=O)n1 ; Nc1ncn([C@H]2C[C@@H](O)[C@@H](COP(=O)(S)O[C@@H]3C[C@H](n4cnc5c(=O)[nH]c(N)nc54)O[C@@H]3CO)O2)c(=O)n1]

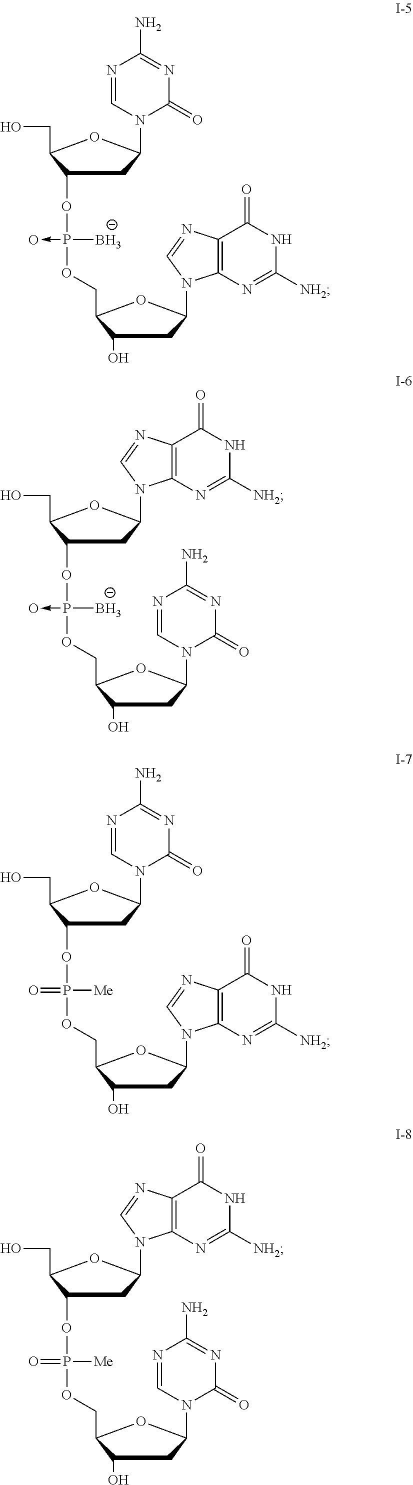

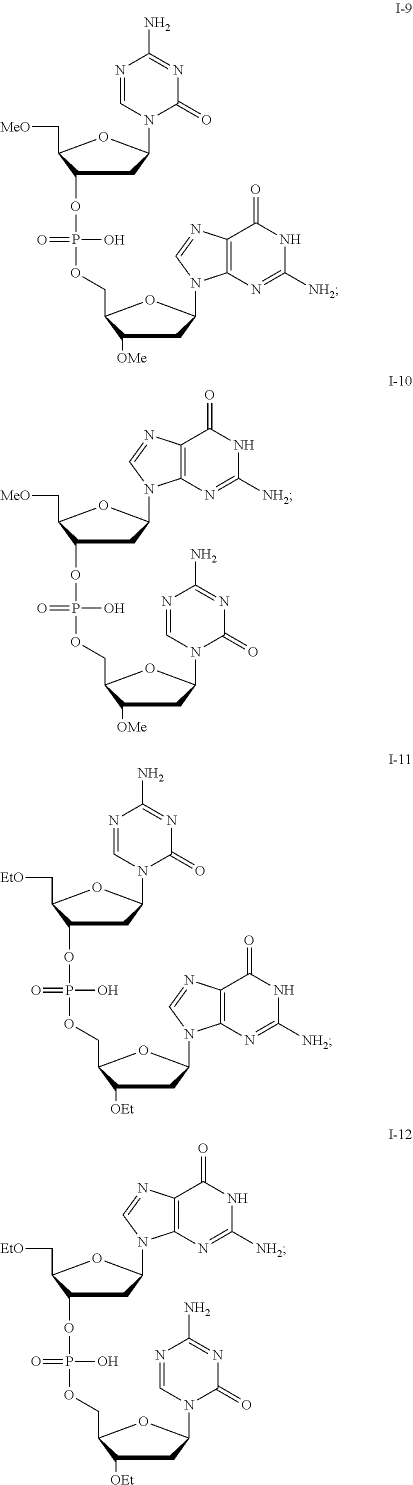 [SMILES: COC[C@H]1O[C@@H](n2cnc(N)nc2=O)C[C@H]1OP(=O)(O)OC[C@H]1O[C@@H](n2cnc3c(=O)[nH]c(N)nc32)C[C@H]1OC ; COC[C@H]1O[C@@H](n2cnc3c(=O)[nH]c(N)nc32)C[C@H]1OP(=O)(O)OC[C@H]1O[C@@H](n2cnc(N)nc2=O)C[C@H]1OC ; CCOC[C@H]1O[C@@H](n2cnc(N)nc2=O)C[C@H]1OP(=O)(O)OC[C@H]1O[C@@H](n2cnc3c(=O)[nH]c(N)nc32)C[C@H]1OCC ; CCOC[C@H]1O[C@@H](n2cnc3c(=O)[nH]c(N)nc32)C[C@H]1OP(=O)(O)OC[C@H]1O[C@@H](n2cnc(N)nc2=O)C[C@H]1OCC]

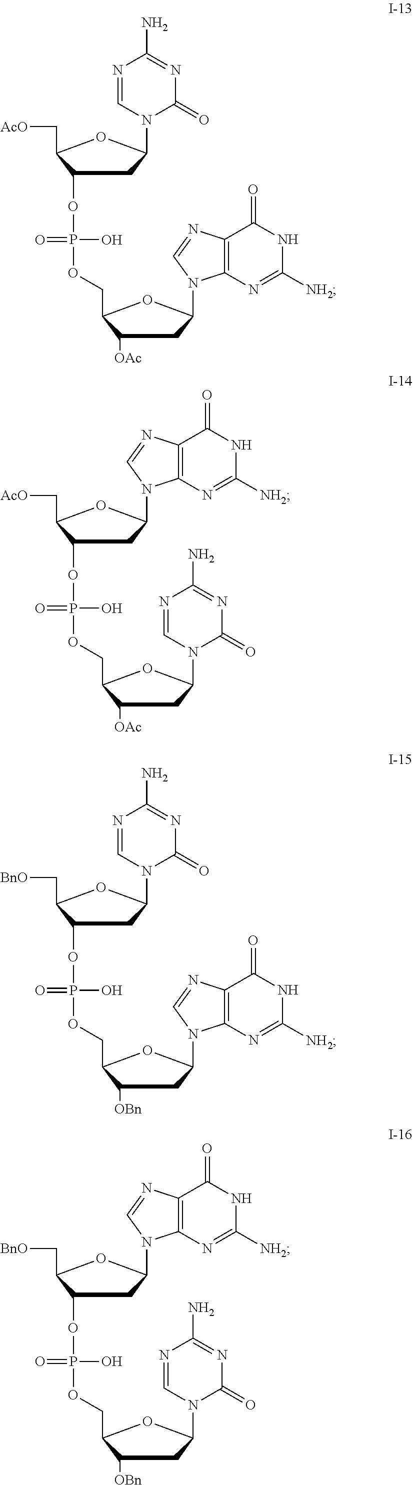 [SMILES: CC(=O)OC[C@H]1O[C@@H](n2cnc(N)nc2=O)C[C@H]1OP(=O)(O)OC[C@H]1O[C@@H](n2cnc3c(=O)[nH]c(N)nc32)C[C@H]1OC(C)=O ; CC(=O)OC[C@H]1O[C@@H](n2cnc3c(=O)[nH]c(N)nc32)C[C@H]1OP(=O)(O)OC[C@H]1O[C@@H](n2cnc(N)nc2=O)C[C@H]1OC(C)=O ; Nc1ncn([C@H]2C[C@@H](OP(=O)(O)OC[C@H]3O[C@@H](n4cnc5c(=O)[nH]c(N)nc54)C[C@H]3OCc3ccccc3)[C@@H](COCc3ccccc3)O2)c(=O)n1 ; Nc1ncn([C@H]2C[C@@H](OCc3ccccc3)[C@@H](COP(=O)(O)O[C@@H]3C[C@H](n4cnc5c(=O)[nH]c(N)nc54)O[C@@H]3COCc3ccccc3)O2)c(=O)n1]

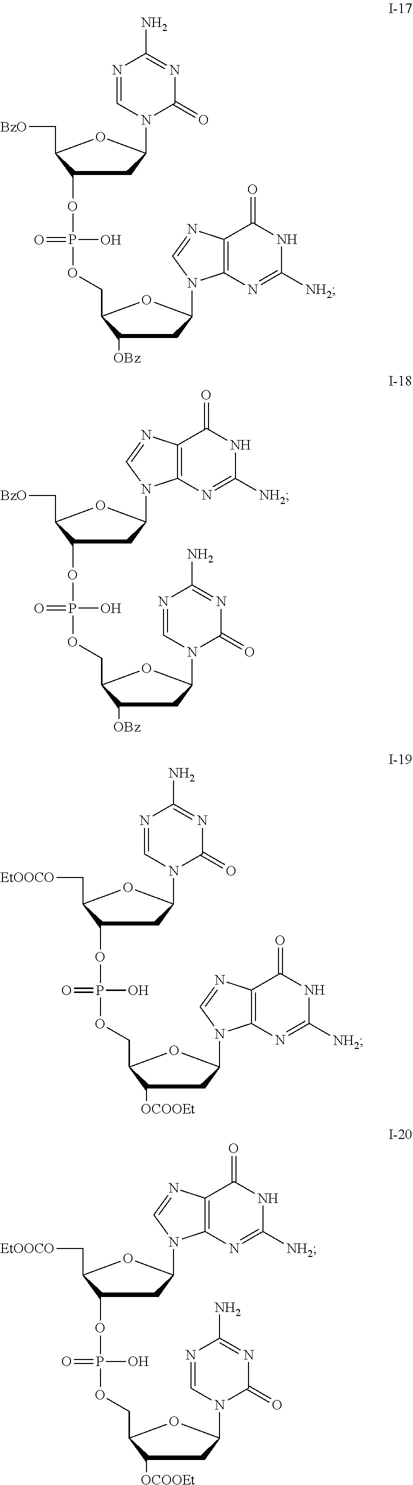

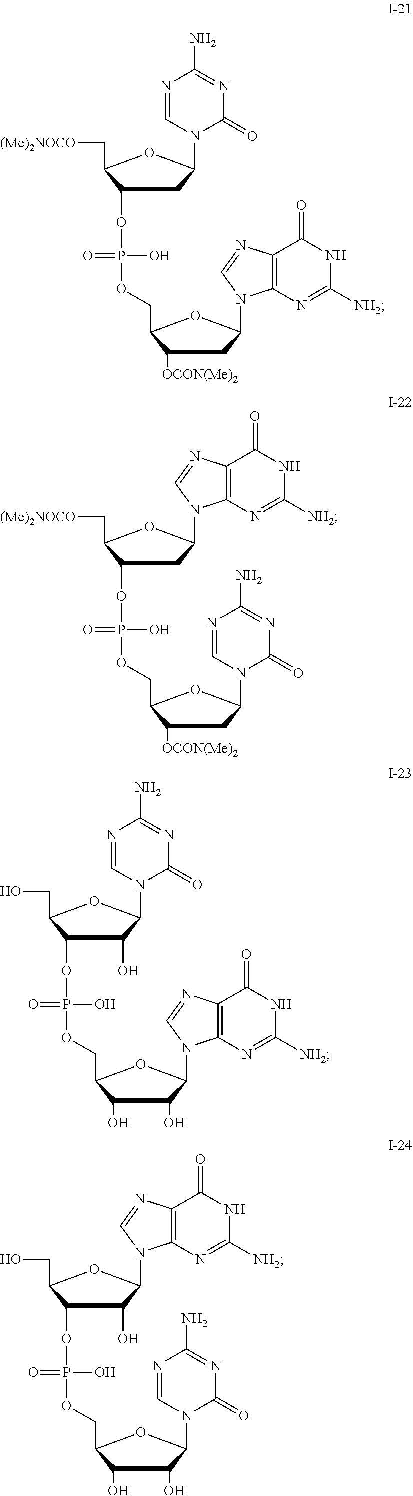

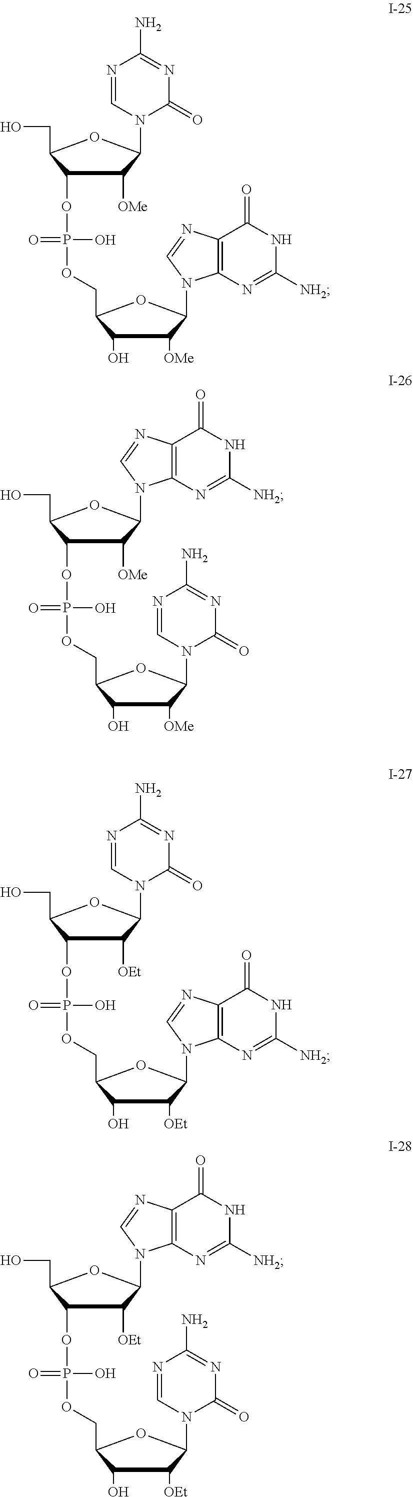

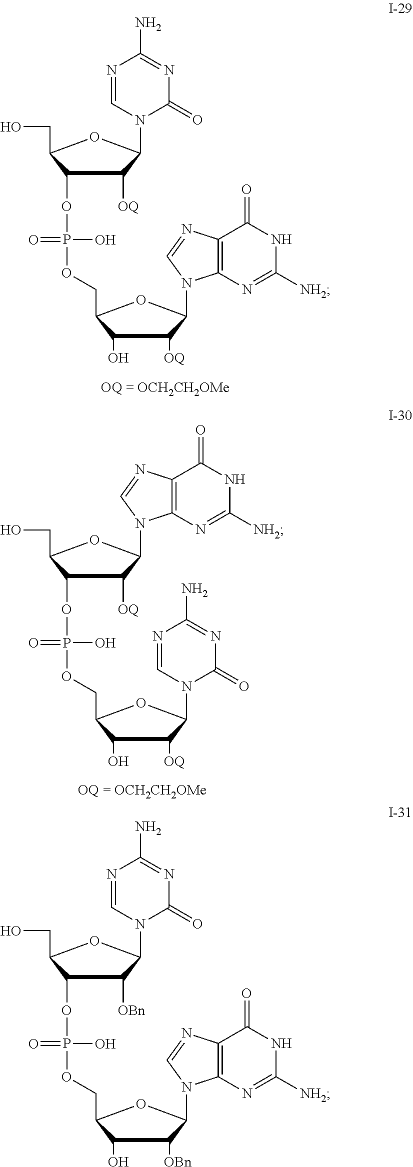

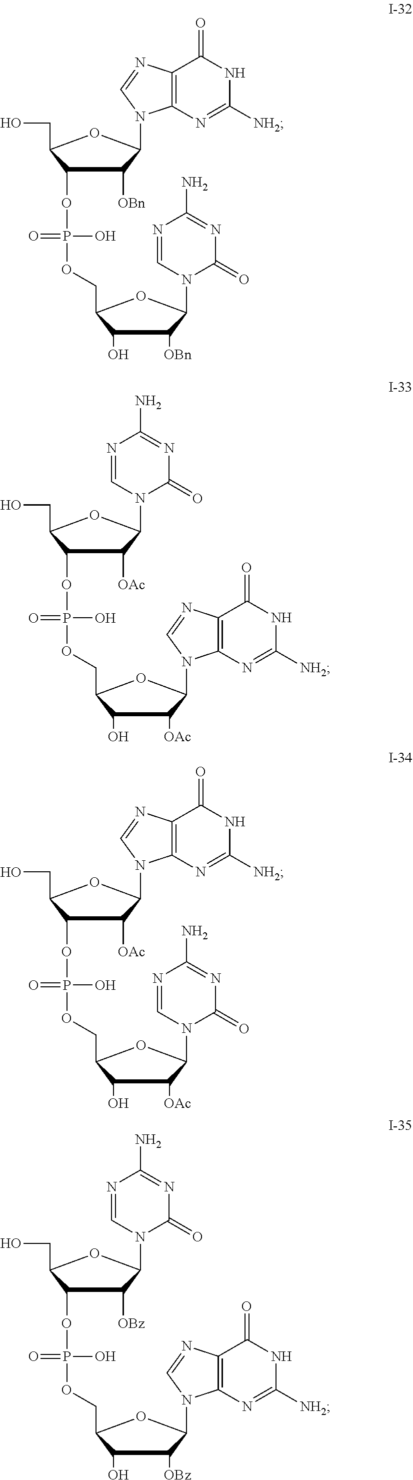

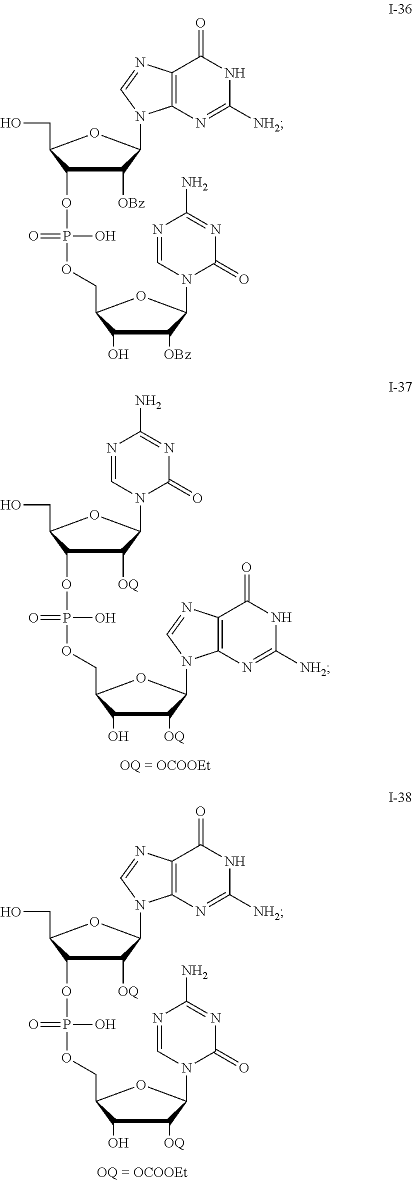

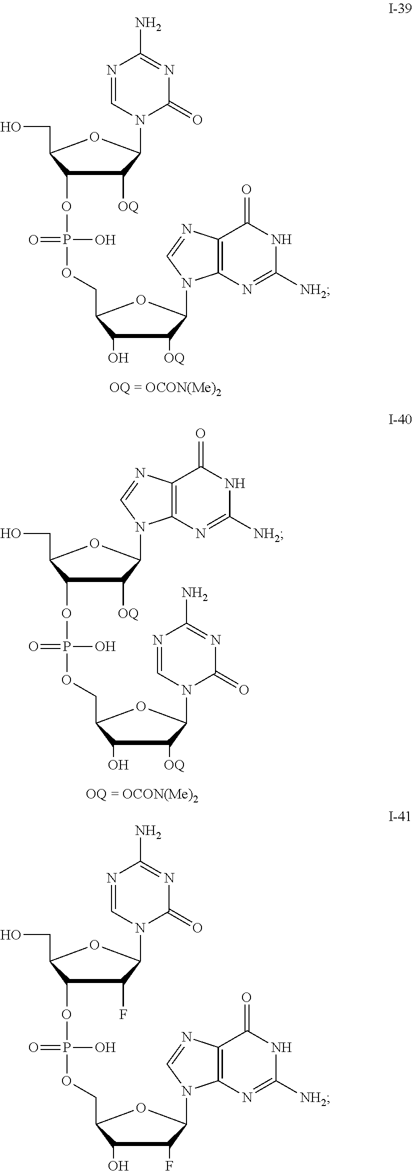

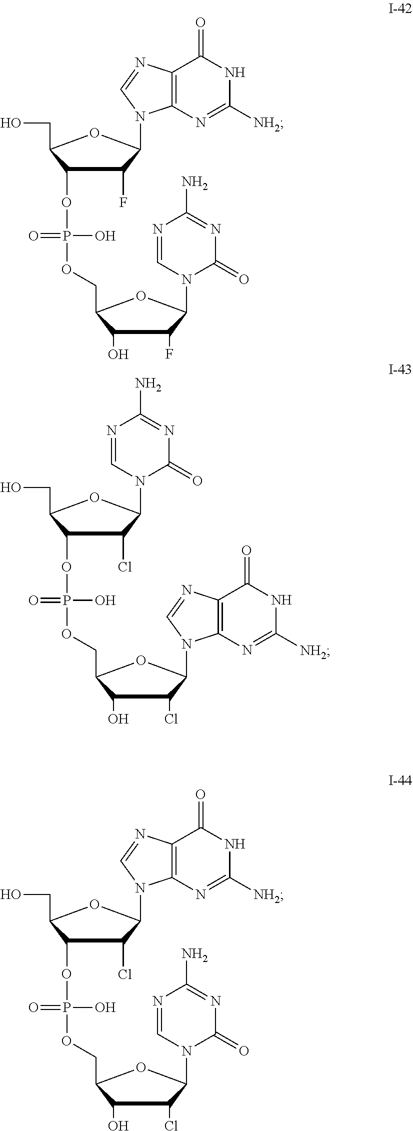

and pharmaceutically-acceptable salts of any of the foregoing. In some embodiments, a salt is a sodium salt of any of the foregoing.

The compounds described herein can be synthesized by, for example, solution phase or solid phase synthesis.

### Pharmaceutical Compositions.

Compounds disclosed herein can be provided as part of a pharmaceutical composition. Non-limiting examples of pharmaceutical compositions include any composition suitable for administration to a subject, for example, in a form, concentration and/or level of purity suitable for administration to a human or animal subject. In some embodiments, pharmaceutical compositions are sterile and/or non-pyrogenic. A non-pyrogenic pharmaceutical composition does not elicit undesirable inflammatory responses when administered to a subject.

In some instances, a pharmaceutical composition of the disclosure is a formulation comprising a DHA. Suitable formulations can be solutions or suspensions of a compound in a solvent or a mixture of solvents. Non-limiting examples of suitable solvents include propylene glycol, glycerin, ethanol, and any combination of the foregoing. The formulations can be prepared as non-aqueous formulations. The formulations can be anhydrous or substantially anhydrous.

A mixture of solvents can contain a percentage of propylene glycol on either a mass or a volume basis. In some embodiments, the percentage of propylene glycol can be at least 10%, at least 20%, at least 30%, at least 40%, at least 50%, at least about 10%, at least about 20%, at least about 30%, at least about 40%, or at least about 50%. In some embodiments, the percentage of propylene glycol can be at most 90%, at most 80%, at most 70%, at most 60%, at most about 90%, at most about 80%, at most about 70%, or at most about 60%. In some embodiments, the percentage of propylene glycol can be 30% to 90%, 45% to 85%, 55% to 75%, 60% to 70%, about 30% to about 90%, about 45% to about 85%, about 55% to about 75%, or about 60% to about 70%. In some embodiments, the percentage of propylene glycol can be 30%, 35%, 40%, 45%, 50%, 55%, 60%, 65%, 70%, 75%, 80%, 85%, 90%, about 30%, about 35%, about 40%, about 45%, about 50%, about 55%, about 60%, about 65%, about 70%, about 75%, about 80%, about 85%, or about 90%.

A mixture of solvents can contain a percentage of glycerin on either a mass or a volume basis. In some embodiments, the percentage of glycerin can be at least 5%, at least 10%, at least 15%, at least 25%, at least 30%, at least about 5%, at least about 10%, at least about 15%, at least about 25%, or at least about 30%. In some embodiments, the percentage of glycerin can be at most 70%, at most 60%, at most 50%, at most 40%, at most 30%, at most about 70%, at most about 60%, at most about 50%, at most about 40%, or at most about 30%. In some embodiments, the percentage of glycerin can be 0% to 50%, 5% to 45%, 15% to 35%, 20% to 30%, 0% to about 50%, about 5% to about 45%, about 15% to about 35%, or about 20% to about 30%. In some embodiments, the percentage of glycerin can be 0%, 5%, 10%, 15%, 20%, 25%, 30%, 35%, 40%, 45%, 50%, about 5%, about 10%, about 15%, about 20%, about 25%, about 30%, about 35%, about 40%, about 45%, or about 50%.

A mixture of solvents can contain a percentage of ethanol on either a mass or a volume basis. In some embodiments, the percentage of ethanol can be at least 1%, at least 3%, at least 5%, at least 10%, at least 15%, at least about 1%, at least about 3%, at least about 5%, at least about 10%, or at least about 15%. In some embodiments, the percentage of ethanol can be at most 30%, at most 25%, at most 20%, at most 15%, at most 10%, at most about 30%, at most about 25%, at most about 20%, at most about 15%, or at most about 10%. In some embodiments, the percentage of ethanol can be 0% to 30%, 0% to 25%, 0% to 20%, 5% to 15%, 0% to about 30%, 0% to about 25%, 0% to about 20%, or about 5% to about 15%. In some embodiments, the percentage of ethanol can be 0%, 1%, 2%, 3%, 4%, 5%, 6%, 7%, 8%, 9%, 10%, 11%, 12%, 13%, 14%, 15%, about 1%, about 2%, about 3%, about 4%, about 5%, about 6%, about 7%, about 8%, about 9%, about 10%, about 11%, about 12%, about 13%, about 14%, or about 15%.

In some embodiments, a solvent or a mixture of solvents comprises 45% to 85% propylene glycol, 5% to 45% glycerin, and 0% to 30% ethanol. In some embodiments, a solvent or a mixture of solvents comprises about 45% to about 85% propylene glycol, about 5% to about 45% glycerin, and 0% to about 30% ethanol. In some embodiments, a solvent or a mixture of solvents consists essentially of 45% to 85% propylene glycol, 5% to 45% glycerin, and 0% to 30% ethanol. In some embodiments, a solvent or a mixture of solvents consists essentially of about 45% to about 85% propylene glycol, about 5% to about 45% glycerin, and 0% to about 30% ethanol. In some embodiments, a solvent or a mixture of solvents is 45% to 85% propylene glycol, 5% to 45% glycerin, and 0% to 30% ethanol. In some embodiments, a solvent or a mixture of solvents is about 45% to about 85% propylene glycol, about 5% to about 45% glycerin, and 0% to about 30% ethanol.

In some embodiments, a solvent or a mixture of solvents comprises 55% to 75% propylene glycol, 15% to 35% glycerin, and 0% to 20% ethanol. In some embodiments, a solvent or a mixture of solvents comprises about 55% to about 75% propylene glycol, about 15% to about 35% glycerin, and 0% to about 20% ethanol. In some embodiments, a solvent or a mixture of solvents consists essentially of 55% to 75% propylene glycol, 15% to 35% glycerin, and 0% to 20% ethanol. In some embodiments, a solvent or a mixture of solvents consists essentially of about 55% to about 75% propylene glycol, about 15% to about 35% glycerin, and 0% to about 20% ethanol. In some embodiments, a solvent or a mixture of solvents is 55% to 75% propylene glycol, 15% to 35% glycerin, and 0% to 20% ethanol. In some embodiments, a solvent or a mixture of solvents is about 55% to about 75% propylene glycol, about 15% to about 35% glycerin, and 0% to about 20% ethanol.

In some embodiments, a solvent or a mixture of solvents comprises 60% to 70% propylene glycol; 20% to 30% glycerin; and 5% to 15% ethanol. In some embodiments, a solvent or a mixture of solvents comprises about 60% to about 70% propylene glycol; about 20% to about 30% glycerin; and about 5% to about 15% ethanol. In some embodiments, a solvent or a mixture of solvents consists essentially of 60% to 70% propylene glycol; 20% to 30% glycerin; and 5% to 15% ethanol. In some embodiments, a solvent or a mixture of solvents consists essentially of about 60% to about 70% propylene glycol; about 20% to about 30% glycerin; and about 5% to about 15% ethanol. In some embodiments, a solvent or a mixture of solvents is 60% to 70% propylene glycol; 20% to 30% glycerin; and 5% to 15% ethanol. In some embodiments, a solvent or a mixture of solvents is about 60% to about 70% propylene glycol; about 20% to about 30% glycerin; and about 5% to about 15% ethanol.

In some embodiments, a solvent or a mixture of solvents comprises 65% propylene glycol; 25% glycerin; and 10% ethanol. In some embodiments, a solvent or a mixture of solvents comprises about 65% propylene glycol; about 25% glycerin; and about 10% ethanol. In some embodiments, a solvent or a mixture of solvents consists essentially of 65% propylene glycol; 25% glycerin; and 10% ethanol. In some embodiments, a solvent or a mixture of solvents consists essentially of about 65% propylene glycol; about 25% glycerin; and about 10% ethanol. In some embodiments, a solvent or a mixture of solvents is 65% propylene glycol; 25% glycerin; and 10% ethanol. In some embodiments, a solvent or a mixture of solvents is about 65% propylene glycol; about 25% glycerin; and about 10% ethanol.

Formulations can be prepared utilizing dimethyl sulfoxide (DMSO) as a solvent. In some cases, the use of substantially anhydrous DMSO increases stability. Any source of DMSO can be used. In some embodiments, the DMSO source is suitable for healthcare and drug delivery applications, for example conforming to USP or Ph. Eur monographs, and can be manufactured under cGMP and API guidelines. Grades such as anhydrous or Pharma Solvent can be used according to the disclosure. In some embodiments, the DMSO can have impurities in very low levels, for example <0.2% water by KF, <0.01% non-volatile residue and <0.1% of related compounds. In further embodiments, the DMSO can include isosteres thereof, including DMSO isosteres in which one or more atom(s) is(are) replaced by a cognate isotope, for example hydrogen by deuterium.

Formulations of the disclosure can be prepared, stored, transported, and handled in anhydrous or substantially-anhydrous form. A solvent can be dried prior to preparing a formulation, and a compound can be dried, for example, by lyophilization. A drying agent, or desiccant, can be used during preparation, storage, transportation, or handling to regulate water content. Non-limiting examples of drying agents include silica gel, calcium sulfate, calcium chloride, calcium phosphate, sodium chloride, sodium bicarbonate, sodium sulfate, sodium phosphate, montmorillonite, molecular sieves (beads or powdered), alumina, titania, zirconia, and sodium pyrophosphate. A drying agent can contact a formulation directly, be inserted into the formulation in the form of a packet with a permeable membrane or be stored with the formulation in a sealed environment, such as a desiccator, such that the drying agent and the formulation are simultaneously exposed to the same controlled atmosphere. A drying agent can be removed from a formulation, for example, by filtration or cannulation. Additionally, a formulation can be stored in a sealed container within a controlled atmosphere consisting essentially of, or enriched in, nitrogen or argon.

Anhydrous or substantially-anhydrous conditions benefit the shelf-life of a formulation disclosed herein at both ambient and reduced temperatures. This benefit reduces the costs associated with the storage, transportation, and spoilage of a formulation, increases the convenience of storage and handling, and avoids the need to administer cold formulations, thereby improving subject tolerance and compliance to a regimen of a formulation of the disclosure.

The formulations can further include a pharmaceutically-acceptable excipient. Non-limiting examples of excipients include mannitol, sorbitol, lactose, dextrose, and cyclodextrins. Excipients can be added to modulate the density, rheology, uniformity, and viscosity of the formulation.

The formulations can include acidic or basic excipients to modulate the acidity or basicity of the formulation. Non limiting examples of acids suitable to increase the acidity of a formulation include hydrochloric acid, hydrobromic acid, hydroiodic acid, sulfuric acid, phosphoric acid, nitric acid, ascorbic acid, citric acid, tartaric acid, lactic acid, oxalic acid, formic acid, benzenesulphonic acid, benzoic acid, maleic acid, glutamic acid, succinic acid, aspartic acid, diatrizoic acid, and acetic acid. Non limiting examples of bases suitable to increase the basicity of a formulation include lithium hydroxide, sodium hydroxide, potassium hydroxide, sodium carbonate, sodium bicarbonate, sodium phosphate, potassium phosphate, sodium acetate, sodium benzoate, tetrabutylammonium acetate, tetrabutylammonium benzoate, and trialkyl amines. Polyfunctional excipients, such as ethylene diamine tetraacetic acid (EDTA), or a salt thereof, can also be used to modulate acidity or basicity.

A compound of Formula I as described herein can be present in a formulation in any amount. In some embodiments, the compound is present in a formulation at a concentration of 1 mg/mL to 130 mg/mL, 10 mg/mL to 130 mg/mL, 40 mg/mL to 120 mg/mL, 80 mg/mL to 110 mg/mL, about 1 mg/mL to about 130 mg/mL, about 10 mg/mL to about 130 mg/mL, about 40 mg/mL to about 120 mg/mL, or about 80 mg/mL to about 110 mg/mL. In some embodiments, the compound is present in a formulation at a concentration of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL. In some embodiments, the compound is present in formulation at a concentration of 100 mg/mL. In some embodiments, the compound is present in a formulation at a concentration of about 100 mg/mL.

The formulation can be prepared by contacting a compound described herein with a solvent or a mixture of solvents. Alternatively, the compound can be contacted with a single solvent, and other solvents can be added subsequently, as a mixture, or sequentially. When the final formulation is a solution, complete solvation can be achieved at whatever step of the process is practical for manufacturing. Optional excipients can be added to the formulation at whatever step is practical for manufacturing.

Preparation of a formulation disclosed herein can be optionally promoted by agitation, heating, or extension of the dissolution period. Non-limiting examples of agitation include shaking, sonication, mixing, stirring, vortex, and combinations thereof.

In some embodiments, a formulation disclosed herein is optionally sterilized. Non-limiting examples of sterilization techniques include filtration, chemical disinfection, irradiation, and heating.

Pharmaceutical compositions can be provided as part of a pharmaceutical kit or patient pack. Non-limiting examples of a pharmaceutical kit include an array of one or more unit doses of a pharmaceutical composition together with a dosing device (e.g. measuring device) and/or a delivery device (e.g. inhaler or syringe), optionally all contained within common outer packaging. In pharmaceutical kits comprising a combination of two or more compounds/agents, the individual compounds/agents can be unitary or non-unitary formulations. In some embodiments, the unit dose(s) can be contained within a blister pack. In some embodiments, the pharmaceutical kit further comprises instructions for use.

A patient pack can be a package, prescribed to a patient, which contains pharmaceutical compositions for the whole course of treatment. Patient packs can contain one or more blister pack(s). Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert improves patient compliance with the physician's instructions.

### Dosing and Administration.

Dosing can be determined using various techniques. In some embodiments, a dose can be selected to deliver a therapeutically-effective amount of a pharmaceutical agent such as a compound disclosed herein to a subject. The selected dose level can depend on a variety of factors, including but not limited to the activity of the compound employed, the route of administration, the time of administration, the rate of excretion of the compound, the duration of treatment, other drugs, compounds, and/or materials used in combination with the compound, the age, sex, weight, condition, general health, and prior medical history of the subject being treated, and the genotype (e.g. TP53 mutational status) of the subject being treated. The dosage values can also vary with the severity of the condition to be alleviated. For any particular subject, specific dosage regimens can be adjusted over time according to the individual need and the professional judgment of a person administering or supervising the administration of the compositions.

In some instances, the dose of an administered compound of the disclosure can be determined by reference to the plasma concentration of the administered compound. For example, the maximum plasma concentration (Cmax) and the area under the plasma concentration-time curve from time 0 to infinity (AUC) can be used.

Alternatively, the administered dosage of a compound disclosed herein to a subject can be based on the amount of the compound per body surface area (BSA) of the subject. Various formulas can be used to calculate the body surface area. Non-limiting examples of formulas that can be used to calculate body surface area are shown below in TABLE 1.

TABLE 1

| Formula Name | Formula* |
|---|---|
| Du Bois | BSA = 0.007184 × W^{0.425} × H^{0.725} |
| Mosteller | BSA = 0.016667 × W^{0.5} × H^{0.5} |
| Gehan and George | BSA = 0.0235 × W^{0.51456} × H^{0.42246} |
| Hakcock | BSA = 0.024265 × W^{0.5378} × H^{0.3964} |
| Shuter and Aslani | BSA = 0.00949 × W^{0.46} × H^{1.08} |
| Takahira | BSA = 0.007241 × W^{0.425} × H^{0.725} |
| Fujimoto | BSA = 0.008883 × W^{0.425} × H^{0.725} |
| Boyd | BSA = 0.03330 × W^{(0.6157-0.0188log10(W))} × H^{0.3} |

*BSA = Body surface area (m²), W = mass (kg), H = height (cm)

A therapeutically-effective amount of a compound disclosed herein can be from about 1 mg/m² to about 200 mg/mg². In some embodiments, the therapeutically-effective amount of a compound of the disclosure administered to the subject is about 1 mg/m² to about 10 mg/m², about 1 mg/m² to about 20 mg/m², about 1 mg/m² to about 30 mg/m², about 1 mg/m² to about 40 mg/m², about 1 mg/m² to about 50 mg/m², about 1 mg/m² to about 60 mg/m², about 1 mg/m² to about 70 mg/m², about 1 mg/m² to about 80 mg/m², about 1 mg/m² to about 90 mg/m², about 1 mg/m² to about 100 mg/m², about 1 mg/m² to about 200 mg/m², about 10 mg/m² to about 20 mg/m², about 10 mg/m² to about 30 mg/m², about 10 mg/m² to about 40 mg/m², about 10 mg/m² to about 50 mg/m², about 10 mg/m² to about 60 mg/m², about 10 mg/m² to about 70 mg/m², about 10 mg/m² to about 80 mg/m², about 10 mg/m² to about 90 mg/m², about 10 mg/m² to about 100 mg/m², about 10 mg/m² to about 200 mg/m², about 20 mg/m² to about 30 mg/m², about 20 mg/m² to about 40 mg/m², about 20 mg/m² to about 50 mg/m², about 20 mg/m² to about 60 mg/m², about 20 mg/m² to about 70 mg/m², about 20 mg/m² to about 80 mg/m², about 20 mg/m² to about 90 mg/m², about 20 mg/m² to about 100 mg/m², about 20 mg/m² to about 200 mg/m², about 30 mg/m² to about 40 mg/m², about 30 mg/m² to about 50 mg/m², about 30 mg/m² to about 60 mg/m², about 30 mg/m² to about 70 mg/m², about 30 mg/m² to about 80 mg/m², about 30 mg/m² to about 90 mg/m², about 30 mg/m² to about 100 mg/m², about 30 mg/m² to about 200 mg/m², about 40 mg/m² to about 50 mg/m², about 40 mg/m² to about 60 mg/m², about 40 mg/m² to about 70 mg/m², about 40 mg/m² to about 80 mg/m², about 40 mg/m² to about 90 mg/m², about 40 mg/m² to about 100 mg/m², about 40 mg/m² to about 200 mg/m², about 50 mg/m² to about 60 mg/m², about 50 mg/m² to about 70 mg/m², about 50 mg/m² to about 80 mg/m², about 50 mg/m² to about 90 mg/m², about 50 mg/m² to about 100 mg/m², about 50 mg/m² to about 200 mg/m², about 60 mg/m² to about 70 mg/m², about 60 mg/m² to about 80 mg/m², about 60 mg/m² to about 90 mg/m², about 60 mg/m² to about 100 mg/m², about 60 mg/m² to about 200 mg/m², about 70 mg/m² to about 80 mg/m², about 70 mg/m² to about 90 mg/m², about 70 mg/m² to about 100 mg/m², about 70 mg/m² to about 200 mg/m², about 80 mg/m² to about 90 mg/m², about 80 mg/m² to about 100 mg/m², about 80 mg/m² to about 200 mg/m², about 90 mg/m² to about 100 mg/m², about 90 mg/m² to about 200 mg/m², or about 100 mg/m² to about 200 mg/m². In some embodiments, the therapeutically-effective amount of a compound of the disclosure administered to the subject is about 1 mg/m², about 10 mg/m², about 20 mg/m², about 30 mg/m², about 40 mg/m², about 45 mg/m², about 50 mg/m², about 60 mg/m², about 70 mg/m², about 80 mg/m², about 90 mg/m², about 100 mg/m², or about 200 mg/m². In some embodiments, the therapeutically-effective amount of a compound of the disclosure administered to the subject is at least about 1 mg/m², about 10 mg/m², about 20 mg/m², about 30 mg/m², about 40 mg/m², about 45 mg/m², about 50 mg/m², about 60 mg/m², about 70 mg/m², about 80 mg/m², about 90 mg/m², or about 100 mg/m². In some embodiments, the therapeutically-effective amount of a compound of the disclosure administered to the subject is at most about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², or about 200 mg/m².

A therapeutically-effective amount of a compound disclosed herein can be administered according to varying dosing regimens. In some embodiments, the therapeutically-effective amount of the compound is administered once or multiple times per day. For example, a method of the disclosure can comprise administration of the compound 1, 2, 3, 4, 5, 6, 7, 8, 9, or 10 times per day. In some embodiments, the compound can be administered 1-35 times (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35), 1-14 times, or 1-7 times per week. In some embodiments, the compound can be administered 1-10 (i.e. 1, 2, 3, 4, 5, 6, 7, 8, 9, or 10) times per month.

A method of the disclosure can utilize a dosing regimen comprising an administration free period that occurs at a point in time between two administrations of a therapeutically-effective amount of a compound of the disclosure. For example, a dosing regimen disclosed herein can comprise administration of the compound, followed by an administration free period, followed by a second administration of the compound. An administration free period can last, for example, 1 day, 2 days, 3 days, 4 days, 5 days, 6 days 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 month, 2 months, or 3 months. In some embodiments, an administration free period can last at least 1 day, at least 2 days, at least 3 days, at least 4 days, at least 5 days, at least 6 days at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 1 month, at least 2 months, or at least 3 months. In some embodiments, an administration free period can last at most 1 day, at most 2 days, at most 3 days, at most 4 days, at most 5 days, at most 6 days at most 7 days, at most 8 days, at most 9 days, at most 10 days, at most 11 days, at most 12 days, at most 13 days, at most 14 days, at most 15 days, at most 16 days, at most 17 days, at most 18 days, at most 19 days, at most 20 days, at most 21 days, at most 22 days, at most 23 days, at most 24 days, at most 25 days, at most 26 days, at most 27 days, at most 28 days, at most 29 days, at most 1 month, at most 2 months, or at most 3 months.

Therapeutically-effective amounts of a compound of the disclosure can be administered in a treatment cycle. For example, administration of a therapeutically-effective amount of a compound disclosed herein can occur once per day on days 1-5 of a 28-day cycle. In some embodiments, a therapeutically-effective amount of a compound is administered on days 1-5 of a 28-day cycle and is not administered on days 6-28 of the 28-day cycle. In some embodiments, a treatment cycle can be repeated 2, 3, 4, 5, 6, 7, 8, 9, 10, or more than 10 times. In some embodiments, administration of a therapeutically-effective amount of a compound disclosed herein can occur once per day on days 1-5 of a 21-day cycle. In some embodiments, a therapeutically-effective amount of a compound is administered on days 1-5 of a 21-day cycle and is not administered on days 6-21 of the 21-day cycle. A treatment cycle can be repeated at least 2, at least 3, at least 4, at least 5, at least 6, at least 7, at least 8, at least 9, or at least 10 times. In some embodiments, administration of a minimum number of treatment cycles to a subject can increase the probability that the treatment is effective compared to treatment of a subject that does not receive the minimum number of treatment cycles.

Compounds of the present disclosure can be administered by various routes. Non-limiting examples of administration routes include intravenous, subcutaneous, intramuscular, oral, rectal, intraocular, aerosol, parenteral, ophthalmic, pulmonary, transdermal, vaginal, otic, nasal, and topical administration.

In some embodiments, a compound described herein or a pharmaceutically-acceptable salt thereof can be administered to a subject subcutaneously and can provide prolonged in vivo exposure to the active metabolite of the compound. For example, administration of Compound I-1 or a pharmaceutically-acceptable salt thereof can provide prolonged in vivo exposure of the active metabolite of I-1, decitabine.

### Therapeutic Uses

A method of the present disclosure can be used to treat cancer. In some embodiments a method disclosed herein is used to treat benign tumors or malignant tumors. Generally, cells in a benign tumor retain differentiated features and do not divide in a completely uncontrolled manner. A benign tumor is usually localized and nonmetastatic.

In a malignant tumor, cells become undifferentiated, do not respond to growth control signals, and multiply in an uncontrolled manner. Malignant tumors are invasive and capable of metastasizing. The common routes for metastasis are direct growth into adjacent structures, spread through the vascular or lymphatic systems, and tracking along tissue planes and body spaces (e. g. peritoneal fluid or cerebrospinal fluid).

Types of cancers that can be treated using a method of the disclosure include, for example, breast cancer, skin cancer, bone cancer, prostate cancer, liver cancer, lung cancer, brain cancer, cancer of the larynx, cancer of the gall bladder, pancreatic cancer, rectal cancer, parathyroid cancer, thyroid cancer, adrenal cancer, neural tissue cancer, head and neck cancer, colon cancer, stomach cancer, cancer of the bronchi, renal cancer, basal cell carcinoma, squamous cell carcinoma of both ulcerating and papillary type, metastatic skin carcinoma, osteosarcoma, Ewing's sarcoma, veticulum cell sarcoma, myeloma, giant cell tumor, small-cell lung tumor, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wïlm's tumor, seminoma, ovarian tumor, cervical dysplasia and in situ carcinoma, neuroblastoma, retinoblastoma, soft tissue sarcoma, malignant carcinoid, mycosis fungoide, rhabdomyosarcoma, Kaposi's sarcoma, osteogenic and other sarcoma, renal cell tumor, polycythemia vera, adenocarcinoma, glioblastoma multiforma, leukemias, lymphomas, malignant melanomas, epidermoid carcinomas, carcinomas, sarcomas, hemangiomas, hepatocellular adenoma, cavernous hemangioma, focal nodular hyperplasia, acoustic neuromas, neurofibroma, bile duct adenoma, bile duct cystanoma, fibroma, lipomas, leiomyomas, mesotheliomas, teratomas, myxomas, and nodular regenerative hyperplasia.

In some embodiments, a method of the present disclosure can be used to treat, for example, myelodysplastic syndromes (MDS). MDSs are heterogeneous clonal hematopoietic stem cell disorders associated with the presence of dysplastic changes in one or more of the hematopoietic lineages, including dysplastic changes in the myeloid, erythroid, and megakaryocytic series. These changes can result in cytopenias in one or more of the three lineages. Subjects afflicted with MDS typically develop complications related to anemia, neutropenia (infections), or thrombocytopenia (bleeding). Generally, from about 10% to about 70% of subjects with MDS develop acute leukemia. Non-limiting examples of myelodysplastic syndromes include acute myeloid leukemia, acute promyelocytic leukemia, acute lymphoblastic leukemia, and chronic myelogenous leukemia. In some embodiments, a compound disclosed herein can treat MDS.

Acute myeloid leukemia (AML) is the most common type of acute leukemia in adults. Several inherited genetic disorders and immunodeficiency states are associated with an increased risk of AML. The disorders include those with defects in DNA stability leading to random chromosomal breakage, such as Bloom's syndrome, Fanconi's anemia, Li-Fraumeni kindreds, ataxia-telangiectasia, and X-linked agammaglobulinemia. In some embodiments, a compound disclosed herein can treat AML.

Acute promyelocytic leukemia (APML) represents a distinct subgroup of AML. This subtype is characterized by promyelocytic blasts containing the 15; 17 chromosomal translocation. This translocation leads to the generation of a fusion transcript comprising a retinoic acid receptor sequence and a promyelocytic leukemia sequence. In some embodiments, a compound disclosed herein can treat APML.

In some embodiments, a compound disclosed herein can treat Acute lymphoblastic leukemia (ALL). ALL is a heterogeneous disease with distinct clinical features displayed by various subtypes. Reoccurring cytogenetic abnormalities have been demonstrated in ALL. The most common associated cytogenetic abnormality is the 9; 22 translocation leading to development of the Philadelphia chromosome.

In some embodiments, a compound disclosed herein can treat chronic myelogenous leukemia (CML). CML is a clonal myeloproliferative disorder of a pluripotent stem cell, generally caused by ionizing radiation. CIVIL is characterized by a specific chromosomal abnormality involving the translocation of chromosomes 9 and 22, creating the Philadelphia chromosome.

### Mechanism of Therapy.

Compounds of the present disclosure can treat cancer via various mechanisms. In some embodiments, the compounds of the present disclosure can be used to control intracellular gene expression. DNA methylation is associated with the control of gene expression. Specifically, methylation in or near promoters inhibit transcription while demethylation restores expression. The compounds disclosed herein can reverse aberrant DNA hypermethylation in genes including, for example, tumor suppressor genes via inhibition of DNMT.

In some embodiments, a compound disclosed herein can reverse aberrant hypermethylation through the release of an active metabolite that forms as a result of enzymatic degradation of the compound. For example, a phosphodiester bond of a compound disclosed herein can be enzymatically cleaved in vivo to release decitabine. In cells, decitabine is converted into an active form, the phosphorylated 5-aza-deoxycytidine, by deoxycytidine kinase, which is primarily synthesized during the S phase of the cell cycle. The affinity of decitabine for the catalytic site of deoxycytidine kinase is similar to the natural substrate, deoxycytidine. After conversion to a triphosphate form by deoxycytidine kinase, decitabine is incorporated into replicating DNA at a rate similar to that of the natural substrate, dCTP.

After chromosomal duplication, 5-methylcytosine residues on parent DNA strands direct methylation on complementary daughter DNA strands in a process catalyzed by DNMTs. However, the presence of decitabine in DNA strands interferes with this normal process of DNA methylation due to the presence of nitrogen at the C-5 position of decitabine. The replacement of cytosine with decitabine at a specific site of methylation produces an irreversible inactivation of DNMTs. Decitabine behaves as a cytosine residue until DNMT enzymes attempt to transfer a methyl group to the hemimethylated DNA strands of the daughter cells. At this step, the DNMT enzyme is covalently trapped by decitabine in the DNA, and cannot further methylate additional cytosine residues. By reducing the likelihood of methylation, decitabine allows genes silenced via methylation from previous rounds of cell division to be re-expressed. The active trap is present in the hemimethylated DNA up to 48 hours after decitabine treatment. After further DNA synthesis and cell cycle division, progeny strands from the hemimethylated DNA result in DNA strands that are completely unmethylated at these sites. By specifically inhibiting DNMTs, aberrant methylation of genes including, for example, tumor suppressor genes can be reversed.

In some embodiments, formation of decitabine via the enzymatic degradation of a compound disclosed herein can prolong a subject's exposure window to decitabine and result in more efficient inhibition of DNMT compared to direct administration of decitabine. Moreover, the prolonged exposure window of decitabine can result in better access to target tissues including, for example, bone marrow.

A method disclosed herein can comprise administering a compound of the present disclosure to a subject suffering from a disease associated with abnormal levels of gene expression. Examples of the possible applications of the described mechanisms include, but are not limited to, therapeutically modulated growth inhibition, induction of apoptosis, and cell differentiation. In some embodiments, a compound disclosed herein modulates a target in the p53 pathway. Non-limiting examples of targets in the p53 pathway include AKT1, AKT2, AKT3, ALK, BRAF, CDK4, CDKN2A, DDR2, EGFR, ERBB2 (HER2), FGFR1, FGFR3, GNA11, GNQ, GNAS, KDR, KIT, KRAS, MAP2K1 (MEK1), MET, HRAS, NOTCH1, NRAS, NTRK2, PIK3CA, NF1, PTEN, RAC1, RB1, NTRK3, STK11, PIK3R1, TSC1, TSC2, RET, TP53, and VHL. In some embodiments, a compound disclosed herein modulates a target in an apoptotic pathway.

Gene activation facilitated by the compounds of the present disclosure can induce differentiation of cells for therapeutic purposes. Cellular differentiation can be induced through the mechanism of hypomethylation. Examples of morphological and functional differentiation include, but are not limited to, differentiation towards formation of muscle cells, myotubes, cells of erythroid and lymphoid lineages.

### Combination Therapies.

A compound of the disclosure such as a DHA, can be used in combination with an additional therapeutic agent to treat cancer. In some embodiments, a combination therapy with a compound of the disclosure and an additional therapeutic agent can produce a more efficacious therapeutic result than the additive effects achieved by each individual constituent when administered alone at a therapeutic dose. In some embodiments, the dosage of a DHA or an additional therapeutic agent can be reduced as compared to monotherapy with each agent, while still achieving an overall therapeutic effect. In some embodiments, a DHA and an additional therapeutic agent, for example, any additional therapeutic agent described herein, can exhibit a synergistic effect. In some embodiments, the synergistic effect of a DHA and an additional therapeutic agent, for example, and additional therapeutic agent disclosed herein, can be used to reduce the total amount of drugs administered to a subject. In some embodiments, a combination therapy with a DHA and an additional therapeutic agent can decrease an adverse effect associated with the DHA or the additional therapeutic agent. For example, a combination therapy disclosed herein can reduce neutropenia or thrombocytopenia associated with administration of a compound disclosed herein. Non-limiting examples of additional therapeutic agents that can be used in combination with a DHA include anti CTLA-4 antibodies such as ipilimumab and tremelimumab; alykylating antineoplastic agents such as cyclophosphamide and cisplatin; PD1 and PD-L1 inhibitors such as nivolumab, pembrolizumab, atezolizumab, avelumab, durvalumab, and cemiplimab.

### Assessment of Therapeutic Efficacy.

The therapeutic efficacy of treatments disclosed herein can be assessed using various criteria. For example, a clinical trial can be performed to determine the complete response (or remission) (CR) rate and overall survival (OS) of subjects receiving a treatment disclosed herein. A subject is determined to have a CR upon the disappearance of all signs of cancer. The CR rate indicates the proportion of patients receiving a treatment that exhibited a CR. OS is the length of time that subjects diagnosed with a disease remain alive following either diagnosis of the disease or the initiation of treatment. In some embodiments, therapeutic efficacy can be assessed based on marrow complete response (mCR). A patient can be said to have exhibited a mCR upon reduction to ≤5% myeloblasts and a decrease of persistent cytopenias of ≥50% compared to baseline.

The therapeutic efficacy of a treatment disclosed herein can also be assessed using the EQ-5D instrument. EQ-5D is a standardized instrument for use as a measure of health outcome frequently employed in clinical trials. EQ-5D is designed for self-completion by a trial participant via a questionnaire. An EQ-5D questionnaire has two components: health state description and evaluation. In the health state description component, health status is measured in terms of mobility, self-care, usual activities, pain-discomfort, and anxiety/depression. The mobility dimension asks about a person's walking ability. The self-care dimension asks about the ability to wash or dress by oneself, and the usual activities dimension measures performance work, study, housework, family, or leisure activities. In the pain/discomfort and anxiety/depression dimensions, participants are asked about their levels of pain/discomfort and anxiety/depression. In an EQ-5D-5L version of a EQ-5D questionnaire, respondents rate their level of severity for each dimension using a five-level scale. In the evaluation component of an EQ-5D questionnaire respondents answer using a visual analogue scale (EQ-VAS). Respondents mark their health status on a 20 cm vertical scale with end points of 0 and 100. Notes are present on both ends of the scale so that 0 corresponds to “the worst health you can imagine” and 100 corresponds to “the best health you can imagine”.

Overall health of a subject can also be assessed before or after treatment with a compound disclosed herein via Eastern Cooperative Oncology Group (ECOG) performance status (PS). The ECOG performance status assigns a 0-5 score to a subject based on the criteria shown below in TABLE 2.

TABLE 2

| GRADE | ECOG PERFORMANCE STATUS |
|---|---|
| 0 | Fully active, able to carry on all pre-disease |
| | performance without restriction |
| 1 | Restricted in physically strenuous activity but |
| | ambulatory and able to carry out work of a light |
| | or sedentary nature, e.g., light house work, |
| | office work |
| 2 | Ambulatory and capable of all selfcare but unable |
| | to carry out any work activities; up and about more |
| | than 50% of waking hours |
| 3 | Capable of only limited selfcare; confined to bed |
| | or chair more than 50% of waking hours |
| 4 | Completely disabled; cannot carry on any selfcare; |
| | totally confined to bed or chair |
| 5 | Dead |

A landmark survival analysis can also be used to assess the efficacy of a treatment disclosed herein. A landmark survival analysis designates a timepoint during the follow up period of a clinical investigation (known as the landmark time) and analyzes only those subjects who have survived until the landmark time. Non-limiting examples of landmark times include about 1 week, about 2 weeks about 3 weeks, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at most about 1 week, at most about 2 weeks, at most about 3 weeks, at most about 1 month, at most about 2 months, at most about 3 months, at most about 4 months, at most about 5 months, at most about 6 months, at most about 7 months, at most about 8 months, at most about 9 months, at most about 10 months, at most about 11 months, at most about 1 year, at most about 2 years, at most about 3 years, at most about 4 years, and at most about 5 years. In some embodiments, a landmark survival analysis can be used to determine a minimum number of treatments or treatment cycles that is needed to increase the probability of efficacy of the treatment.

In some embodiments, the International Prognostic Scoring System (IPSS) can be used to classify subjects based on disease risk prior to treatment with a compound of the disclosure. IPSS uses three prognostic indicators to predict the course of a subject's disease: the percentage of blasts cells in the marrow (bone marrow blasts), the type of chromosomal changes (if any) in marrow cells, and the presence of one or more low blood counts (cytopenias). Based off of these indicators, a score can be assigned to a subject with a higher score indicating a subject at higher risk for disease progression.

The therapeutic efficacy of a treatment disclosed herein can be assessed by how long the treatment prolongs survival of the patient, or how long the treatment is expected to prolong survival of a patient. In some embodiments a treatment disclosed herein prolongs survival of a patient by at least about 1 week, at least about 1 month, at least about 2 months, at least about 3 months, at least about 4 months, at least about 5 months, at least about 6 months, at least about 1 year, or at least about 2 years. In some embodiments, a treatment disclosed herein prolongs (or is expected to prolong) survival of a patient that is assigned to a subgroup of patients longer than the treatment prolongs (or is expected to prolong) survival in a patient that does not belong to the subgroup.

### Examples

### Example 1: Phase 1/2 Trial (Phase 2 Portion)

A multicenter, open-label, randomized, phase 1/2 trial was performed with Compound I-1 to compare the activity and safety of two doses of Compound I-1 in hypomethylating agent treatment naïve or relapsed or refractory patients with intermediate-risk or high-risk myelodysplastic syndromes.

Diagnosis and Eligibility Criteria:

Patients enrolled in the study were aged 18 years or older, with a confirmed diagnosis of IPSS intermediate-1, intermediate-2, or high-risk myelodysplastic syndromes (MDS), or chronic myelomonocytic leukemia (CMML). Patients were either hypomethylating agent treatment-naïve or had relapsed or refractory disease after previous hypomethylating agent treatment as determined by the investigators' judgment (two or more complete full-dose cycles of a hypomethylating agent). The IPSS score in patients with relapsed or refractory myelodysplastic syndromes had been established at the time of their initial diagnosis. Eligible patients had Eastern Cooperative Oncology Group performance status of 0-2; adequate renal function (serum creatinine ≤1.5-times the upper limit of normal [ULN]) and adequate hepatic function (total bilirubin ≤2-times the ULN, and aspartate and alanine transaminases ≤2.5-times the ULN); had not undergone major surgery within 4 weeks or hemopoietic stem-cell transplantation within 8 weeks; or received chemotherapy within 2 weeks or nitrosoureas within 6 weeks of Compound I-1 treatment (hydroxycarbamide was allowed during treatment cycle 1). A patient with previous allogeneic stem-cell transplants was only eligible if the patient had no evidence of active graft-versus-host disease and had discontinued immunosuppressive therapy 2 weeks or more before receiving the study drug.

Patients with acute promyelocytic leukemia, previous malignancy (except for adequately treated basal cell or squamous cell skin cancer, in-situ cervical cancer, or other cancers from which the patient had been disease free for ≥3 years), life-threatening illness other than myelodysplastic syndromes or acute myeloid leukemia, symptomatic arrhythmias, New York Heart Association class 3 or 4 heart disease, symptomatic CNS metastases, known HIV infection, or active infection with hepatitis B or C virus were excluded. Patients with grade 2 or worse toxicity (using Common Terminology Criteria for Adverse Events version 4.0) from previous therapy (except for alopecia), individuals who had been given any investigational drug within 2 weeks of randomization, individuals who received radiotherapy for extramedullary disease within 2 weeks, and individuals with treatment concurrent with systemic corticosteroids for myelodysplastic syndromes were also excluded.

Randomization and Masking:

Randomization was done centrally using a stratified, dynamic randomization process data management system to assign patients randomly (1:1) to receive subcutaneous Compound I-1 at either 60 or 90 mg/m². Treatment allocation was stratified by disease status: hypomethylating agent treatment-naïve or relapsed or refractory (previous treatment with hypomethylating agent). Randomization was dynamic (i.e. the probability of assigning a patient to a dose group was dependent on the number of patients already randomized to each group in the overall trial). Treatment assignment information was communicated to the clinical sites at the time of patient randomization. The trial was open-label and no masking was involved. Patients were enrolled by investigators and other site staff, who continued to be involved in clinical care.

Procedures:

Patients received subcutaneous Compound I-1 at 60 or 90 mg/m² on days 1-5 of a 28-day treatment cycle. The intention was to provide the standard planned dose for four or more cycles; dose reduction could be instituted, as necessary, and the length of treatment cycles could be extended up to 42 days to allow for bone-marrow recovery in cases of severe cytopenia. Additional delay or dose reduction was allowed for recovery from toxicity in previous cycles based on the physician's judgment.

Hematological responses were monitored by analysis of blood and bone marrow aspiration. After the initial bone marrow aspirate screening at baseline, the results of peripheral blood assessments determined the frequency of subsequent bone marrow aspiration to confirm response or assess drug-related bone marrow toxicity. Complete peripheral blood counts and white blood cell differentials were measured at least once a week, including granulocyte numbers, platelet numbers, and hemoglobin concentration.

Safety was monitored throughout the study by physical examinations and clinical laboratory tests, including hematology, chemistry for liver and renal function, urinalysis, pregnancy tests, pharmacokinetics, epi-genetics, buccal swab, and pharmacogenetic markers. Electrocardiograms were obtained at baseline screening and day 1 of each treatment cycle.

Whole-blood samples were collected immediately before treatment, once a week during the first treatment cycle, and then on day 1 of subsequent cycles for demethylation analysis. Global DNA methylation was measured by the long interspersed nuclear element-1 (LINE-1) methylation assay. The LINE-1 assay measures methylation levels of LINE-1 repeats, which serve as a surrogate for global DNA methylation levels. Results of LINE-1 assays were used to assess and changes in methylation from baseline.

Study Endpoints:

The primary endpoint was overall response, which was a composite of complete response (CR), partial response, marrow complete response (mCR), and hematological improvement. Overall response was assessed by local investigators. Secondary endpoints included the individual components of overall response, time to response, duration of response, overall survival, blood transfusion and platelet transfusion independence at weeks 8 and 16, and safety, including the incidence of adverse events, and all-cause early mortality at 30, 60, and 90 days. Exploratory analyses were done to examine the association between baseline characteristics and overall response.

Statistical Analysis:

Initially, a minimum of 30 patients were to be enrolled in each treatment group (treatment-naïve and relapsed or refractory myelodysplastic syndrome groups). This sample size was selected such that if no responses were observed, the study could determine with 95% confidence that the proportion of patients with a response was less than 10% and further evaluation of that dose was not warranted. The protocol allowed a safety review committee to expand the number of participants in either or both groups to 50 if justified by promising activity and safety data. Because overall responses were seen at both doses, enrolment continued to approximately 50 patients per dose level. These criteria were pre-specified.

Activity and safety endpoints were evaluated for all patients who received at least one dose of study drug, and no patients who received treatment were excluded from the analyses. The proportion of patients who had an overall response represents the number of patients achieving an overall response divided by the total number of patients; the 95% CI of the proportion was based on binomial distribution. Comparison of the proportion of patients who had an overall response between the Compound I-1 60 and 90 mg/m² groups was made using Fisher's exact test. Time to response and duration of response were summarized in days using descriptive statistics. Overall survival was analyzed using the Kaplan-Meier method, with survival time censored on the last date of contact if the patient was alive or lost to follow-up. Blood and platelet transfusion independence were summarized descriptively in the subset of patients who were transfusion-dependent at baseline. Change from baseline LINE-1 methylation and maximum LINE-1 demethylation were also summarized descriptively, with maximum LINE-1 demethylation defined as the largest percent decrease from baseline in methylation values by patient between days 8 and 22 of the first treatment cycle.

Treatment-emergent adverse events, which are events that first occurred or worsened after the first dose of study drug until 30 days after the last dose or until the start of an alternative anticancer treatment (whichever occurred first), and all-cause 30-day, 60-day, and 90-day cumulative mortality, were summarized descriptively.

Subject Disposition:

105 patients were enrolled, of whom 51 were hypomethylating agent treatment-naïve and 54 had relapsed or refractory disease. Overall, 55 patients were randomly assigned to Compound I-1 60 mg/m² and 50 patients were randomly allocated to Compound I-1 90 mg/m². Three patients did not receive Compound I-1 (one patient who was treatment-naïve and one patient with relapsed or refractory disease allocated to the 60 mg/m² group, and one patient who was treatment-naïve allocated to the 90 mg/m² group) and were not included in subsequent analyses.

Median follow-up was 3.2 years (interquartile range [IQR] 2.8-3.5) for the entire study population. Despite the long-term follow-up in this study, 22 patients were still alive at the time of the database lock, including five patients who continued to receive study treatment and eight patients who underwent or planned to undergo hemopoietic stem-cell transplantation.

Patient demographics and baseline characteristics are summarized in TABLE 3. Median patient age was 72 years (range 18-89 years), with similar demographic characteristics between Compound I-1 dose groups and disease cohorts. Among patients who were hypomethylating agent treatment-naïve, baseline characteristics were generally well balanced between Compound I-1 dose groups, although numerically fewer patients in the 60 mg/m² group had a bone-marrow blast percentage of more than 5% compared to the 90 mg/m² group. As expected, the relapsed or refractory cohort had a longer disease duration, and higher proportions of patients with high-risk myelodysplastic syndromes and bone-marrow blast percentages of more than 5% than the hypomethylating agent treatment-naïve cohort. Among patients with relapsed or refractory disease, baseline characteristics were also generally balanced between Compound I-1 groups. However, the 60 mg/m² group had a higher proportion of patients with CMML, and lower proportions with high-risk myelodysplastic syndromes and bone-marrow blast percentages of more than 5%. 51 (96%) of 53 patients with relapsed or refractory disease had received previous hypomethylating agent treatment. 30 (57%) patients had received the treatment within the preceding 3-month period and 41 (77%) for an adequate duration of therapy of 6 months or more. In addition to hypomethylating agents, previous treatment regimens included lenalidomide, other cytotoxic agents, and supportive drugs. Overall, 58 (57%) of 102 patients were red blood (RBC) transfusion-dependent and 28 (27%) were platelet transfusion-dependent.

TABLE 3

| | DHA treatment-naive MDS | | | | Relapsed or refractory MDS | | | |
|---|---|---|---|---|---|---|---|---|
| | Compound I-1 | | Compound I-1 | | Compound I-1 | | Compound I-1 | |
| | 60 mg/m² | | 90 mg/m² | | 60 mg/m² | | 90 mg/m² | |
| | (n = 27) | | (n = 22) | | (n = 26) | | (n = 27) | |
| Age (years) | 72 | (18-85) | 71 | (64-85) | 73 | (55-86) | 72 | (52-89) |
| Sex | | | | | | | | |
| Men | 21 | (78%) | 14 | (64%) | 16 | (62%) | 16 | (59%) |
| Women | 6 | (22%) | 8 | (36%) | 10 | (38%) | 11 | (41%) |
| Ethnic origin* | | | | | | | | |
| White | 25 | (93%) | 22 | (100%) | 25 | (96%) | 25 | (93%) |
| Asian | 1 | (4%) | 0 | | 1 | (4%) | 1 | (4%) |
| Black | 0 | | 0 | | 0 | | 1 | (4%) |
| ECOG performance status | | | | | | | | |
| 0 | 6 | (22%) | 7 | (32%) | 6 | (23%) | 5 | (19%) |
| 1 | 19 | (70%) | 14 | (64%) | 14 | (54%) | 17 | (63%) |
| 2 | 2 | (7%) | 1 | (5%) | 6 | (23%) | 5 | (19%) |
| IPSS classification | | | | | | | | |
| Intermediate-1 risk | 14 | (52%) | 9 | (41%) | 2 | (8%) | 2 | (7%) |
| Intermediate-2 risk | 1 | (4%) | 4 | (18%) | 6 | (23%) | 7 | (26%) |
| High risk | 6 | (22%) | 3 | (14%) | 9 | (35%) | 16 | (59%) |
| CMML | 6 | (22%) | 6 | (27%) | 9 | (35%) | 1 | (4%) |
| Not evaluable | 0 | | 0 | | 0 | | 1 | (4%) |
| Time since diagnosis | 35 | (6-2257) | 34 | (3-2237) | 727 | (26-3090) | 466 | (15-3202) |
| (days) | | | | | | | | |
| Previous regimens | 0 | (0-1) | 0 | (0-1) | 1 | (1-4) | 1 | (1-4) |
| (number)^{$} | | | | | | | | |
| Previous DHA | 1 | (4%) | 0 | | 24 | (92%) | 27 | (100%) |
| (decitabine or | | | | | | | | |
| azacytidine) | | | | | | | | |
| Time since last DHA treatment | | | | | | | | |
| (% of those with previous DHA) | | | | | | | | |
| <3 months | 1/1 | (100%) | NA | | 16/24 | (67%) | 14/27 | (52%) |
| ≥3 months | 0/1 | | NA | | 8/24 | (33%) | 13/27 | (48%) |
| Duration of previous DHA treatment | | | | | | | | |
| (% of those with previous DHA) | | | | | | | | |
| <6 months | 1/1 | (100%) | NA | | 3/24 | (13%) | 7/27 | (26%) |
| >6 months | 0/1 | | NA | | 21/24 | (88%) | 20/27 | (74%) |
| Bone-marrow blasts | 2 | (0-13) | 7 | (0-14) | 6 | (0-18) | 9 | (1-19) |
| ≤5% | 20 | (74%) | 10 | (45%) | 13 | (50%) | 6 | (22%) |
| >5% | 7 | (26%) | 12 | (55%) | 13 | (50%) | 21 | (78%) |
| Peripheral blood blasts | 0 | (0-13) | 0 | (0-6) | 0 | (0-32) | 0 | (0-21) |
| Platelets | 54 | (12-424) | 73 | (9-1202) | 39 | (15-328) | 35 | (7-210) |
| (×109 per L) | | | | | | | | |
| Neutrophils (x 109 per L) | 1.2 | (0.2-36.9) | 2.6 | (0.8-164) | 1.2 | (0.1-13.3) | 0.5 | (0.1-15.6) |
| Hemoglobin (g/dL) | 9.3 | (6.9-16.4) | 9.0 | (7.7-12.3) | 9.3 | (7.1-12.9) | 9.5 | (74415) |
| RBC transfusion- | 15 | (56%) | 9 | (41%) | 16 | (62%) | 18 | (67%) |
| dependent | | | | | | | | |
| Platelet transfusion- | 7 | (26%) | 5 | (23%) | 6 | (23%) | 10 | (37%) |
| dependent | | | | | | | | |

Data are median (range) or n (%) unless otherwise specified. MDS-myelodysplastic syndrome. ECOG-Eastern Cooperative Oncology Group. IPSS-International Prognostic Scoring System. CMML-chronic myelomonocytic leukemia. RBC-red blood cell. NA-not applicable.
*Ethnic origin data missing for one individual.
^{$}All 53 patients with relapsed or refractory disease received previous therapy for MDS; all received previous DHA treatment except for one patient with CMML who received multiple cycles of ruxolitinib, and one with high-risk MDS who received multiple cycles of rigosertib and lenalidomide. One patient in the DHA treatment-naive cohort received one cycle of decitabine, consistent with the eligibility criteria for this cohort.

The median number of treatment cycles was 5 (range 1-49) in the Compound I-1 60 mg/m² group and 4.5 (range 1-41) in the Compound I-1 90 mg/m² group among patients who were hypomethylating agent treatment-naïve. 13 (48%) patients who were treatment-naïve receiving Compound I-1 60 mg/m² and 10 (45%) patients who were treatment-naïve receiving Compound I-1 90 mg/m² went on to receive six or more cycles of therapy. Likewise, 9 (35%) patients with relapsed or refractory disease on the 60 mg/m² dose and 12 (44%) patients with relapsed or refractory disease on the 90 mg/m² dose received the drug for six or more cycles. 95 (93%) patients received at least 90% of the planned total dose in the treatment cycles received. Regardless of dose, the proportions of patients with treatment delays generally increased over time in both patients who were treatment-naïve (from 14 [32%] of 44 in cycle 2 to 13 [52%] of 25 in cycle 5) and patients with relapsed or refractory disease (from 18 [38%] of 47 in cycle 2 to 15 [56%] of 27 in cycle 5). The proportions of patients with dose reductions also increased over time (from four [9%] of 44 in cycle 2 to seven [28%] of 25 in cycle 5 in the treatment-naïve cohort and from seven [15%] of 47 to 15 [56%] of 27 in the relapsed or refractory cohort). The proportions of dose delays and reductions were generally similar between the two Compound I-1 doses. Duration of Compound I-1 exposure was longer in the hypomethylating agent treatment-naïve cohort than in the relapsed or refractory cohort.

A comparison of treatment duration, dose reductions, and dose delays for treatment naïve and relapsed refractory MDS patients are shown in TABLE 4.

TABLE 4

| Treatment | Prev. Treated MDS | | Treatment naive MDS | |
|---|---|---|---|---|
| Median # cycles (range) | 5 | (1-37) | 5 | (1-49) |
| Treatment Duration: | 21 | (40%) | 23 | (47%) |
| >6 cycles | | | | |

| | | |
|---|---|---|
| % of delayed Cycles | 47% | 35% |
| % of Dose-reduced cycles | 34% | 37% |

Efficacy Results:

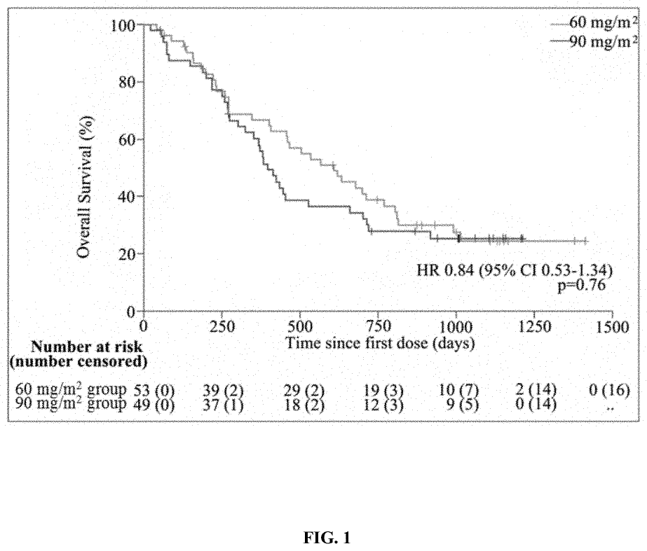

Efficacy results are summarized below in TABLE 5. The primary endpoint of overall response was achieved by 48 (47%; 95% CI 37-57) of 102 patients who received Compound I-1, including 21 (40%; 27-54) of 53 patients in the 60 mg/m² group and 27 (55%; 40-69) of 49 patients in the 90 mg/m² group. The difference in the proportions of patients who had an overall response between dose levels was not statistically significant (p=0.16). Similarly, a statistically significant difference was not observed in the OS between dose levels as seen in FIG. 1 (p=0.76). Complete response was similar between groups, occurring in six (11%) of 53 patients in the 60 mg/m² group and seven (14%) of 49 patients in the 90 mg/m² group. Hematological improvement was similar between groups. More patients in the 90 mg/m² group than the 60 mg/m² group had a marrow complete response, possibly reflecting the greater proportion of patients in that group with baseline bone-marrow blast percentages of more than 5% that could qualify for a marrow complete response or, perhaps, more potent effects in the bone marrow seen with the larger dose.

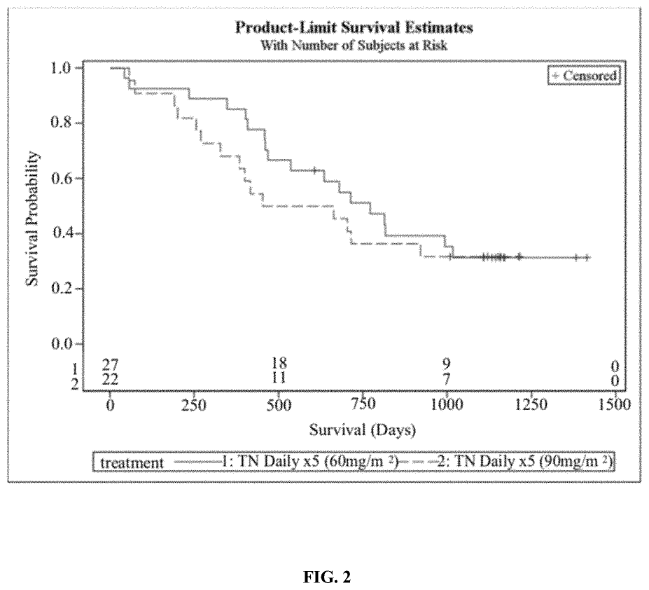

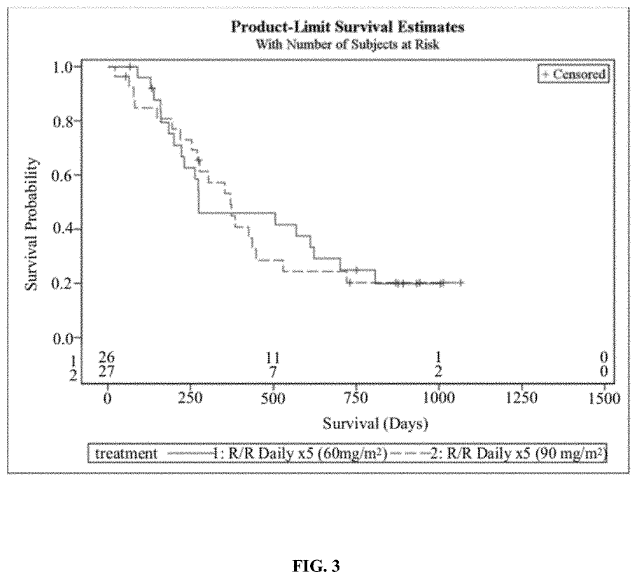

By disease cohort, overall response was achieved by 25 (51%, 95% CI 36-66) of 49 patients who were hypomethylating agent treatment-naïve and by 23 (43%, 95% CI 30-58) of 53 patients with relapsed or refractory disease. A complete response with Compound I-1 was reached in 11 (22%) patients who were treatment-naïve and two (4%) patients with relapsed or refractory disease. Further results on response rates and overall survival in treatment naïve and relapsed or refractory patients can be seen in TABLE 5, FIG. 2, and FIG. 3.

In an exploratory analysis, the proportions of patients who had an overall response were similar between patients with CMLL (10 [45%] of 22) and myelodysplastic syndromes (38 [48%] of 80).

TABLE 5

| | Compound I-1 dose | | | | Disease cohort | | | | | |
|---|---|---|---|---|---|---|---|---|---|---|
| | 60 | | 90 | | Treatment- | | Relapsed or | | All | |
| | mg/m² | | mg/m² | | naïve | | refractory | | patients | |
| | (n = 53) | | (n = 49) | | (n = 49) | | (n = 53) | | (n = 102) | |
| Overall | 21 (40%; 26.5-54.0) | | 27 (55%; 40.2-69.3) | | 25 (51%; 36.3-65.6) | | 23 (43%; 29.8-57. 7) | | 48 (47%; 37.1-57.2) | |
| response* | | | | | | | | | | |
| Complete | 6 | (11%) | 7 | (14%) | 11 | (22%) | 2 | (4%) | 13 | (13%) |
| response | | | | | | | | | | |
| Partial | 0 | | 0 | | 0 | | 0 | | 0 | |
| response | | | | | | | | | | |
| Marrow | 6 | (11%) | 16 | (33%) | 7 | (14%) | 15 | (28%) | 22 | (22%) |
| complete | | | | | | | | | | |
| response | | | | | | | | | | |
| HI | 18 | (34%) | 18 | (37%) | 21 | (43%) | 15 | (28%) | 36 | (35%) |
| HI-neutrophil | 7 | (13%) | 6 | (12%) | 7 | (14%) | 6 | (11%) | 13 | (13%) |
| HI-platelet | 10 | (19%) | 13 | (27%) | 13 | (27%) | 10 | (19%) | 23 | (23%) |
| Hi-erythroid | 10 | (19%) | 8 | (16%) | 13 | (27%) | 5 | (9%) | 18 | (18%) |
| Single-lineage | 11 | (21%) | 10 | (20%) | 11 | (22%) | 10 | (19%) | 21 | (21%) |
| HI | | | | | | | | | | |
| Bilineage HI | 5 | (9%) | 7 | (14%) | 8 | (16%) | 4 | (8%) | 12 | (12%) |
| Trilineage HI | 2 | (4%) | 1 | (2%) | 2 | (4%) | 1 | (2%) | 3 | (3%) |
| No response | 29 | (55%) | 20 | (41%) | 20 | (41%) | 29 | (55%) | 49 | (48%) |
| Not evaluable | 3 | (6%) | 2 | (4%) | 4 | (8%) | 1 | (2%) | 5 | (5%) |

Data are n (%; 95% CI) or n (%). Responses are based on modified 2006 International Working Group Response Criteria in Myelodysplasia. HI = hematological improvement
*Indicates a complete response, partial response, marrow complete response, or HI. Some patient in the HI category have also been counted in one of the other response categories (i.e. complete, partial, or marrow complete response). Patients were counted only once for overall response.

The maximum extent of global DNA demethylation measured by LINE-1 methylation analysis occurred around day 8 and then returned to pretreatment levels by day 28. The mean maximum LINE-1 demethylation with Compound I-1 was greater for patients receiving the 90 mg/m² dose (28.2%, SE 1.5) than for individuals receiving the 60 mg/m² dose (23.9%, 1.4). Mean maximum LINE-1 demethylation did not differ between patients who were treatment-naïve (28.6%, 1.6) and patients with relapsed or refractory disease (28.9%, 1.1), nor did mean maximum LINE-1 demethylation differ between patients with (28.1%, 1.4) and without (29.4%, 1.3) objective treatment responses.

The median time to response in patients who achieved a complete response or marrow complete response was 85 days (range 23-512) in the entire study population. The median duration of complete response, partial response, or marrow complete response for the entire population was 203 days, and duration of response was longer for patients in the Compound I-1 60 mg/m² group versus the 90 mg/m² dose (295 vs 207 days).

Among the 58 patients who were dependent on RBC transfusions at baseline, 15 (26%) became RBC transfusion independent for at least 8 weeks and nine (16%) became transfusion independent for at least 16 weeks as shown in TABLE 6. The rates of RBC transfusion independence were similar between Compound I-1 dose levels.

TABLE 6

| | Compound I-1 dose | | | | Disease cohort | | | | | |
|---|---|---|---|---|---|---|---|---|---|---|
| | | | | | | | Relapsed | | | |
| | 60 | | 90 | | Treatment- | | or | | All | |
| | mg/m² | | mg/m² | | naïve | | refractory | | patients | |
| Baseline RBC | 31 | | 27 | | 24 | | 34 | | 58 | |
| dependence | | | | | | | | | | |
| RBC independence | 7 | (23%) | 8 | (30%) | 10 | (42%) | 5 | (15%) | 15 | (26%) |
| for 8 weeks | | | | | | | | | | |
| RBC independence | 5 | (16%) | 4 | (15%) | 6 | (25%) | 3 | (9%) | 9 | (16%) |
| for 16 weeks | | | | | | | | | | |
| Baseline platelet | 13 | | 15 | | 12 | | 16 | | 28 | |
| dependence | | | | | | | | | | |
| Platelet independence | 3 | (23%) | 8 | (53%) | 6 | (50%) | 5 | (31%) | 11 | (39%) |
| for 8 weeks | | | | | | | | | | |
| Platelet independence | 2 | (15%) | 4 | (27%) | 4 | (33%) | 2 | (13%) | 6 | (21%) |
| for 16 weeks | | | | | | | | | | |

Data are n or n (%) of patients with baseline dependence. RBC = red blood cell.

Median overall survival was 460 days (95% CI 384-663) for the entire study population, 611 days (95% CI 408-771) for the Compound I-1 60 mg/m² group, and 399 days (95% CI 303-663) for the 90 mg/m² group, with 67% (95% CI 52-78) 12-month survival and 39% (95% CI 26-52) 24-month survival for the 60 mg/m² group versus 60% (95% CI 45-72) 12-month survival and 30% (95% CI 18-43) 24-month survival for the 90 mg/m² group. Among relapsed/refractory patients, median overall survival was 9.1 months with the 60 mg/m² dose and 12.3 months with the 90 mg/m² dose as shown in FIG. 3. The number of deaths at database lock for the 60 mg/m² group was 18 (67%) of 27 patients and 15 (68%) of 22 in the 90 mg/m² group in the treatment-naïve cohort, and 19 (73%) of 26 for the 60 mg/m² group and 20 (74%) of 27 for the 90 mg/m² group in the relapsed or refractory cohort. No statistically significant difference in overall survival between doses was observed (p=0.47). Median overall survival was 703 days (95% CI 458-920) in the hypomethylating agent treatment-naïve cohort and 352 days (262-505) in the relapsed or refractory disease cohort, with 2-year survival rates of 44% (30-58) in the hypomethylating agent treatment-naïve cohort and 25% (14-38) in the relapsed or refractory disease cohort. Survival within each disease cohort did not differ by Compound I-1 dose (p=0.56 in patients who were treatment-naïve; p=0.89 in patients with relapsed or refractory disease).

Safety Results:

Adverse events occurring in 10% or more of patients are shown in TABLE 7. The incidence of grade 3 or worse adverse events, regardless of relationship to treatment, tended to be lower with the Compound I-I 60 mg/m² dose versus the 90 mg/m² dose (44 [83%] of 53 patients vs 47 [96%] of 49 patients; p=0.054). In the 60 mg/m² group, the most frequent grade 3 or worse adverse events were thrombocytopenia, neutropenia, anemia, febrile neutropenia, and pneumonia. In the 90 mg/m² group, the most frequent grade 3 or worse adverse events were thrombocytopenia, neutropenia, anemia, febrile neutropenia, and pneumonia.

TABLE 7

| Compound I-1 | | | Compound I-1 | | |
|---|---|---|---|---|---|
| 60 mg/m² | | | 90 mg/m² | | |
| (n = 53) | | | (n = 49) | | |
| Grade | Grade | Grade | Grade | Grade | Grade |
| 1-2 | 3 | 4 | 1-2 | 3 | 4 |

Hematological events

| | | | | | | | | | | | | |
|---|---|---|---|---|---|---|---|---|---|---|---|---|
| Anemia | 4 | (8%) | 23 | (43%) | 2 | (4%) | 4 | (8%) | 19 | (39%) | 5 | (10%) |
| Neutropenia | 4 | (8%) | 2 | (4%) | 19 | (36%) | 1 | (2%) | 4 | (8%) | 21 | (43%) |

| | | | | | | | | | | |
|---|---|---|---|---|---|---|---|---|---|---|
| Thrombocytopenia | 1 | (2%) | 0 | 22 | (41%) | 0 | 5 | (10%) | 23 | (47%) |

| | | | | | | | | | | |
|---|---|---|---|---|---|---|---|---|---|---|
| Febrile neutropenia | 0 | 15 | (28%) | 2 | (4%) | 0 | 20 | (41%) | 1 | (2%) |
| Leukopenia | 0 | 2 | (4%) | 5 | (9%) | 0 | 2 | (4%) | 6 | (12%) |

Non-hematological events

| Injection site pain | 19 | (36%) | 0 | | 0 | 21 | (43%) | 0 | | 0 | |
|---|---|---|---|---|---|---|---|---|---|---|---|
| Injection site hematoma | 6 | (11%) | 0 | | 0 | 11 | (22%) | 0 | | 0 | |
| Injection site nodule | 9 | (17%) | 0 | | 0 | 8 | (16%) | 0 | | 0 | |
| Fatigue | 21 | (40%) | 4 | (8%) | 0 | 13 | (27%) | 6 | (12%) | 0 | |
| Diarrhea | 17 | (32%) | 0 | | 0 | 21 | (43%) | 1 | (2%) | 0 | |
| Nausea | 18 | (34%) | 0 | | 0 | 19 | (39%) | 1 | (2%) | 0 | |
| Pneumonia | 2 | (4%) | 13 | (25%) | 0 | 2 | (4%) | 14 | (29%) | 1 | (2%) |

| Constipation | 15 | (28%) | 0 | | 0 | 18 | (37%) | 0 | | 0 |
|---|---|---|---|---|---|---|---|---|---|---|
| Cough | 15 | (28%) | 0 | | 0 | 15 | (31%) | 0 | | 0 |
| Contusion | 16 | (30%) | 0 | | 0 | 11 | (22%) | 1 | (2%) | 0 |
| Decreased appetite | 12 | (23%) | 0 | | 0 | 15 | (31%) | 0 | | 0 |
| Insomnia | 11 | (21%) | 0 | | 0 | 14 | (29%) | 0 | | 0 |
| Dyspnea | 10 | (19%) | 1 | (2%) | 0 | 12 | (24%) | 1 | (2%) | 0 |
| Stomatitis | 3 | (6%) | 2 | (4%) | 0 | 15 | (31%) | 3 | (6%) | 0 |
| Hypokalemia | 7 | (13%) | 1 | (2%) | 0 | 11 | (22%) | 3 | (6%) | 0 |
| Vomiting | 10 | (19%) | 0 | | 0 | 11 | (22%) | 1 | (2%) | 0 |
| Dizziness | 12 | (23%) | 0 | | 0 | 9 | (18%) | 0 | | 0 |
| Hypomagnesaemia | 13 | (25%) | 0 | | 0 | 7 | (14%) | 1 | (2%) | 0 |
| Peripheral edema | 12 | (23%) | 0 | | 0 | 9 | (18%) | 0 | | 0 |
| Epistaxis | 10 | (19%) | 1 | (2%) | 0 | 8 | (16%) | 1 | (2%) | 0 |
| Headache | 6 | (11%) | 1 | (2%) | 0 | 13 | (27%) | 0 | | 0 |
| Rash | 11 | (21%) | 0 | | 0 | 9 | (18%) | 0 | | 0 |
| Asthenia | 9 | (17%) | 1 | (2%) | 0 | 9 | (18%) | 0 | | 0 |
| Pain in extremity | 9 | (17%) | 1 | (2%) | 0 | 8 | (16%) | 0 | | 0 |
| Petechiae | 11 | (21%) | 0 | | 0 | 7 | (14%) | 0 | | 0 |
| Cellulitis | 3 | (6%) | 3 | (6%) | 0 | 4 | (8%) | 7 | (14%) | 0 |
| Pyrexia | 3 | (6%) | 1 | (2%) | 0 | 8 | (16%) | 3 | (6%) | 0 |
| Arthralgia | 7 | (13%) | 2 | (4%) | 0 | 5 | (10%) | 0 | | 0 |
| Back pain | 6 | (11%) | 2 | (4%) | 0 | 5 | (10%) | 1 | (2%) | 0 |
| Dyspepsia | 5 | (9%) | 0 | | 0 | 9 | (18%) | 0 | | 0 |
| Myalgia | 10 | (19%) | 0 | | 0 | 4 | (8%) | 0 | | 0 |
| Upper respiratory tract | 5 | (9%) | 0 | | 0 | 8 | (16%) | 1 | (2%) | 0 |
| infection | | | | | | | | | | |
| Hyponatremia | 3 | (6%) | 4 | (8%) | 0 | 5 | (10%) | 1 | (2%) | 0 |
| Nasal congestion | 6 | (11%) | 0 | | 0 | 7 | (14%) | 0 | | 0 |
| Oropharyngeal pain | 6 | (11%) | 0 | | 0 | 7 | (14%) | 0 | | 0 |
| Hypotension | 4 | (8%) | 1 | (2%) | 0 | 6 | (12%) | 1 | (2%) | 0 |
| Night sweats | 4 | (8%) | 0 | | 0 | 8 | (16%) | 0 | | 0 |
| Dehydration | 3 | (6%) | 1 | (2%) | 0 | 6 | (12%) | 1 | (2%) | 0 |

| Muscle spasms | 8 | (15%) | 0 | 0 | 3 | (6%) | 0 | | 0 |
|---|---|---|---|---|---|---|---|---|---|
| Abdominal pain | 7 | (13%) | 0 | 0 | 2 | (4%) | 1 | (2%) | 0 |

| | | | | | | | | |
|---|---|---|---|---|---|---|---|---|
| Rhinorrhea | 6 | (11%) | 0 | 0 | 4 | (8%) | 0 | 0 |

| | | | | | | | | | | |
|---|---|---|---|---|---|---|---|---|---|---|
| Sepsis | 0 | 2 | (4%) | 1 | (2%) | 0 | 1 | (2%) | 4 | (8%) |

| | | | | | | | | | | |
|---|---|---|---|---|---|---|---|---|---|---|
| Transfusion reaction | 4 | (8%) | 1 | (2%) | 0 | 4 | (8%) | 1 | (2%) | 0 |

| | | | | | | | | |
|---|---|---|---|---|---|---|---|---|
| Weight decreased | 6 | (11%) | 0 | 0 | 4 | (8%) | 0 | 0 |

Data are n (%). Two treatment-related deaths occurred (pneumonia with 90 mg/m² and septic shock with 60 mg/m².) Events occurring in at least 10% of patients and all grade 3 or worse events are shown.

The most common serious adverse events, regardless of relationship to study treatment, were febrile neutropenia (16 [30%] of 53 patients receiving 60 mg/m² and 20 [41%] of 49 patients receiving 90 mg/m²) and pneumonia (11 [21%] patients receiving 60 mg/m² and 14 [29%] receiving 90 mg/m²). 24 (24%) of 102 patients treated had drug-related serious adverse events (11 [21%] of 53 receiving 60 mg/m² and 13 [27%] of 49 receiving 90 mg/m²). Overall, the most common drug-related serious adverse events were febrile neutropenia (11 [11%] of 102 patients), pneumonia (seven [7%]), anemia (three [3%]), and thrombocytopenia (three [3%]).

Seven patients died from serious adverse events, including six patients with relapsed or refractory myelodysplastic syndromes (three each in the Compound I-1 60 and 90 mg/m² groups) and one patient who was hypomethylating agent treatment-naïve with myelodysplastic syndromes (60 mg/m² group). Serious adverse events that led to death were sepsis (two patients with relapsed or refractory disease in the 90 mg/m² group), septic shock (one patient with relapsed or refractory disease in the 60 mg/m² group), pneumonia (one patient with relapsed or refractory disease in each group), respiratory failure (one patient with relapsed or refractory disease in the 60 mg/m² group), and subdural hematoma (one patient who was treatment-naïve in the 60 mg/m² group). Only two serious adverse events that led to death were considered treatment related by the investigator (pneumonia with 90 mg/m² and septic shock with 60 mg/m²).

Overall, 12 (12%) of 102 patients discontinued treatment with Compound I-1 due to adverse events. The overall all-cause mortality with Compound I-1 was low. 30-day all-cause mortality occurred in one (1%) of 102 patients, 60-day all-cause mortality occurred in four (4%) patients, and 90-day all-cause mortality occurred in nine (9%) patients. Most deaths occurred in the 90 mg/m² group.

### Example 2: Subgroup Analysis of Phase 1/2 Trial

Data obtained during the trial of EXAMPLE 1 were further analyzed for selected subgroups of patients including patients administered at least 4 treatment cycles, patients administered at least 6 treatment cycles, patients with less than or at least 5% bone marrow (BM) blasts, patients with transfusion dependence or independence at baseline, patients with baseline ECOG PS of 0-1 or 2, and patients with or without mutations in TP53, TET-2, or DNMT3a.

Number of Treatment Cycles Administered:

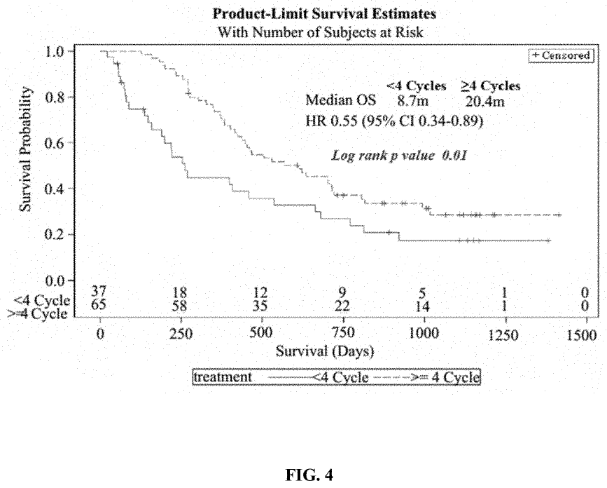

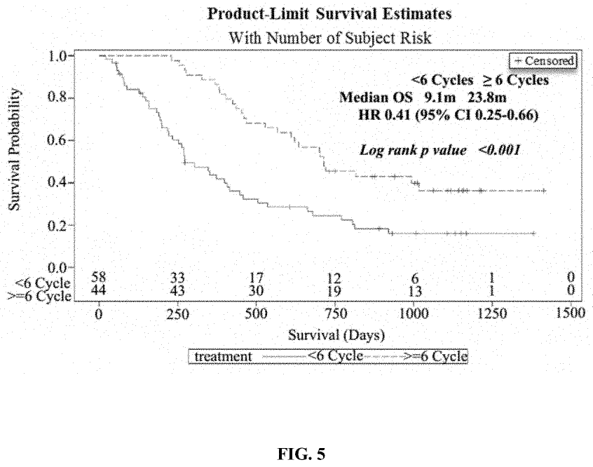

Analysis of data from all 102 patients treated with Compound I-1 showed a median survival of 8.7 months for patients receiving less than 4 treatment cycles and 20.4 months for patients receiving 4 or more treatment cycles, as shown in FIG. 4. The hazard ratio (HR) favored the group receiving at least 4 treatment cycles (HR=0.55; 95% CI 0.34-0.89). Similar results were seen when analyzing patients receiving less than 6 treatment cycles compared to patients receiving 6 or more treatment cycles. The OS was 9.1 months for patients receiving less than 6 treatment cycles and 23.8 months for patients receiving 6 or more treatment cycles, with a HR of 0.41 (95% CI 0.25-0.66) as shown in FIG. 5.

Clinical Prognostic Factors:

Patients were analyzed according to the presence of BM blasts, transfusion dependence, and ECOS PS at baseline. P values were calculated for each comparison based on a log-rank test of the overall survival curves. Results of these analyses are summarized in TABLE 8. Patients with at most 5% BM blasts at baseline exhibited a statistically significant increase in median OS compared to patients with more than 5% BM blasts at baseline. Patients who were transfusion independent at baseline exhibited a statistically significant increase in median OS. Though an increase in median OS was seen in patients with a baseline ECOG PS of 0-1 compared to patients with a baseline ECOG PS of 2, the increase was not statistically significant (p=0.161).

TABLE 8

| | Median OS | P |
|---|---|---|
| | (months) | value* |
| Baseline BM Blasts | 12.3 | 0.005 |
| >5% | 22.7 | |
| ≤5% | | |
| Baseline RBCs | 11.7 | 0.006 |
| Transfusion Dependence | 20.4 | |
| Transfusion Dependent | | |
| Transfusion Independent | | |
| Baseline ECOG PS | 15.6 | 0.161 |
| 0-1 | 8.7 | |
| 2 | | |

### Example 3: Follow-Up Study on Trial from Example 1 for Landmark Analysis

Methods: Landmark response based on 2006 International Working Group (IWG) criteria, and overall survival (OS) analyses for patients alive at or beyond month 3 and month 5 (time of planned start of cycle 4 and cycle 6, respectively) were conducted based upon the study results from the trial of EXAMPLE 2. Objective response (OR) was described as patients who had Complete Response (CR), Partial Response (PR), marrow (m)CR, or Hematological Improvement (HI). Landmark OS was compared between patients who received at least 4 or 6 cycles and those who did not receive at 4 or 6 cycles of treatment. The landmark methodology reduced the bias of early deaths before cycles 4 and 6 and attributed a survival benefit in those patients who did not die early and were able to get more cycles. The results in responding and non-responding patients were also compared to see whether the survival benefit was restricted to responding patients only.

Results:

The study completed enrolment with 102 patients: 53 patients after HMA failure (relapsed/refractory or r/r), and 49 HMA-naïve patients (Treatment Naïve or TN) with a median follow up for the entire study of 3.2 years (IQR (interquartile range; 2.9-3.5 years). Median age was 71 and 72 years for TN MDS/CMML and r/r MDS/CMML patients, respectively. Median OS was 23.4 months (m) for TN MDS/CMML patients and 11.7 m for r/r MDS/CMML patients.

Of the 102 patients treated, 37 patients (36.3%) and 58 (56.9%) received less than 4 and 6 cycles, respectively. The landmark analysis population was 91 patients for the 4-cycle analysis and 87 patients for the 6-cycle analysis. TABLE 9 below details the data set for the landmark analyses.

TABLE 9
Data Set for Landmark Analysis and Treatment
Exposure of Compound I-1 Phase 2

| Data Set for Landmark Analysis | Total MDS/CMML (n = 102) | |
|---|---|---|
| Alive at 3 months¹ | 91 | (89%) |
| Discontinued (<4 cycles)³ | 26 | (29%) |
| Continued (>4 cycles)³ | 65 | (71%) |
| Alive at 5 Months² | 87 | (85%) |
| Discontinued (<6 cycles)⁴ | 43 | (49%) |
| Continued (>6 cycles)⁴ | 44 | (51%) |

¹Data Set for Landmark Analysis at 4 Cycles
²Data Set for Landmark Analysis at 6 Cycles
³% Calculated from the dataset alive at 3 months
⁴% Calculated from dataset alive at 5 months

No major baseline characteristics difference was observed between patients who received at least 4 and 6 cycles and those who did not in the patients included in the landmark analyses. TABLE 10 below details the baseline characteristic of the analyzed patients.

TABLE 10
Baseline Characteristics of Patients in Landmark Analyses

| | Alive at 3 Months | | Alive at 5 Months | |
|---|---|---|---|---|
| | (n = 91) | | (n = 87) | |
| | <4 cycles | >4 cycles | <6 cycles | >6 cycles |
| | (n = 26) | (n = 65) | (n = 43) | (n = 44) |
| Age median | 74 | 71 | 67 | 72 |
| (range) y | (18-85) | (52-86) | (18-83) | (52-82) |
| Sex M/F % | 58%/42% | 71%/29% | 58%/42% | 75%/25% |
| ECOG PS | 73%/27% | 91%/9% | 88%/12% | 89%/11% |
| 0-1/2-3% | | | | |
| BM Blasts >5% | 50% | 49% | 51% | 43% |
| RBCs Transfusion | 62% | 51% | 56% | 48% |
| Dependence | | | | |
| MDS IPSS Int 2/ | 46% | 49% | 49% | 45% |
| High Risk % | | | | |
| CMML | 23% | 23% | 23% | 23% |

In the studied patients, the primary reasons for treatment discontinuation before cycle 4 or 6, respectively, were patient decision (9.8% and 11.8%), and investigator decision (5.9% and 9.8%) while early progression accounted for 3.9% and 10.8% of those patients. TABLE 11 details the primary reasons for treatment discontinuation.

TABLE 11
Primary Reasons for Treatment Discontinuation
in Landmark Analysis Data Set

| | |
|---|---|
| Alive at 3 Months | Alive at 5 Months |
| (n = 91) | (n = 87) |

| | | |
|---|---|---|
| Treatment | <4 cycles n = 26 | <6 cycles n = 43 |
| Discontinuation | | |

Primary Reasons for Treatment Discontinuation N %¹

| | | | | |
|---|---|---|---|---|
| Patient or Investigator | 13 | (50%) | 17 | (39%) |
| Decision | | | | |
| Progressive Disease | 4 | (15%) | 11 | (26%) |
| Adverse Events | 1 | (4%) | 2 | (5%) |

| | | |
|---|---|---|
| Death | 0 | 0 |

| | | | | |
|---|---|---|---|---|
| Other | 8 | (31%) | 13 | (30%) |

¹% calculated for those who had discontinued treatment but alive at 3 and 5 months, respectively. About half of patients alive at 3 months discontinued treatment before 4 cycles because of patient or investigator decision, which was the primary cause of discontinuation in patients alive at 3 and 5 months.

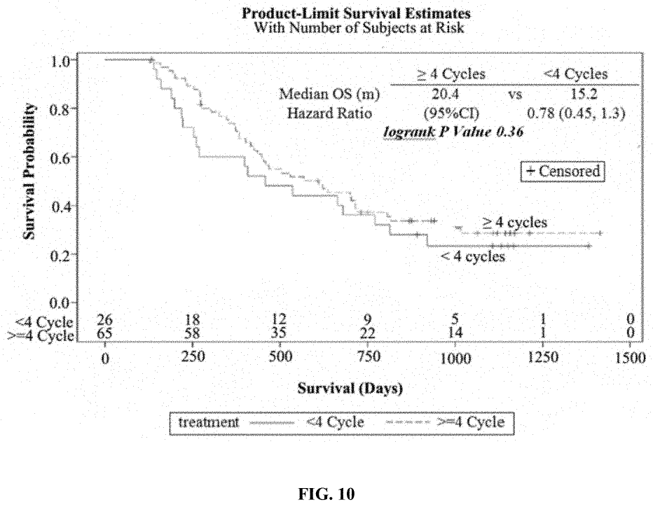

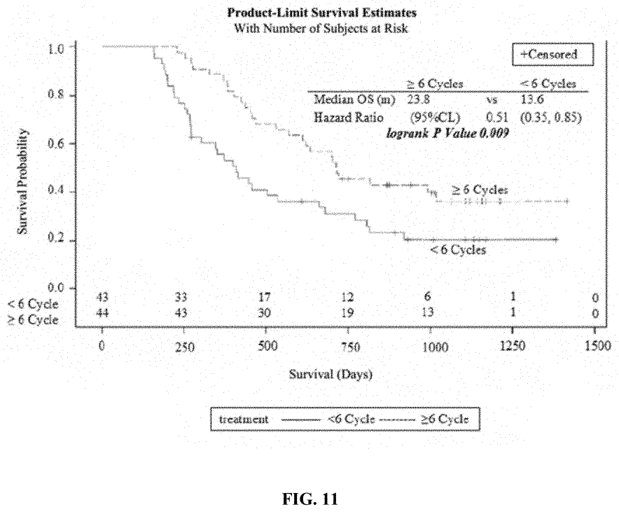

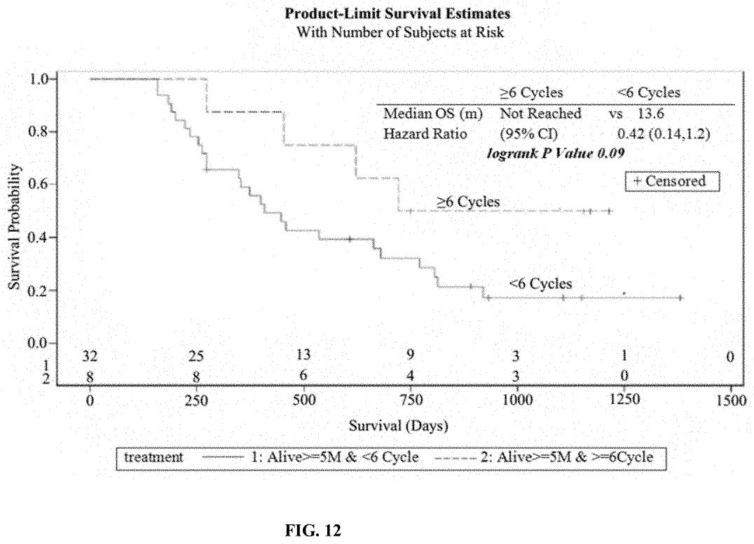

In the landmark analysis, patients who received at least 4 cycles (65 patients) had an OR rate of 68% (44 patients) compared to 15% in 26 patients who received <4 cycles (p <0.0001) and median OS of 20.4 m compared to 15.2 m, respectively (HR 0.78, 95% CI 0.45-1.3, p 0.36; FIG. 10). Those who received at least 6 cycles (44 patients) had an OR rate of 82% compared to 26% in 43 patients who received <6 cycles (p<0.0001), and median OS of 23.8 m vs 13.6 m, respectively (HR 0.51, 95% CI 0.3-0.85, p 0.009; FIG. 11). Results were consistent when r/r MDS/CMML and HMA-naïve MDS/CMML were analyzed separately. Landmark OS analysis also favored those who received Compound I-1 for at least 4 or 6 cycles compared to those who received <4 and <6 cycles even in the absence of objective response (OS HR of 0.82 and 0.42, respectively) but the sample size was small to show statistical significance (p 0.58 and 0.10, respectively). FIG. 12 shows the overall survival by number of cycles in patients who were alive at 5 months with no objective response (n=40).

In this study of 102 MDS/CMML patients treated with the HMA Compound I-1, patients who were alive at the planned start of cycle 4 and cycle 6 did not continue treatment primarily because of patient or investigator decision in addition to early progression. Those patients who were alive and continued treatment for at least 4 or 6 cycles achieved highly significant objective response benefit compared to those who did not received treatment for at least 4 or 6 cycles. Survival benefit was highly significant for those who received at least 6 cycles and was not restricted to patients who had an objective response.

### Example 4: Phase 3 Trial

A Phase 3 randomized trial comparing Compound I-1 to a treatment choice of azacitidine, decitabine, or Low Dose Ara-C(LDAC) in patients with AML was performed.

Study Design and Methods:

The patients were randomized 1:1 to either Compound I-1 (60 mg/m²/d subcutaneous injection for 5 days of a 28-day cycle), or a preselected treatment choice of azacitidine, decitabine, or LDAC at standard doses. AML diagnosis and response status were assessed by an independent blinded central pathologist. CR and OS were the co-primary endpoints.

815 patients were randomized to Compound I-1 (n=408) or a treatment choice (TC) of azacitidine, decitabine, or LDAC (n=407). Preselected treatment choices were decitabine (43%), azacitidine (42%), and LDAC (15%). Baseline variables were well balanced across the 2 arms. Median age was 76 years for both arms, patients ≥75 years were 62% vs 62.4%, ECOG PS 2-3 were reported in 50.5% vs 50.3% (including 10.8% and 8.8% PS 3), poor risk cytogenetics 34.4% vs 34.6%, secondary AML 36.3% vs 36.9%, WBCs ≥20×10⁹/L 15.2% vs 14.3%, median BM blasts 56% vs 53%, and TP53 mutations 12.5% vs 10.6% for Compound I-1 vs treatment choice, respectively.

AML diagnosis was centrally confirmed in 95.1% of patients. Median follow-up was 25.5 months and median number of treatment cycles was 5 for both arms, but 42% of patients only received 1-3 cycles.

The co-primary endpoints analyses showed a CR rate of 19.4% vs 17.4% for Compound I-1 vs treatment choice (p-value of 0.48).

Diagnosis and Eligibility Criteria:

The study group contained patients who were treatment naïve for acute myeloid leukemia (TN-AML), who were not eligible for intensive chemotherapy due to age greater than or equal to 75 years, or possession of at least one of the following characteristics: poor performance status (Eastern Cooperative Oncology Group Performance Status of 3), clinically significant heart or lung comorbidities, liver transaminases over 3 times the upper limit of normal, other contraindications to anthracycline therapy, or other comorbidities incompatible with intensive remission induction chemotherapy.

Subjects who were candidates for intensive remission induction chemotherapy were excluded, as were subjects who were not candidates for any active therapy with TC comparators. Subjects were also excluded if they had extramedullary central nervous system AML; second malignancy requiring active therapy; prior treatment with decitabine or azacitidine; significantly compromised liver function; refractory congestive heart failure unresponsive to medical treatment, active infection resistant to all antibiotics, or advanced pulmonary disease requiring >2 L/min oxygen.

Dosing and Administration of Compound I-1:

Compound I-1 powder was reconstituted at a concentration of 100 mg/mL and administered subcutaneously (SC) at a dose of 60 mg/m² daily on days 1-5 of a 28-day cycle.

Dosing and Administration of TC:

LDAC was given at a dose of 20 mg SC twice daily (BID) on days 1-10 of a 28-day cycle. Decitabine was given at a dose of 20 mg/m² as a 1-hour intravenous (IV) infusion on days 1-5 of a 28-day cycle. Azacitidine was given IV or SC daily on days 1-7 of a 28-day cycle at a dose of 75 mg/m². Other TC treatment parameters such as dose adjustment guidelines followed locally approved prescribing information and institutional standard practice.

Duration of Treatment:

Compound I-1 was given for at least 6 cycles in the absence of unacceptable toxicity or disease progression requiring alternative therapy. Beyond 6 cycles, treatment continued as long as the subject continued to benefit based on investigator judgment.

Duration of treatment for TC followed locally approved prescribing information and institutional standard practice.

Study Endpoints:

Two co-primary endpoints were used: complete response (CR) rate based on modified International Working Group (IWG) 2003 AML Response Criteria and OS (the number of days from randomization to death). Secondary endpoints included CRc (CR+CR with incomplete blood count recovery [CRi]+CR with incomplete platelet recovery [CRp]) rate, number of days alive and out of the hospital (NDAOH), progression-free survival (PFS), health related quality of life (QOL) by EQ-5D (consisting of the EQ-5D-5L descriptive system and the EQ Visual Analogue Scale [EQ VAS]), duration of CR (the time from first CR to time of relapse), incidence and severity of adverse events (AEs), and 30- and 60-day all-cause mortality.

Statistical Methods:

Statistical analysis sets were defined as follows. All subjects analysis set=all screened subjects; efficacy analysis set=all randomized subjects (according to randomization); safety analysis set=all treated subjects (according to treatment received); PK analysis set=all subjects who had PK samples collected and successfully analyzed.

Assuming a CR rate of approximately 0.20 for subjects treated in the TC group (all TC therapies combined) and assuming an increase in CR rate to 0.30 or higher could be achieved by treating subjects with compound I-1, approximately 800 subjects (approximately 400 per treatment group) provided approximately 89% power to detect the overall difference of 0.10 when using a 2-sided Cochran Mantel-Haenszel test having 2-sided alpha level of 0.04. For survival, the primary analysis performed after 670 death events provided 90% power to detect a HR of approximately 0.78 (a difference in median survival of 7 months in the TC group versus 9 months in the compound I-1 group), when using a 2-sided stratified log-rank test at an 0.05 alpha level.

Test Sequence:

By trial design, the overall (2-sided) alpha level of 0.05 was split between the co-primary endpoints of CR (0.04) and OS (0.01). CR rate was tested first in the sequence at an alpha level of 0.04. A positive CR analysis (p≤0.04) served as the gatekeeper to subsequent analyses. If the test for CR was positive, then hierarchical analyses were to be conducted at the 0.05 alpha level for OS, and subsequently for CRc rate, NDAOH, and PFS (in that order) if the preceding test was positive. If the test for CR was not significant, then hierarchical analyses were to be conducted at the 0.01 level for OS, CRc, NDAOH, and PFS.

Co-Primary Endpoints:

CR rate was compared between Compound I-1 and TC groups using Cochran Mantel-Haenszel test at an alpha of 0.04, stratified by the stratification factors used at randomization. OS curves were estimated using Kaplan-Meier (K-M) method and formally compared between Compound I-1 and TC groups using a 2-sided stratified log-rank test, stratified by the same stratification factors used at randomization. Sensitivity analyses were conducted for CR rate and OS to evaluate the robustness of the treatment effect.

Secondary Endpoints:

CRc rate was compared between treatment groups using a Cochran Mantel-Haenszel test with the same stratification variable as for CR and OS. NDAOH was compared between treatment groups using an analysis of variance model (ANOVA) with stratification variables used as fixed factors. PFS was compared between treatment groups using a stratified log-rank test.

The number of RBC and platelet transfusions up to Day 180 were summarized descriptively. 95% CI of the mean was also provided. The EQ-5D-5L index values and EQ VAS up to Day 180 were analyzed descriptively and using a mixed model approach for repeated measures. Duration of CR was estimated using a K-M method for subjects who achieved a CR during the study, and a separate K-M analysis including all subjects was conducted (using a 0-day event duration for subjects who did not achieve CR).

The incidence and severity of adverse events (AEs) and 30- and 60-day all-cause mortality were summarized with descriptive statistics. No formal statistical testing between the treatment groups was planned for safety assessments.

Exploratory Endpoints:

Subgroup analyses were performed to explore the influence of baseline variables and the individual TC therapy administered on the efficacy outcomes of CR rate and OS.

A previously developed Population PK model was applied to calculate model-derived estimates for total cycle exposures (Cₘₐₓ and AUC) from sparse PK samples collected in Cycle 1 for Compound I-1 and decitabine for use in exposure-response analysis for efficacy and safety. The analyses of exposure-efficacy and exposure-safety relationships were performed separately for patients who received Compound I-1 and patients who received decitabine IV. The following analyses were performed to assess relationships of CR with exposure for each exposure measure: frequencies of CR were tabulated and compared between tertiles of exposure; distributions of exposure were compared for patients with and without CR using box plots; and probability of CR was explored using logistic regression models.

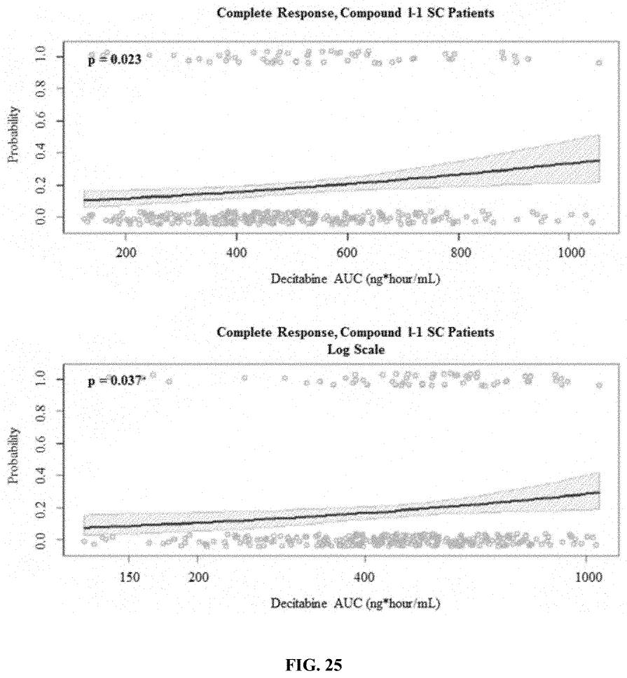

FIG. 25 shows the logistic regression for probability of complete response versus the decitabine adjusted area under the curve (Compound I-1 arm). The circles show the observed response (0=no response; 1=response; vertically jittered for better visualization). The lines show the logistic regression lines. The shaded regions are the 90% confidence intervals for the regression line.

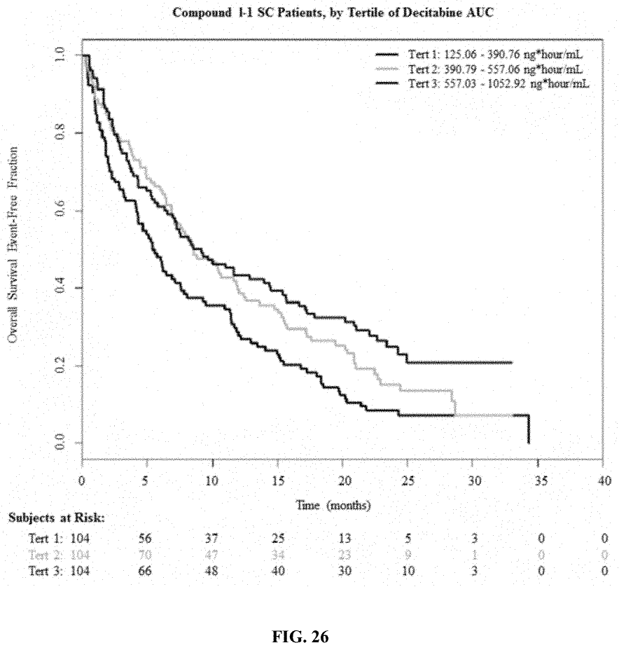

FIG. 26 is a Kaplan-Meier plot for time to overall survival by the tertiles of decitabine adjusted area under the curve (Compound I-1 arm).

Subject Disposition:

815 subjects were randomized in the study (n=408 for Compound I-1; n=407 for TC) and 793 subjects were treated (n=401 for Compound I-1; n=392 for TC). Preselected TC prior to randomization was decitabine, 351 subjects (43%); azacytidine, 340 subjects (42%); and LDAC, 124 subjects (15%). Overall, the median follow-up time was 766 days (IQR 671-896 days). Reasons for treatment discontinuation and study withdrawal were similar in the Compound I-1 and TC groups. Overall, the most common reasons for treatment discontinuation were progressive disease (34.8%), death (29.0%), subject decision (10.7%), and adverse event (9.7%). The most common reason why subjects withdrew from the study was death (81.2%). As of the data cut-off date, 51 subjects (6.3%) continued to receive study treatment, more on Compound I-1 (34 subjects, 8.3%) than TC (17 subjects, 4.2%).

Subject Demographics and Baseline Characteristics:

The overall mean age of subjects was 75.9 years (range: 56 to 94 years). Most subjects were white (73.9%) and 58% were male. Most subjects (62.2%) were ≥75 years old and approximately half of subjects had ECOG PS 2 or 3 (50.4%), including 9.8% with ECOG PS 3 (10.8% on Compound I-1, 8.8% on TC). In addition, 34.5% had poor risk cytogenetics, and 36.6% of subjects had secondary AML. The median percent blasts as determined by central pathologist was 15.0% in peripheral blood (PB) and 56.0% in bone marrow (BM). 14.7% of subjects had a white blood cell (WBC) count of ≥20,000/μL and 69.7% of subjects had >30% BM blasts. Baseline demographics, disease characteristics, and hematologic parameters were well balanced between Compound I-1 and TC groups.

Efficacy Results:

In accordance with the previously-defined hierarchical testing order, CR was tested first. The test for CR rate was not significant at the 0.04 level; therefore, the test for OS was conducted at an alpha level of 0.01. The test for OS was not significant and per the pre-defined hierarchical testing plan, no further testing was performed.

Primary Endpoints:

The CR rate was 19.4% for Compound I-1 and 17.4% for TC, the difference was 1.92% higher for Compound I-1 (96% CI: −3.67-7.5); and was not statistically significant (p=0.4820; stratified CMH test). Overall, the results of the planned sensitivity analyses for CR rate were consistent with the results of the primary analysis. The CR rates were numerically higher for Compound I-1 than for TC for each of the sensitivity analyses conducted (i.e., unstratified Chi-square, all treated subjects, excluding not evaluable (NE) subjects, and excluding unconfirmed AML); and the odds ratio (OR) of the Compound I-1 vs TC logistic regression was >1 (OR 1.14; 95% CI 0.80-1.63).

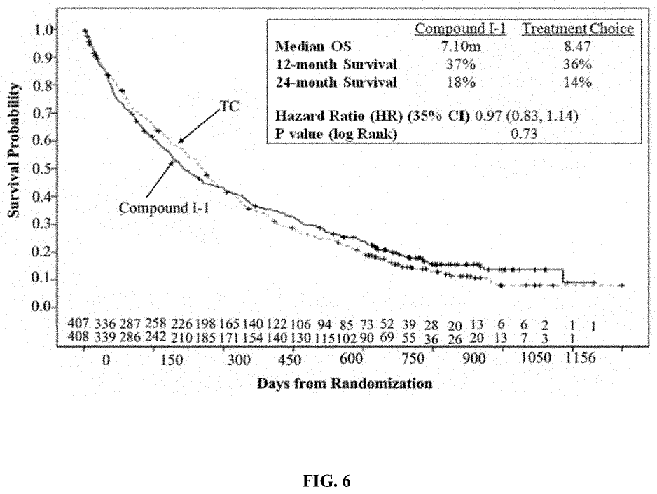

As depicted in FIG. 6, median OS was 213 days (7.1 months) for Compound I-1 and 254 days (8.47 months) for TC. The stratified log-rank test did not reach statistical significance (p=0.7328). The HR was 0.97 (95% CI 0.83-1.14). Survival was shorter for Compound I-1 than for TC in the 25^{th} and 50^{th} percentiles. The K-M survival curves intersect at approximately 300 days (10 months) and survival was longer for Compound I-1 than for TC for the 75^{th} percentile (19.5 vs 16.8 months). The 12-month survival rates were similar between Compound I-1 and TC (37% and 36%); and the 24-month survival rate was numerically higher for Compound I-1 than for TC (18% vs 14%). The overall survival hazard ratio favored Compound I-1 vs each treatment choice (hazard ratio of less than 1). Overall, the results of the planned sensitivity analyses for OS (i.e., as treated safety analysis set, additional censoring for antileukemia treatment, and excluding subjects with unconfirmed AML) were consistent with the results of the primary analysis. In a post hoc exploratory survival analysis for subjects who achieved any CR (CR, CRp, or CRi) the point estimate of the HR favored Compound I-1 compared to TC (HR: 0.72, 95% CI 0.50-1.05).

Secondary Endpoints:

CRc rates were similar between the Compound I-1 and TC groups (22.8% and 22.4%, respectively). The mean number of days alive and out of the hospital (NDAOH) over 6 months (3.3 and 3.5 months) and per patient-year rates (297 and 299 days) were similar between the Compound I-1 and TC groups. PFS K-M survival curves were similar between the Compound I-1 and TC groups (HR: 0.99, 95% CI 0.861.15). Median PFS was 5.3 months and 5.5 months for Compound I-1 and TC, respectively. Transfusions were similar between Compound I-1 and TC during the first 6 months (means: 16.2 and 15.6 units for RBC; 12.5 and 14.4 units for platelets for Compound I-1 and TC, respectively). Over the first 6 months, index EQ-5D-5L scores were similar between Compound I-1 and TC groups (LS mean difference of −0.019, 95% CI −0.058-0.020). Descriptive EQ-5D-5L scores favored TC in the first 6 months. Over the first 6 months, VAS scores slightly improved (increased) from baseline for both groups with a least squares (LS) mean change from baseline of 0.87 for Compound I-1 and 1.13 for TC. The median duration of CR among complete responders was similar between Compound I-1 and TC (7.2 and 7.7 months, respectively).

A summary of trial results pertaining to primary and second endpoints can be seen below in TABLE 12.

TABLE 12

| | | |
|---|---|---|
| Compound I-1 | TC | Treatment |
| (N = 408) | (N = 407) | Difference |

Primary Endpoints

| | | | | | |
|---|---|---|---|---|---|
| Complete Response (CR) | 79 | (19.4%) | 71 | (17.4%) | 1.92 |
| Rate | | | | | |
| 96% Confidence Interval | | | | | (−3.67, 7.50) |
| (CI) | | | | | |
| P value^{a} | | | | | 0.4820 |

Overall Survival

| | | | | |
|---|---|---|---|---|
| Death Events | 336 | (82.4%) | 340 | (83.5%) |

K-M Estimate, days (95% CI)

| | | | | | |
|---|---|---|---|---|---|
| 25th percentile | 71.0 | (59.0, 96.0) | 92.0 | (73.0, 112.0) | |
| Median | 213.0 | (187.0, 255.0) | 254.0 | (223.0, 282.0) | |
| 75^{th} percentile | 584.0 | (498.0, 643.0) | 503.0 | (426, 587) | |
| 12-month survival rate | 0.37 | (0.32, 0.42) | 0.36 | (0.31, 0.40) | |
| (95% CI) | | | | | |
| 24-month survival rate | 0.18 | (0.14, 0.22) | 0.14 | (0.11, 0.18) | |
| (95% CI) | | | | | |
| Primary Stratified Log-Rank | | | | | 0.7328 |
| Test^{b} | | | | | |
| Cox Regression HR | | | | | 0.97 (0.83, 1.14) |
| (95% CI)^{c} | | | | | |

Secondary Endpoints

| CRc (CR + CRi + CRp) Rate | 93 | (22.8%) | 91 | (22.4%) | 0.39 |
|---|---|---|---|---|---|
| 95% CI | | | | | (−5.34, 6.13) |
| NDAOH in First 6 Months | | | | | |
| Mean (SD) | 98.1 | (63.60) | 105.7 | (63.58) | |
| LS Mean | 98.9 | | 106.7 | | −7.8 |
| (95% CI) | (92.4, 105.4) | | (100.2, 113.2) | | (−16.2, 0.7) |
| Progression Free Survival | | | | | |
| (PFS) | | | | | |
| Events | 385 | (94.4%) | 378 | (92.9%) | |
| K-M Estimate, days | | | | | |
| (95% CI) | | | | | |
| Median | 159 | (136.0, 178.0) | 166 | (148.0, 179.0) | |

^{a}Cochran Mantel-Haenszel (CMH) method adjusting for stratification factors used at randomization.
^{b}Log-rank test, stratified by stratification factors used at randomization.
^{c}Cox proportional-hazard model (with treatment group as the independent variable, stratified by the stratification factors used for randomization).

Pharmacokinetics and Exposure Response:

The mean plasma Compound I-1 concentration was highest at the first collection time point of 1.5 hours after injection and declined afterward. Decitabine formed metabolically from Compound I-1 SC administration stayed longer in blood circulation than decitabine following decitabine IV administration. The result confirmed longer exposure to the active metabolite decitabine following Compound I-1 administration.

Among investigated exposure measures, safety (Grade ≥3 AEs) and efficacy outcomes (CR and OS) were most correlated with AUC of active metabolite decitabine exposure after SC Compound I-1 administration.

Safety Results:

The overall extent of exposure was similar for Compound I-1 and TC, with both groups receiving a median of 5.0 cycles (range 1-38 cycles for Compound I-1, 1-34 cycles for TC). Exposure was also similar within the preselected decitabine and azacitidine groups, but for the preselected LDAC group, exposure was longer for Compound I-1 subjects (median 5.0 cycles) than LDAC subjects (median 2.0 cycles).

Adverse Events:

In general, the differences in AE incidence between Compound I-1 and TC regardless of causality were small, but the incidence was numerically higher in the Compound I-1 group than in the TC group for most AE categories except for deaths due to AEs. An overview of AEs between Compound I-1 and TC groups is shown below in TABLE 13.

TABLE 13
Overview of AEs

| | Number (%) of Subjects | | | |
|---|---|---|---|---|
| | Compound I-1 | | TC | |
| | (N = 401) | | (N = 392) | |
| Subjects with any AE | 393 | (98.0%) | 387 | (98.7%) |
| Subjects with any Grade ≥3 AE | 367 | (91.5%) | 343 | (87.5%) |
| Subjects with an AE Leading to | 41 | (10.2%) | 26 | (6.6%) |
| Discontinuation of Study Treatment | | | | |
| Subjects with any Serious AE (SAE) | 325 | (81.0%) | 296 | (75.5%) |
| Deaths (due to an AE) | 115 | (28.7%) | 117 | (29.8%) |
| Other Subjects with an SAE | 210 | (52.4%) | 179 | (45.7%) |
| Subjects with any Related AE | 263 | (65.6%) | 245 | (62.5%) |
| Subjects with any Related Grade ≥3 | 201 | (50.1%) | 169 | (43.1%) |
| AE | | | | |
| Subjects with a Related AE Leading | 15 | (3.7%) | 7 | (1.8%) |
| to Discontinuation of Study | | | | |
| Treatment | | | | |
| Subjects with any Related SAE | 123 | (30.7%) | 85 | (21.7%) |
| Deaths (due to a Related AE) | 17 | (4.2%) | 14 | (3.6%) |
| Other Subjects with a Related SAE | 106 | (26.4%) | 71 | (18.1%) |

The AEs that occurred with highest incidence in Compound I-I subjects were pneumonia, febrile neutropenia, thrombocytopenia, constipation, diarrhea, and neutropenia. In TC subjects, the highest incidence AEs were pyrexia, constipation, nausea, febrile neutropenia, and thrombocytopenia. AEs that occurred at a higher rate in Compound I-I subjects included febrile neutropenia, diarrhea, injection site events, and pneumonia. AEs that occurred at a higher rate in TC subjects included pyrexia, nausea, and vomiting.

Grade ≥3 AEs that occurred with the highest incidence in Compound I-I subjects were febrile neutropenia, pneumonia, thrombocytopenia, neutropenia, anemia, and sepsis. In subjects who received TC, the highest incidence Grade ≥3 AEs were febrile neutropenia, thrombocytopenia, neutropenia, pneumonia, anemia, and sepsis. There was a higher incidence of Grade ≥3 febrile neutropenia and pneumonia in Compound I-I subjects than TC subjects. Compound I-I subjects also had a higher incidence of Grade ≥3 blood and lymphatic system.

The most common related AEs and Grade ≥3 related AEs mirrored the AEs regardless of causality but with a lower incidence. A summary of AEs occurring in TC and Compound I-I groups is shown below in TABLE 14 and TABLE 15.

TABLE 14
Incidence of AEs (All Grades) in >15% of
Subjects in Any Group by Decreasing Incidence

| | Number (%) of Subjects | | |
|---|---|---|---|
| | | Compound I-1 | TC |
| MedDRA Preferred Term | | (N = 401) | (N = 392) |

| | | | | |
|---|---|---|---|---|
| Pneumonia | 144 | (35.9%) | 92 | (23.5%) |
| Febrile Neutropenia | 141 | (35.2%) | 107 | (27.3%) |
| Thrombocytopenia | 127 | (31.7%) | 105 | (26.8%) |
| Constipation | 124 | (30.9%) | 114 | (29.1%) |
| Diarrhea | 124 | (30.9%) | 88 | (22.4%) |
| Neutropenia | 115 | (28.7%) | 89 | (22.7%) |
| Anemia | 98 | (24.4%) | 87 | (22.2%) |
| Injection Site Events^{a} | 80 | (20.0%) | 49 | (12.5%) |
| Hypokalemia | 95 | (23.7%) | 79 | (20.2%) |
| Pyrexia | 95 | (23.7%) | 117 | (29.8%) |
| Edema Peripheral | 93 | (23.2%) | 78 | (19.9%) |
| Decreased Appetite | 91 | (22.7%) | 62 | (15.8%) |
| Nausea | 91 | (22.7%) | 108 | (27.6%) |
| Cough | 79 | (19.7%) | 66 | (16.8%) |
| Dyspnea | 65 | (16.2%) | 53 | (13.5%) |
| Asthenia | 64 | (16.0%) | 58 | (14.8%) |
| Fatigue | 64 | (16.0%) | 50 | (12.8%) |
| Sepsis | 62 | (15.5%) | 48 | (12.2%) |
| Vomiting | 57 | (14.2%) | 67 | (17.1%) |

^{a}Injection Site Events is a group term with events determined by medical assessment.

TABLE 15
Incidence of Grade ≥3 AEs in ≥5% of subjects in any
Group by Decreasing Incidence (Safety Population)

| | | |
|---|---|---|
| | Compound I-1 | TC |
| MedDRA Preferred Term | (N = 401) | (N = 392) |

| | | | | |
|---|---|---|---|---|
| Febrile Neutropenia | 136 | (33.9%) | 104 | (26.5%) |
| Pneumonia | 118 | (29.4%) | 77 | (19.6%) |
| Thrombocytopenia | 114 | (28.4%) | 92 | (23.5%) |
| Neutropenia | 110 | (27.4%) | 81 | (20.7%) |
| Anemia | 81 | (20.2%) | 70 | (17.9%) |
| Sepsis | 61 | (15.2%) | 47 | (12.0%) |
| Hypokalemia | 33 | (8.2%) | 35 | (8.9%) |
| Leukopenia | 32 | (8.0%) | 28 | (7.1%) |

Serious AEs:

Serious adverse events (SAEs) that occurred with highest incidence were pneumonia (28.4%), febrile neutropenia (25.2%), and sepsis (15.0%) in subjects who received Compound I-1, and febrile neutropenia (22.4%), pneumonia (19.6%), and sepsis (11.2%) in subjects who received TC. While the incidence of these SAEs was slightly higher with Compound I-1, no common SAE in the Compound I-1 group was observed with an incidence that was ≥1.5-fold higher than in the TC group.

Related SAEs mirrored the SAEs regardless of causality but with lower incidence. Related SAEs with highest incidence were febrile neutropenia (13.7%), pneumonia (8.5%), sepsis (4.0%), thrombocytopenia (1.7%), septic shock (1.2%), and anemia (1.0%) in subjects who received Compound I-1, and febrile neutropenia (8.7%), pneumonia (4.8%), sepsis (2.3%), acute kidney injury (1.0%), diarrhea (1.0%), and septic shock (1.0%) in subjects who received TC.

Other Significant AEs:

Fifteen Compound I-1 subjects (3.7%) and 7 TC subjects (1.8%) discontinued treatment due to a related AE; just under half of these discontinuations (7 Compound I-1, 3 TC) occurred due to an infection event. The only related AEs leading to treatment discontinuation in more than 1 subject in either group were septic shock (2 Compound I-1 subjects, 0.5%) and general physical health deterioration (2 Compound I-1 subjects, 0.5%; 1 TC subject, 0.3%).

Treatment delays due to adverse events occurred in under half the subjects (48.1% Compound I-1, 41.8% TC). Dose reductions due to AEs were much less common (6.0% Compound I-1, 3.3% TC). Neutropenia was the most common AE leading to treatment delay and dose reduction.

Other Safety Results:

No medically-important or treatment-related trends were observed in clinical laboratory parameters or other observations related to safety that were not associated with myelosuppression and complications of myelosuppression. No effects on ECG parameters were observed for Compound I-1 or TC.

Analysis of ECG data collected on-drug during the time window to represent the approximate Tₘₐₓ of Compound I-1 and active metabolite decitabine showed no effect on heart rate for Compound I-1. No signal of any effect on atrioventricular conduction or cardiac depolarization was present as measured by the PR and QRS interval durations for any of the treatment groups. No significant effect on cardiac repolarization was observed as measured by the change from baseline to therapy on Compound I-1 or the other treatment groups and the concentration effect modeling also did not show any effect on cardiac repolarization despite the confidence intervals being quite wide. In conclusion, this trial did not demonstrate any effect of Compound I-1 on ECG parameters.

Deaths:

676 deaths occurred during the study and the primary causes of death were similar for Compound I-land TC. Thirty-day, all-cause mortality was similar in subjects who received Compound I-1 (11.2%) and subjects who received TC (9.7%). 60-day mortality was marginally higher for Compound I-1 than TC (20.9% vs 17.1%).

The incidence of AEs with an outcome of death was similar for Compound I-1 (28.7%) and TC (29.8%). AEs with an outcome of death with the highest incidence were sepsis (6.5%), pneumonia (5.0%), and septic shock (2.0%) in subjects who received Compound I-1, and pneumonia (5.6%), sepsis (5.1%), and cardiac arrest (2.0%) in subjects who received TC. TC subjects had a higher incidence of febrile neutropenia with an outcome of death (1.3%) than Compound I-1 subjects (0.2%).

In subjects who received Compound I-1, related AEs with an outcome of death included pneumonia (4 subjects, 1.0%), sepsis (4 subjects, 1.0%), septic shock (3 subjects, 0.7%), and febrile neutropenia, hematophagy histiocytosis, small intestinal hemorrhage, general physical health deterioration, mucormycosis, and osteonecrosis (in 1 subject [0.2%] each). In subjects who received TC, related AEs with an outcome of death included sepsis (4 subjects, 1.0%), pneumonia (3 subjects, 0.8%), and febrile neutropenia, cardiac arrest, septic shock, device related infection, traumatic lung injury, acute respiratory failure and respiratory distress (in 1 subject [0.3%] each).

Analyses of prospectively defined clinical and major molecular genetics variables did not show significant differences of primary outcomes between Compound I-1 and treatment choice in any subgroup except for TP53 mutation status. Patients with baseline TP53 mutations (94 patients) had worse outcomes on Compound I-1 vs treatment choice (overall survival hazard ratio of 1.8 with a 95% CI of 1.17-2.78). Patients without TP53 mutations (696 patients) had a more favorable outcome on Compound I-1 vs treatment choice (overall survival hazard ratio of 0.86 with a 95% CI of 0.73-1.01).

### Example 5: Subgroup Analysis of Phase 3 Trial for Treatment Cycle and Genetic Mutations

Data obtained during the trial of EXAMPLE 4 were further analyzed for selected subgroups of patients including patients who achieved CR, patients who received more than 3 treatment cycles, and patients with or without genetic mutations in FLT-ITD, NPM1, CEBPA, or TP53.

Patients Who Received More than 3 Treatment Cycles:

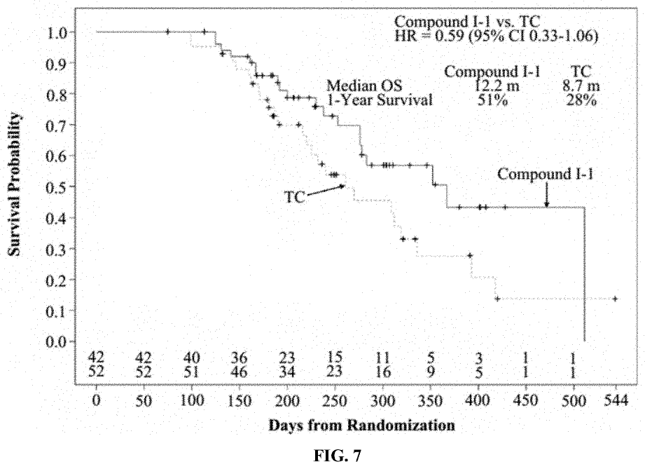

Landmark survival analysis showed that patients who received greater than 3 treatment cycles (i.e. 4 or more cycles) had favorable overall survival on Compound I-1 vs TC (overall survival hazard ratio of 0.78). As shown in FIG. 7, the overall survival for patients who received at least 4 cycles of Compound I-1 treatment had a 51% one-year survival rate compared to those patients who received TC (28% one-year survival). The Compound I-1 treatment was given approximately once every month (e.g., 28 days) for at least 4 cycles. The landmark time for the landmark survival analyses was approximately 4 months. Of the patients that exhibited a higher overall survival rate, about 8-10 patients had TP53 baseline mutations.

Patients Who Achieved CR:

Exploratory analysis showed subjects who achieved any CR (CR (complete response), CRp (complete response with incomplete platelet recovery), or CRi (complete response with incomplete hematologic recovery) lived longer on Compound I-1 compared to TC (HR 0.72; 95% CI 0.50-1.05).

### Example 6: Subgroup Analysis of Phase 3 Trial for Patients Receiving at Least 4 or 6 Cycles of Treatment

Data obtained during the trial of EXAMPLE 4 were further analyzed for selected subgroups of patients including patients who achieved CR in patients who received at least 4 or 6 treatment cycles.

Methods:

TN-AML patients ineligible for intensive chemotherapy (IC) due to age ≥75 y, coexisting morbidities, or ECOG PS 2-3 were randomized 1:1 to either Compound I-1 (60 mg/m²/d SC for 5-days Q28 days) or a preselected treatment choice (TC) of azacitidine (AZA), decitabine (DEC), or low-dose Ara-C(LDAC) at the prescribed dosing schedule. AML diagnosis and response status were assessed by an independent central pathologist blinded to randomization assignment. Complete response (CR) and overall survival (OS) were the co-primary endpoints. The patients' characteristics, number of treatment cycles, reasons for treatment discontinuation, CR, and OS including analyses by number of cycles received including prospective subgroups, and OS analyses of responders and non-responders were analyzed.

815 patients were randomized to Compound I-1 (n=408) or TC (n=407) as described above. Preselected TCs prior to randomization were DEC (n=351; 43%), AZA (n=340; 42%), and LDAC (n=124; 15%). Within the DEC group, 173 patients were given decitabine and 178 patients were given Compound I-1. Within the AZA group, 178 patients were given azacitidine and 162 patients were given Compound I-1. Within the LDAC group, 56 patients were given LDAC and 68 patients were given Compound I-1.

Baseline variables were well balanced across the 2 treatment arms. For Compound I-1 vs TC respectively, age ≥75 y was 62% vs 62.4%, PS 2-3 was 50.5% vs 50.4% (including 10.8% vs 8.8% PS 3), and poor risk cytogenetics was 34.3% vs 34.6%. TABLES 16 and 17 provide the details of the baseline patient characteristics for each treatment group.

TABLE 16
Baseline Characteristics of Patients Receiving >4 Cycles

| | Compound I-1 | TC |
|---|---|---|
| | (n = 235) | n = 241 |
| Median Age (range) years | 76 (57-93) | 76 (59-89) |
| Age >75 yrs | 63% | 59% |
| ECOG Poor PS 2-3 | 43% | 47% |
| Secondary AML | 35% | 40% |
| Poor Risk Cytogenetics | 32% | 34% |
| Bone Marrow Blasts >30% | 67% | 61% |
| Total WBCs >20,000/μL | 12% | 9% |

TABLE 17
Baseline Characteristics of Patients Receiving >6 Cycles

| | Compound I-1 | TC |
|---|---|---|
| | (n = 235) | n = 241 |
| Median Age (range) years | 76 (60-93) | 76 (59-89) |
| Age >75 yrs | 63% | 59% |
| ECOG Poor PS 2-3 | 41% | 47% |
| Secondary AML | 34% | 37% |
| Poor Risk Cytogenetics | 31% | 35% |
| Bone Marrow Blasts >30% | 66% | 60% |
| Total WBCs >20,000/μL | 12% | 9% |

Most patients were assigned to an hypomethylating agent (HMA) at randomization (n=759, 93%) with only 56 patients (7%) randomized to receive LDAC. Both CR (19.4% for Compound I-1 and 17.4% for TC), and OS Hazard Ratio (0.97; 95% CI 0.83-1.14) were similar and not significantly different between Compound I-1 and TC. Many patients in both arms did not receive the recommended minimum of 4 cycles (42.4% vs 40.8% for Compound I-1 vs TC respectively), or 6 cycles (54.2% vs 53.8% for Compound I-1 vs TC). The proportions were well balanced between the 2 treatment arms. Characteristics of patients who received at least 4 or 6 cycles were also well balanced between the 2 treatment arms for age, PS 2-3, secondary AML, poor risk cytogenetics, BM blasts >30%, and proliferative AML (total white cell count ≥20,000/uL). The primary reasons and proportions for treatment discontinuation were similar for the 2 treatments arms. For patients with <4 and <6 cycles, respectively, the reasons were, in descending order, early deaths (16.7% and 20.7% of the overall intention-to-treat (ITT) population), progression (7.6% and 11.7%), adverse events (5.8% and 6.9%), and patient decision (5.5% and 7.1%).

TABLES 18 and 19 provide the details for the discontinuation reasons between the two treatment groups.

TABLE 18
Primary Reasons for Treatment Discontinuation Before 4 Cycles

| | Compound I-1 | TC |
|---|---|---|
| | (n = 408) | (n = 407) |
| Randomized but not Treated | 1.7% | 3.7% |
| Adverse Event | 6.4% | 5.2% |
| Death | 17.6% | 15.7% |
| Progressive Disease | 7.6% | 7.6% |
| Alternative Anti-Leukemia Therapy | 0.7% | 0.5% |
| Patient Decision to Permanently Stop | 5.7% | 5.4% |
| Treatment | | |
| Lost to Follow-up | 0.2% | 0 |
| Other | 2.5% | 2.7% |
| Total % of Patients with <4 cycles | 42.4% | 40.8% |

TABLE 19
Primary Reasons for Treatment Discontinuation Before 6 Cycles

| | Compound I-1 | TC |
|---|---|---|
| | (n = 408) | (n = 407) |
| Randomized but not Treated | 1.7% | 3.7% |
| Adverse Event | 7.4% | 6.4% |
| Death | 22.5% | 18.9% |
| Progressive Disease | 10.8% | 12% |
| Alternative Anti-Leukemia Therapy | 0.7% | 1.2% |
| Patient Decision to Permanently Stop | 6.6% | 7.6% |
| Treatment | | |
| Lost to Follow-up | 0.5% | 0 |
| Other | 3.9% | 3.9% |
| Total % of Patients with <4 cycles | 54.2% | 53.8% |

Results:

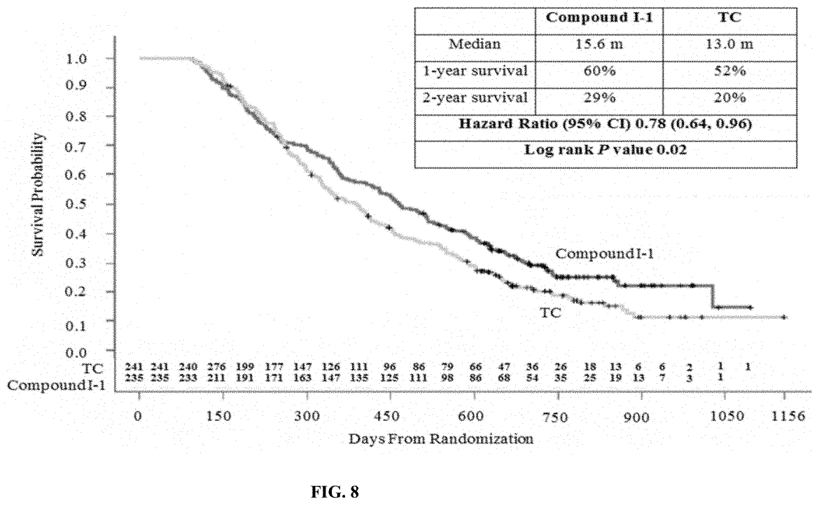

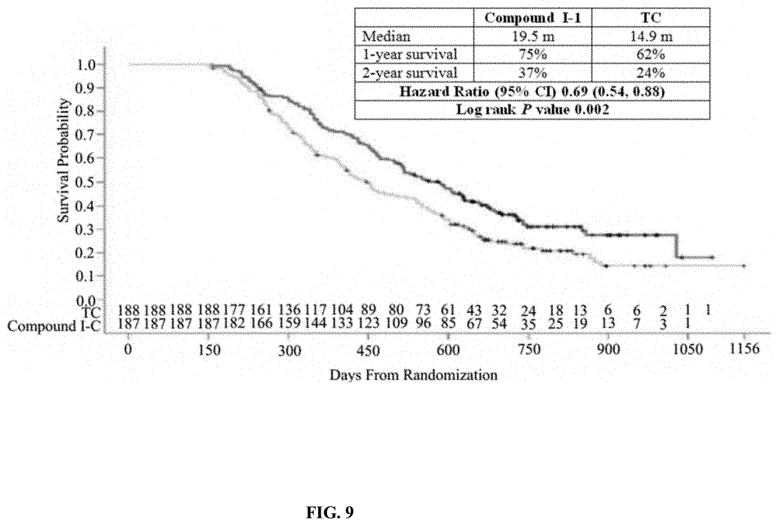

In patients who received at least 4 cycles, more patients achieved CR on Compound I-1 (33.6%) vs TC (28.6%), and median OS was longer on Compound I-1 (15.6 months for Compound I-1 vs 13 for TC, hazard ratio (HR) 0.78, 95% CI 0.64-0.96, log-rank p 0.02, FIG. 8). Similarly, in patients who received at least 6 cycles, there were more CR on Compound I-1 (40.1%) vs TC (36.2%) and median OS was longer on Compound I-1 (19.5 months for Compound I-1 vs 15 for TC, HR 0.69, 95% CI 0.54-0.88, log-rank p 0.002, FIG. 9).

Subgroup analyses of OS in patients who received at least 4 or 6 cycles showed that survival benefit from Compound I-1 over TC was consistent in all prospective subgroups including against each of the 3 TCs (AZA, DEC, and LDAC). OS analyses in patients who received at least 4 or 6 cycles also favored Compound I-1 vs TC in both responders (CR, CRp, CRi, or PR) and non-responders with maximum benefit in patients who received at least 6 cycles (Compound I-1 vs TC OS HR 0.66, 95% CI 0.45-0.96, log-rank p 0.028 for responders, and HR of 0.73, 95% CI 0.53-1.00, log-rank p 0.048 for non-responders).

The results are summarized in TABLE 20 below.

TABLE 20
CR and OS in Patients who Received >4 or >6 Cycles

| | Patients with >4 Cycles | | Patients with >6 Cycles | |
|---|---|---|---|---|
| | Compound | | Compound | |
| | I-1 | TC | I-1 | TC |
| | (n = 235) | (n = 241) | (n = 187) | (n = 188) |
| CR Rate | 33.6% | 28.6% | 40.1% | 36.2% |
| Median | 15.6 | 13 | 19.5 | 15 |
| OS Months | | | | |

| | | |
|---|---|---|
| OS HR | 0.78 (0.64, 0.96), p 0.02 | 0.69 (0.54, 0.88), p 0.002 |
| (95% CI), | | |
| log rank | | |
| p value | | |

### Example 7: Event-Free Survival Analysis of Phase 3 Trial

Data obtained during the trial of EXAMPLE 4 were further analyzed for progression-free survival (PFS) and event-free survival (EFS) in the overall ITT population based on the number of cycles administered. PFS was described as from date of randomization to disease progression or death described as the earliest occurrence of relapse in a responding patient; clinically significant increase in blasts % that necessitated alternative therapy; investigator determined progression, or death. EFS is described as from date of randomization to the earliest occurrence of treatment discontinuation, start of alternative therapy, or death.

As described above, TN-AML ineligible for IC due to age ≥75 y, or comorbidities, or ECOG PS 2-3 were randomized 1:1 to either Compound I-1 (60 mg/m2/d SC for 5-days Q28 days) or a preselected TC of AZA, DEC, or LDAC at their standard regimens. AML diagnosis, and response status by IWG 2003 criteria, were assessed by an independent central pathologist blinded to randomization assignment. CR and OS were co-primary endpoints. PFS was a secondary endpoint calculated from date of randomization to the earliest date of progression by investigators or central assessment, relapse after response, start of an alternative therapy, or death. An EFS analysis was conducted post hoc using the concept of time to treatment failure. EFS was therefore calculated from date of randomization to the earliest date of discontinuation of randomized treatment, start of an alternative therapy, or death.

TABLE 21 shows OS, PFS, and EFS median survival, Compound I-1/TC HR with 95% CI, and p values for the primary ITT population as well as for patients who received at least 4 cycles (N=476 patients), and those who received at least 6 cycles (N=375 patients).

TABLE 21
OS, PFS, and EFS results for ITT, and subgroups receiving at least 4 or 6 cycles

| ITT Population | | Patients with >4 | | Patients with >6 | |
|---|---|---|---|---|---|
| (n = 815) | | Cycles (n = 476) | | Cycles (n = 375) | |
| Compound I-1 | TC | Compound I-1 | TC | Compound I-1 | TC |

OS

| | | | | | | |
|---|---|---|---|---|---|---|
| Median | 7.1 | 8.5 | 15.6 | 13 | 19.5 | 15 |
| (months) | | | | | | |

| | | | |
|---|---|---|---|
| HR (95% CI), | 0.97 (0.83-1.14), p 0.73 | 0.78 (0.64-0.96), p 0.02 | 0.69 (0.54-0.88), p 0.002 |
| log rank p value | | | |

PFS

| | | | | | | |
|---|---|---|---|---|---|---|
| Median | 5.3 | 5.5 | 10.3 | 8.7 | 11.9 | 10.5 |
| (months) | | | | | | |

| | | | |
|---|---|---|---|
| HR (95% CI), | 0.99 (0.86-1.15), p 0.93 | 0.88 (0.73-1.06), p 0.17 | 0.86 (0.7-1.07), p 0.17 |
| log rank p value | | | |

EFS

| | | | | | | |
|---|---|---|---|---|---|---|
| Median | 5.1 | 5.3 | 12.1 | 8.9 | 14.9 | 11.1 |
| (months) | | | | | | |

| | | | |
|---|---|---|---|
| HR (95% CI), | 0.85 (0.74-0.98), p 0.02 | 0.74 (0.61-0.9), p 0.002 | 0.69 (0.56-0.86), p 0.001 |
| log rank p value | | | |

Compound I-1/TC HR for all analyses favored Compound I-1 (HR<1). However only OS, and EFS seemed to favor Compound I-1 in patients who received adequate treatment duration by number of cycles. EFS was also the only analysis to favor Compound I-1 significantly in the overall ITT population. This result suggests that EFS may be a better predictor of OS benefit in patients who went on to receive adequate treatment with at least 4 or 6 cycles. In addition, EFS also significantly favored Compound I-1 in patients who achieved an objective response (CR, CRp, CRi, or PR): median EFS for Compound I-1 17.4 vs 14.6 m for TC, HR 0.68, 95% CI 0.5-0.93, p 0.016.

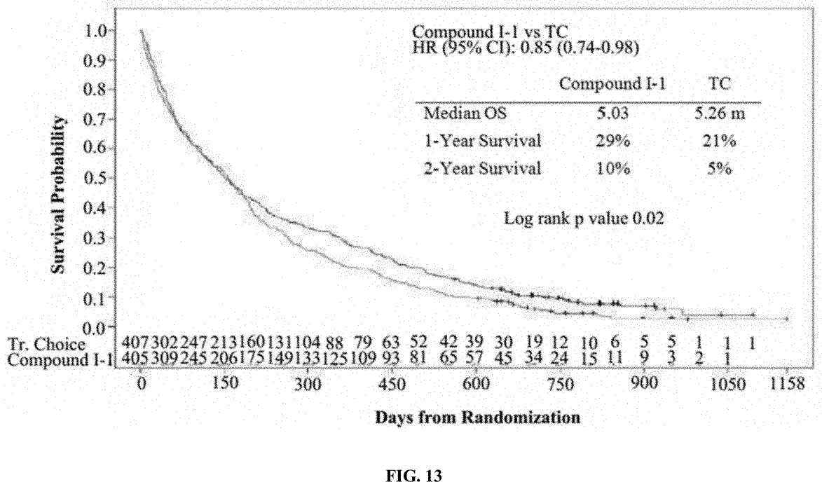

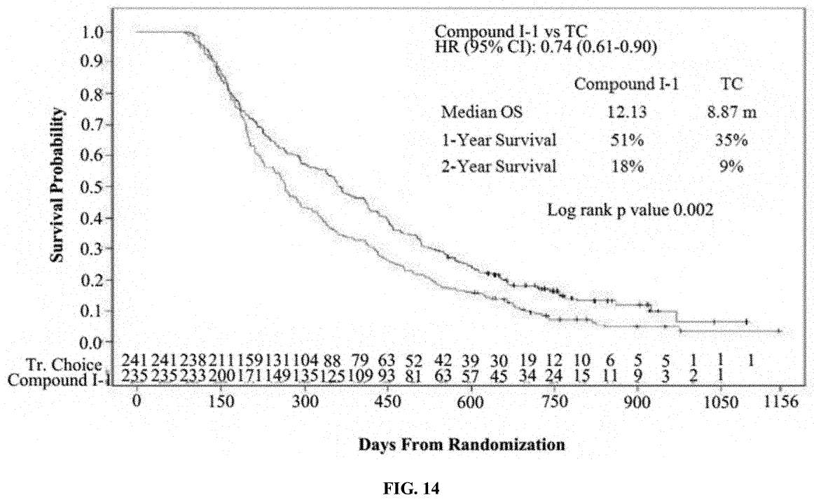

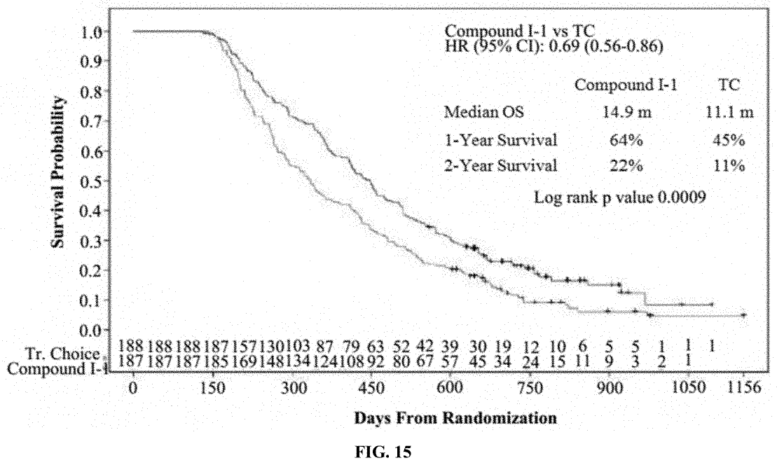

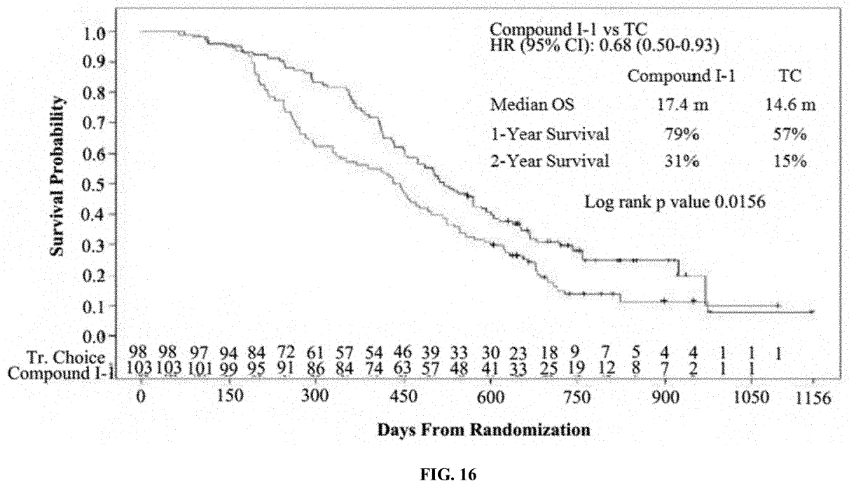

FIG. 13 provides a depiction of EFS in the primary ITT population (n=815). FIG. 14 shows the EFS in the subgroup that received at least 4 cycles of treatment (n=476). FIG. 15 demonstrates the EFS in the subgroup who received at least 6 cycles of treatment (n=375). FIG. 16 demonstrates the EFS in responders who had at least CRc or PR (n=201).

Conclusions:

EFS analyses that do not rely on progression date favored Compound I-1 over TC in the ITT population, and seemed to better predict OS benefit in patients who went on to receive at least 4 or 6 cycles.

### Example 8: Landmark Response and Survival Analyses from AML Patients Treated with Compound I-1 in a Phase 2 Study

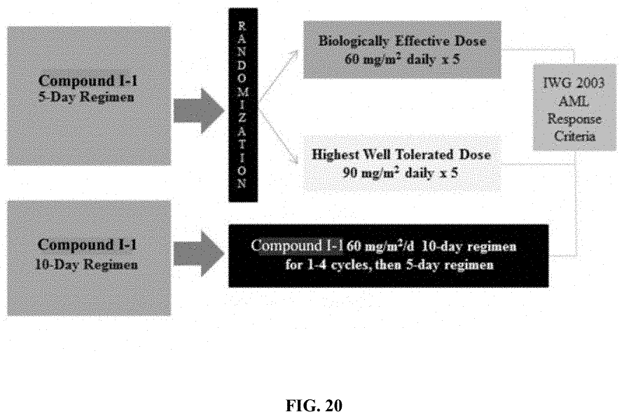

In a Phase 2 study of 206 AML patients, both treatment naïve (TN), and relapsed/refractory (r/r), patients were treated with Compound I-1 using different doses and schedules. The study design is shown in FIG. 20. The patients were randomized either to a 5- or 10-day dosing regimen. The patients who were part of the 10-day regimen were given Compound I-1 at 60 mg/m²/day for 1-4 cycles, then were given the 5-day regimen. The patients who were part of the 5-day regimen were randomized either to the biologically effective dose group (60 mg/m² daily) or the highest well tolerated dose group (90 mg/m² daily). The data set for the study is shown in TABLE 22 below.

TABLE 22

| | TN AML: | r/r AML: | Total AML: | |
|---|---|---|---|---|
| | (n = 1-3) | n = 103 | n = 206 | |
| Alive at 3 months¹ | 76 (74%) | 85 (83%) | 161 | (78%) |
| Discontinued (<4 cycles)³ | 17 (22%) | 47 (55%) | 64 | (40%) |
| Continued (>4 cycles)³ | 59 (78%) | 38 (45%) | 97 | (60%) |
| Alive at 5 months² | 66 (64%) | 67 (65%) | 133 | (65%) |
| Discontinued (<6 cycles)⁴ | 26 (39%) | 56 (84%) | 82 | (62%) |
| Continued (>6 cycles)⁴ | 40 (61%) | 11 (16%) | 51 | (38%) |

¹Data Set for Landmark Analysis at 4 Cycles
²Data Set for Landmark Analysis at 6 cycles
³% Calculated from the dataset alive at 3 months
⁴% calculated for dataset alive at 5 months

Methods:

Landmark response (CR, CRi, or CRp based on 2003 IWG criteria, grouped together as composite CR or CRc), and overall survival (OS) analyses for patients alive at or beyond month 3 and month 5 (time of planned start of cycle 4 and cycle 6, respectively) were conducted. Landmark OS was compared between patients who received at least 4 or 6 cycles and those patients who did not receive at least 4 or 6 cycles of treatment. The landmark methodology avoided the bias of early deaths before cycles 4 and 6 attributing a survival benefit in those who did not die early and were able to get more cycles. The results in responding and non-responding patients were also compared to see whether survival benefit was restricted to responding patients only.

Results:

The study completed enrolment with 206 AML patients: 103 patients (50%) for each of Treatment Naïve (TN) unfit for intensive chemotherapy, and relapsed/refractory (r/r) AML. Median age was 68.5y (range 22-92y), ECOG PS ≥2 in 26%, poor risk cytogenetics in 41%, secondary AML in 26%, and median baseline BM blasts % was 40% in the total AML population. 108 patients (52.4%), and 155 patients (75%) received <4 and <6 cycles respectively. TABLE 23 below provides a summary of the baseline characteristics for the patients in the study.

TABLE 23
Baseline Characteristics of Patients

| | Alive at 3 Months | | | | Alive at 5 Months | | | |
|---|---|---|---|---|---|---|---|---|
| | (n = 161) | | | | (n = 133) | | | |
| | <4 cycles | | >4 cycles | | <6 cycles | | >6 cycles | |
| | (n = 64) | | (n = 97) | | (n = 82) | | (n = 51) | |
| Median Age (range) | 67 | (22-92) | 76 | (33-85) | 67 | (22-85) | 77 | (49-85) |
| years | | | | | | | | |
| Sex M/F % | 59%/41% | | 55%/45% | | 56%/44% | | 53%/47% | |
| ECOG PS 0-1/2-3% | 80%/20% | | 77%/23% | | 83%/17% | | 75%/25% | |
| BM Blasts % Median | 38% | (4-94) | 39% | (7-95) | 33% | (4-95) | 40% | (9-90) |
| (range) | | | | | | | | |

| | | | | |
|---|---|---|---|---|
| WBCs >20,000/μL | 9% | 8% | 4% | 14% |
| Poor Risk | 38% | 36% | 33% | 37% |
| Cytogenetics | | | | |
| Secondary AML | 25% | 30% | 24% | 31% |

The primary reasons for treatment discontinuation before 4 and 6 cycles, respectively, (% from the total population of 206 patients) were early progression (20.4, and 30.6%), and early death (12.6%, and 17%). However, 9.7% and 14% discontinued treatment before 4 or 6 cycles, respectively, as a result other less objective reasons such as patient decision, investigator decision, or adverse events that might have been manageable without treatment discontinuation. TABLE 24 provides details for treatment discontinuation.

TABLE 24
Primary Reasons for Treatment Discontinuation

| | Alive at 3 months | Alive at 5 months |
|---|---|---|
| | (n = 161) | (n = 133) |
| Treatment Discontinuation | <4 cycles n = 64 | <6 cycles n = 82 |
| Progressive Disease | 48% | 48% |
| Patient or Investigator | 16% | 16% |
| Decision | | |
| Death | 3% | 6% |
| Adverse Events | 5% | 2% |
| Other | 5% | 2% |

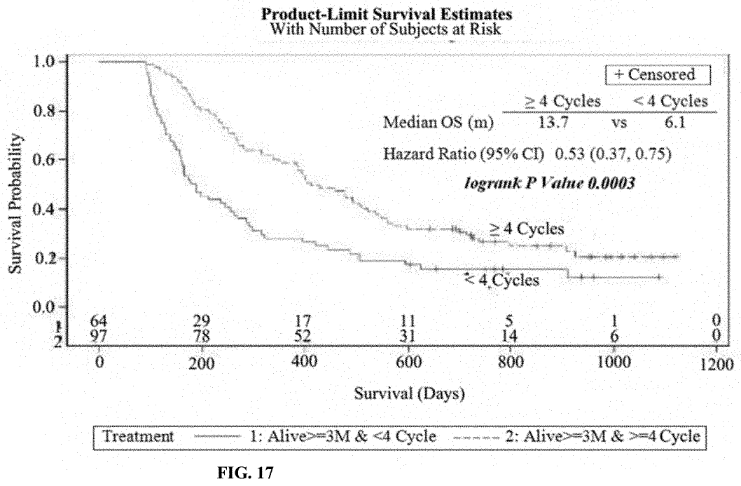

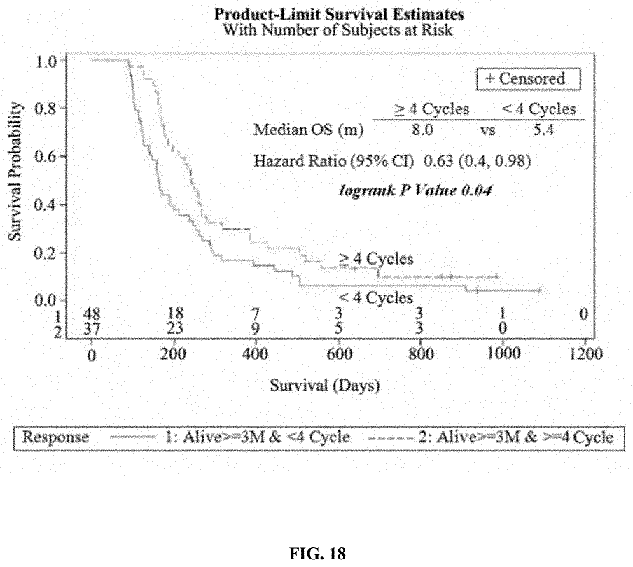

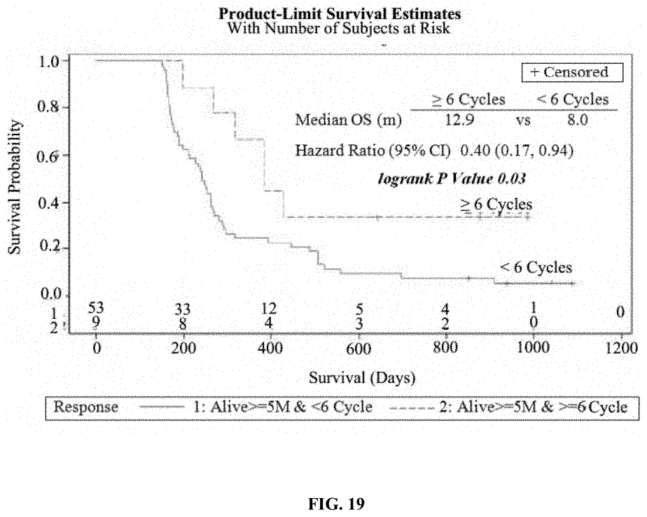

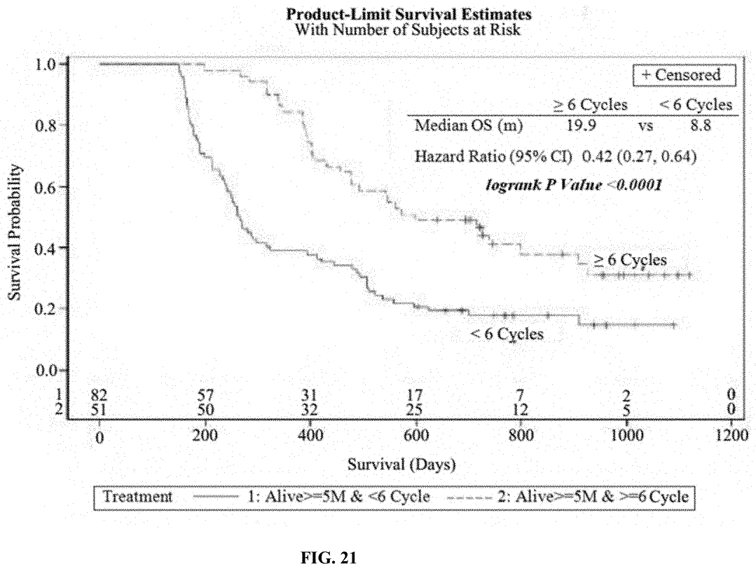

The landmark analysis population included 161 patients for 4-cycle analysis, and 133 for the 6-cycle analysis. In those patients, there were no major differences in baseline characteristics between those who received at least 4 or 6 cycles and those who did not. In the landmark analysis comparing those who received at least 4 cycles (97 patients) and those who did not (64 patients), CRc rate was 62% vs 25% (p<0.0001) and median OS was 13.7 m vs 6.1 m, respectively (HR 0.53, 95% CI 0.37-0.75, p 0.0003; FIG. 17). In the landmark analysis comparing those who received at least 6 cycles (51 patients) and those who did not (82 patients), the CRc rate was 82.4% vs 35.4% (p<0.0001), and median OS of 19.9 m vs 8.8 m, respectively (HR 0.42, 95% CI 0.27-0.64, p<0.0001; FIG. 21). The results were consistent when TN and r/r AML patients were analyzed separately. The landmark OS benefit in patients who received at least 4 or 6 cycles was also observed in patients who did not have an objective CRc. Patients with no CRc who received at least 4 cycles had a median OS of 8 m vs 5.4 m in those who did not (HR 0.63, 95% CI 0.40-0.98, p 0.04; FIG. 18). Patients with no CRc who received at least 6 cycles had a median OS of 12.9 m vs 8 m in those who did not (HR 0.40, 95% CI 0.17-0.94, p 0.03; FIG. 19).

Summary:

In a prospective phase 2 study of 206 TN and r/r AML patients treated with the HMA Compound I-1, patients who were alive at or beyond 3 and 5 months who continued treatment for at least 4 or 6 cycles, respectively, achieved a highly significant response and survival benefit compared to those who discontinued treatment before cycle 4 or 6. The survival benefit was observed even in patients who did not have an objective response.

### Example 9: Analysis of Relapsed/Refractory AML Patients

A Phase 2 study of Compound I-1 using different regimens and doses (randomized 5-day regimen cohorts of 60 mg/m²/d vs 90 mg/m²/d SC) and a cohort of 10-day regimen in the first 1-4 cycles at 60 mg/m²/d followed by subsequent cycles of 5-day regimen) was conducted as described above. Response and duration of response were assessed using IWG 2003 criteria: Complete Response (CR), CR with incomplete platelet recovery (CRp), and CR with incomplete count recovery (CRi). CR+CRp+CRi was defined as composite CR (CRc). Overall survival (OS) was assessed using the Kaplan-Meier (KM) method. Response status for each dose/regimen cohort and the overall treated population were assessed with analyses of duration of response and long-term survival.

Results:

The study completed enrollment of 103 r/r AML patients: 50 patients received the 5-day regimen at 60 mg/m²/d (24 patients) or 90 mg/m²/d (26 patients), and 53 patients received the 10-day regimen (60 mg/m²/d). Median follow up was 2.4 years (29.1 months). Patients' characteristics for the 103 r/r AML patients enrolled included median age of 60y (range 22-82y), poor risk cytogenetics in 41% of patients, prior hematopoietic cell transplant (HCT) in 18% of patients, median number of prior regimens 2 (range 1-10), primary refractory to induction therapy in 47%, and 41% had a high disease burden of BM blasts >40%. TABLE 25 provides details of the patients' baseline characteristics.

TABLE 25
Baseline Characteristics of Patients

| Characteristic | | N (%) | |
|---|---|---|---|
| Age (y) | Median [range] | 60 | [22-82] |
| | (>60 years) | 52 | (50%) |
| ECOG PS 0-1 | | 89 | (86%) |
| Cytogenetics | | | |
| Poor | | 42 | (41%) |
| Intermediate | Diploid | 21 | (20%) |
| | Miscellaneous | 31 | (30%) |
| Prior HCT | | 19 | (18%) |
| Prior Number of Rx Regimens | Median (range) | 2 | (1-10) |
| | 1 | 27 | (26%) |
| | 2 | 31 | (30%) |
| | 3-5 | 39 | (38%) |
| | >5 | 2 | (2%) |
| Response to First Induction | CR | 55 | (53%) |
| | Primary Refractory | 48 | (47%) |
| Baseline BM Blasts | Median (Range) | 33% | (2-95%) |
| | >40% | 42 | (41%) |

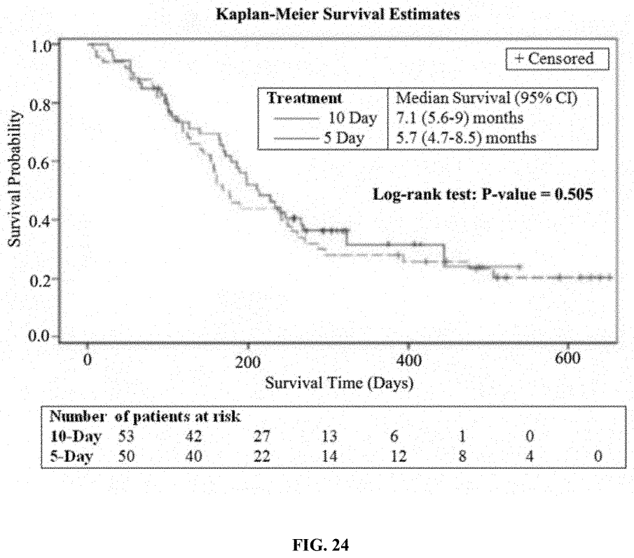

No significant difference was observed in CR or OS between 60 and 90 mg/m²/d 5-day regimen but the CR and CRc rates were higher on the 10-day regimen (19% and 30% respectively) vs the 5-day regimen (8% and 16%), as detailed in TABLE 26 and FIG. 24.

TABLE 26
Compound I-1 in r/r AML Clinical Responses

| | | | |
|---|---|---|---|
| | Response Rate | Response Rate | |
| | (n = 50) | (n = 53) | |
| | 5 Day | 10 Day | |
| Response | (60 and 90 mg/m²) | (60 mg/m²) | P |
| Category | N (%) | N (%) | value |

| | | | | | |
|---|---|---|---|---|---|
| CR | 3 | (6%) | 10 | (19%) | 0.074 |
| CRp | 1 | (2%) | 4 | (7%) | |
| CRi | 4 | (8%) | 2 | (4%) | |
| CRc | 8 | (16%) | 16 | (30%) | 0.106 |

| | | |
|---|---|---|
| (CR + | (95% CI: | (95% CI: |
| CRp + CRi) | 7, 29%) | 18, 44%) |

When all regimens were analyzed together, 24/103 patients (23.3%) achieved CRc. CRc responses were achieved in several poor prognosis subgroups including 19% in patients with poor risk cytogenetics, 31% of refractory patients, 26% of patients who relapsed after prior HCT, and 22% in patients with early relapse (<6 months from their prior treatment), as detailed in TABLE 27.

TABLE 27
Compound I-1 in r/r AML Clinical
Response (CRc) in Major Subgroups

| | | | | CRc | |
|---|---|---|---|---|---|
| Characteristic | Category | N (%) | | N (%) | |
| Age (y) | <65 | 63 | (61%) | 12 | (19%) |
| | >65 | 40 | (39%) | 12 | (30%) |
| ECOG PS | 0-1 | 89 | (86%) | 23 | (26%) |
| | 2 | 12 | (14%) | 1 | (7%) |
| Cytogenetics | Adverse | 42 | (41%) | 8 | (19%) |
| | Diploid | 21 | (20%) | 4 | (19%) |
| | Others | 40 | (39%) | 12 | (30%) |
| Prior HCT | Yes | 19 | (18%) | 5 | (26%) |
| | No | 84 | (82%) | 19 | (23%) |
| Response to Induction | Refractory | 48 | (47%) | 12 | (25%) |
| | CR | 55 | (53%) | 9 | (15%) |
| Time From Last Therapy | <6 months | 82 | (80%) | 18 | (22%) |
| | >6 months | 20 | (19%) | 6 | (30%) |

Of the 48 patients who were refractory to induction, 12 achieved CRc (25%). Of the 19 patients who had prior HCT, 5 (26% achieved CRc).

Median overall duration of response for patients with CR, and CRc were 7 and 7.8 months, respectively, as shown in TABLE 28 below.

TABLE 28
Follow Up, Treatment Exposure, and Duration of Response

| | Median (Range) | |
|---|---|---|
| Follow Up | 29.2 (25-32) | Months |
| Number of Cycles | 3 (1-29) | Cycles |
| Complete Response (CR) | 7 (1-29.5) | Months |
| Duration of Response | | |
| CR Composite | 7.8 (0.5-30) | Months |
| (CR + CRp + CRi) | | |
| Duration of Response | | |

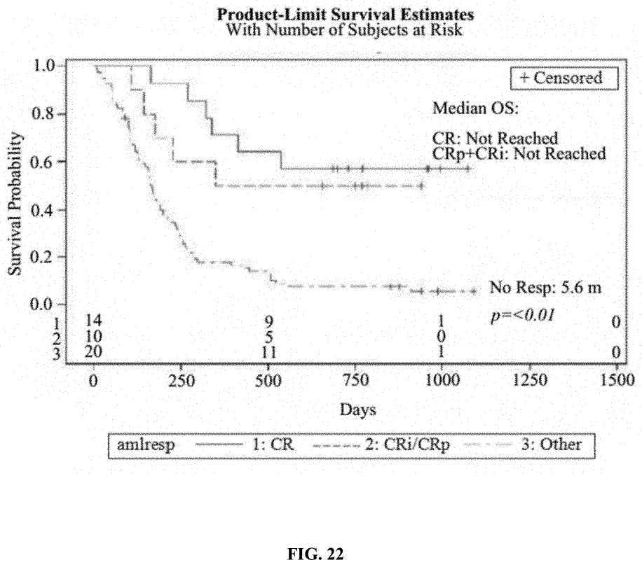

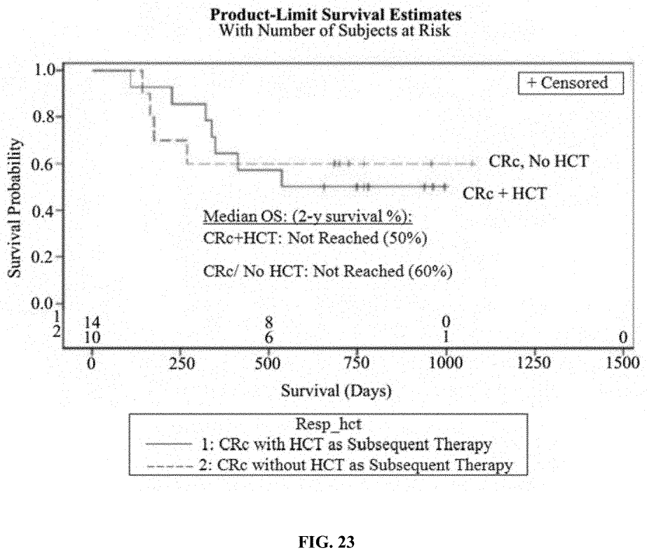

After long term follow up, median OS has not been reached in patients who achieved CRc (either CR or CRp/CRi). The 2-year survival rate was 57% for CR, and 50% for CRp/CRi (FIG. 22). Median OS had not yet been reached and was similar in CRc patients who went on to receive HCT post CRc (14 patients) compared to CRc patients who did not receive HCT post treatment (10 patients) (FIG. 23). The 2-year survival rate was also similar for both groups (50% for those receiving HCT vs 60% for those who did not undergo HCT). Most patients were still on Compound I-1 treatment until death, progression, or HCT with no other subsequent treatment. Compound I-1 was well tolerated in all cohorts with Grade 3 or higher AEs related to the drug. Grade 3 or higher AEs that were seen in 42% of patients included predominantly myelosuppression and related infections. There was no related serious AEs leading to death. The major adverse events were febrile neutropenia (60%; 62 patients); pneumonia (36%; 37 patients); thrombocytopenia (36%; 37 patients); anemia (31%; 32 patients); neutropenia (19%; 20 patients), or sepsis (16%; 16 patients). The all-cause early mortality was 3.9% (4 patients) within 30 days, and 11.7% (12 patients) within 60 days.

The results demonstrate the long survival benefit for Compound I-1 responders that exceeds duration of response and seems irrespective of post treatment HCT. The results also suggest that in r/r AML patients treated with Compound I-1, CRp/CRi seem to confer a similar survival benefit to CR patients suggesting that the incomplete peripheral blood count recovery may reflect continued treatment-related myelosuppression rather than active residual disease.

Summary:

In a phase 2 study of HMA Compound I-1 in heavily pretreated r/r AML patients, 47% of whom had refractory disease, CR, CRp, and CRi all conferred long survival benefit. With a median follow up of almost 2.5 years, more than half of responding patients were still alive at 2 years and their median OS has not yet been reached. In addition, treatment with Compound I-1 allowed post treatment HCT in 58% of responders.

### Embodiments

### Embodiment 1

A method of treating cancer in a subject in need thereof, the method comprising: (a) administering to the subject a therapeutic regimen, wherein the therapeutic regimen comprises administration of a therapeutically-effective amount of a DNA-hypomethylating agent once per day on days 1-5 of a treatment cycle, wherein the treatment cycle lasts 28 days; and (b) repeating the therapeutic regimen at least 3 times.

### Embodiment 2

The method of embodiment 1, wherein the DNA-hypomethylating agent is a compound of Formula I or a pharmaceutically-acceptable salt thereof: (5-azacytosine group)-L-(guanine group) (I).

### Embodiment 3

The method of embodiment 2, wherein L is a phosphorous-containing linker wherein the number of phosphorous atoms in L is 1.

### Embodiment 4

The method of any one of embodiments 2-3, wherein in the compound of Formula I, L is of Formula (II):

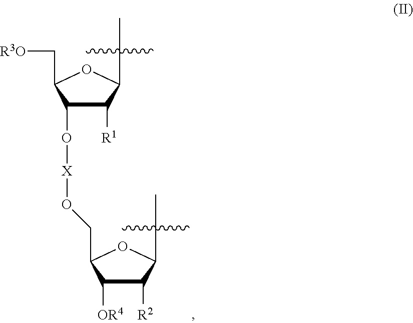

wherein, R¹ and R² are independently H, OH, an alkoxy group, an alkoxyalkoxy group, an acyloxy group, a carbonate group, a carbamate group, or a halogen; R³ is H, or R³ together with the oxygen atom to which R³ is bound forms an ether, an ester, a carbonate, or a carbamate; R⁴ is H, or R⁴ together with the oxygen atom to which R⁴ is bound forms an ether, an ester, a carbonate, or a carbamate; and X together with the oxygen atoms to which X is bound forms a phosphodiester, a phosphorothioate diester, a boranophosphate diester, or a methylphosphonate diester.

### Embodiment 5

The method of embodiment 4, wherein R¹ and R² are independently H, OH, OMe, OEt, OCH₂CH₂OMe, OBn, or F.

### Embodiment 6

The method of any one of embodiments 4-5, wherein X together with the oxygen atoms to which X is bound forms a phosphodiester.

### Embodiment 7

The method of any one of embodiments 4-6, wherein R¹ and R² are H.

### Embodiment 8

The method of any one of embodiments 2-7, wherein the compound of Formula I is Compound I-I:

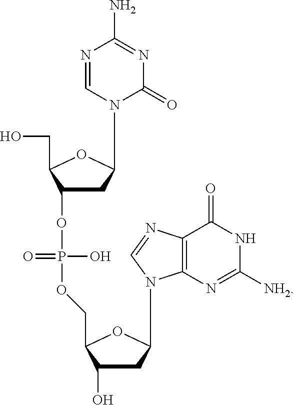

### Embodiment 9

The method of of any one of embodiments 2-7, wherein the compound of Formula I is Compound 1-2:

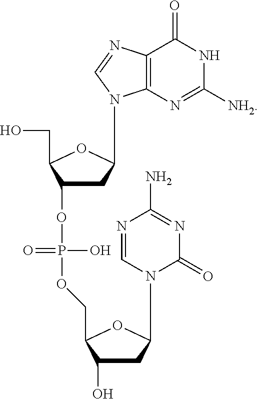

### Embodiment 10

The method of any one of embodiments 1-9, wherein the therapeutic regimen is repeated at least 6 times.

### Embodiment 11

The method of any one of embodiments 1-10, wherein the therapeutic regimen is more likely to prolong survival in the subject when the therapeutic regimen is administered to the subject at least 4 times compared to when the therapeutic regimen is administered to the subject less than 4 times.

### Embodiment 12

The method of any one of embodiments 1-11, wherein the therapeutic regimen is more likely to prolong survival in the subject by at least about 1 month when the therapeutic regimen is administered to the subject at least 4 times compared to when the therapeutic regimen is administered to the subject less than 4 times.

### Embodiment 13

The method of any one of embodiments 1-12, wherein the therapeutically-effective amount of the DNA hypomethylating agent is about 1 mg per m² of body surface area of the subject to about 200 mg per m² of body surface area of the subject.

### Embodiment 14

The method of any one of embodiments 1-12, wherein the therapeutically-effective amount of the DNA hypomethylating agent is about 60 mg per m² of body surface area of the subject.

### Embodiment 15

The method of any one of embodiments 1-12, wherein the therapeutically-effective amount of the DNA hypomethylating agent is about 90 mg per m² of body surface area of the subject.

### Embodiment 16

The method of any one of embodiments 1-15, wherein the administering is subcutaneous.

### Embodiment 17

The method of any one of embodiments 1-15, wherein the administering is intravenous.

### Embodiment 18

The method of any one of embodiments 1-17, wherein the cancer is a myelodysplastic syndrome.

### Embodiment 19

The method of any one of embodiments 1-17, wherein the cancer is acute myeloid leukemia.

### Embodiment 20

The method of any one of embodiments 1-17, wherein the cancer is acute promyelocytic leukemia.

### Embodiment 21

The method of any one of embodiments 1-17, wherein the cancer is acute lymphoblastic leukemia.

### Embodiment 22

The method of any one of embodiments 1-17, wherein the cancer is chronic myelogenous leukemia.

### Embodiment 23

The method of any one of embodiments 1-22, wherein after the administering, an enzymatic degradation of the DNA-hypomethylating agent within the subject produces a metabolite of the DNA-hypomethylating agent in the subject.

### Embodiment 24

The method of embodiment 23, wherein the metabolite of the DNA-hypomethylating agent is decitabine.

### Embodiment 25

The method of any one of embodiments 23-24, wherein the enzymatic degradation of the DNA-hypomethylating agent comprises:—enzymatic cleavage of a phosphodiester bond of the DNA-hypomethylating agent; and—enzymatic conversion of the metabolite of the DNA-hypomethylating agent into an active form of the metabolite within the subject.

### Embodiment 26

The method of embodiment 25, wherein enzymatic conversion of the metabolite of the DNA-hypomethylating agent into the active form of the metabolite is mediated by deoxycytidine kinase.

### Embodiment 27

The method of any one of embodiments 25-26, wherein the active form of the metabolite is incorporated into replicating DNA.

### Embodiments 28

The method of any one of embodiments 23-27, wherein administration of the DNA-hypomethylating agent provides a blood circulation time of the metabolite in the subject that is greater than a blood circulation time of the metabolite that results from administration of the metabolite to the subject.

### Embodiment 29

The method of any one of embodiments 1-28, wherein a likelihood of the subject experiencing an adverse event associated with the DNA-hypomethylating agent is decreased when the therapeutically-effective amount of the DNA-hypomethylating agent is about 60 mg per m² of body surface area of the subject compared to when the therapeutically-effective amount of the DNA-hypomethylating agent is about 90 mg per m² of body surface area of the subject.

### Embodiment 30

The method of any one of embodiments 1-29, wherein global DNA demethylation in the subject is decreased when the therapeutically-effective amount of the DNA-hypomethylating agent is about 60 mg per m² of body surface area of the subject compared to when the therapeutically-effective amount of the DNA-hypomethylating agent is about 90 mg per m² of body surface area of the subject.

### Embodiment 31

The method of embodiment 29, wherein the adverse event is at least a grade 3 adverse event.

### Embodiment 32

The method of any one of embodiments 1-31, wherein the administration is no more than once per day on days 1-5 of a treatment cycle.

### Embodiment 33

The method of any one of embodiments 1-32, wherein the DNA-hypomethylating agent is not administered on days 6-28 of the treatment cycle.

### Embodiment 34

A method of treating cancer in a subject in need thereof, the method comprising administering to the subject a therapeutically effective amount of a DNA-hypomethylating agent on days 1-5 of a 28-day treatment cycle, wherein the therapeutically effective amount of the DNA-hypomethylating agent is about 60 mg per m² of body surface area of the subject, and wherein, in a controlled study:—each human of a group of humans has cancer;—60 mg per m² of body surface area of the DNA-hypomethylating agent is administered to each human of the group of humans on days 1-5 of a 28-day study treatment cycle; and—the group of humans has a median survival of about 408 to about 771 days.

### Embodiment 35

A method of treating cancer in a subject in need thereof, the method comprising administering to the a therapeutically effective amount of a DNA-hypomethylating agent on days 1-5 of a 28-day treatment cycle, wherein the therapeutically effective amount of the DNA-hypomethylating agent is about 90 mg per m² of body surface area of the subject, and wherein, in a controlled study:—each human of a group of humans has cancer;—90 mg per m² of body surface area of the DNA-hypomethylating agent is administered to each human of the group of humans on days 1-5 of a 28-day study treatment cycle; and—the group of humans has a median survival of about 303 to about 663 days.

### Embodiment 36

A method of treating cancer in a subject in need thereof, the method comprising administering to the subject a therapeutically effective amount of a DNA-hypomethylating agent on days 1-5 of a 28-day treatment cycle, wherein the therapeutically effective amount of the DNA-hypomethylating agent is about 60 mg per m² of body surface area of the subject, and wherein, in a controlled study of a group of humans:—each human of the group of humans has cancer;—60 mg per m² of body surface area of the DNA-hypomethylating agent is administered to each human of the group of humans on days 1-5 of a 28-day study treatment cycle;—about 52% to about 78% of humans of the group of humans survive for at least 12 months after day 1 of the 28-day study treatment cycle; and—about 26% to about 52% of humans of the group of humans survive for at least 24 months after day 1 of the 28-day study treatment cycle.

### Embodiment 37

A method of treating cancer in a subject in need thereof, the method comprising administering to the subject a therapeutically effective amount of a DNA-hypomethylating agent on days 1-5 of a 28-day treatment cycle, wherein the therapeutically effective amount of the DNA-hypomethylating agent is about 90 mg per m² of body surface area of the subject, and wherein, in a controlled study:—each human of a group of humans has cancer;—90 mg per m² of body surface area of the DNA-hypomethylating agent is administered to each human of the group of humans on days 1-5 of a 28-day study treatment cycle;—about 45% to about 72% of humans of the group of humans survive for at least 12 months after day 1 of the 28-day study treatment cycle; and—about 18% to about 43% of humans of the group of humans survive for at least 24 months after day 1 of the 28-day study treatment cycle.

1. A method of treating cancer in a subject in need thereof, the method comprising:
(a) administering to the subject a therapeutic regimen, wherein the therapeutic regimen comprises administration of a therapeutically-effective amount of a DNA-hypomethylating agent once per day on days 1-5 of a treatment cycle, wherein the treatment cycle lasts 28 days; and
(b) repeating the therapeutic regimen at least 3 times.

2. The method of claim 1, wherein the DNA-hypomethylating agent is a compound of Formula I or a pharmaceutically-acceptable salt thereof:
(5-azacytosine group)-L-(guanine group) (I)

3. The method of claim 2, wherein L is a phosphorous-containing linker wherein the number of phosphorous atoms in L is 1.

4. The method of claim 2, wherein in the compound of Formula I, L is of Formula (II): 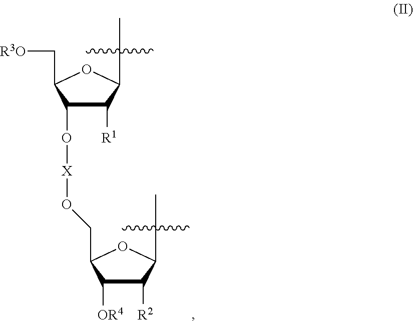
wherein, R¹ and R² are independently H, OH, an alkoxy group, an alkoxyalkoxy group, an acyloxy group, a carbonate group, a carbamate group, or a halogen; R³ is H, or R³ together with the oxygen atom to which R³ is bound forms an ether, an ester, a carbonate, or a carbamate; R⁴ is H, or R⁴ together with the oxygen atom to which R⁴ is bound forms an ether, an ester, a carbonate, or a carbamate; and X together with the oxygen atoms to which X is bound forms a phosphodiester, a phosphorothioate diester, a boranophosphate diester, or a methylphosphonate diester.

5-7. (canceled)

8. The method of claim 2, wherein the compound of Formula I is Compound I-1:

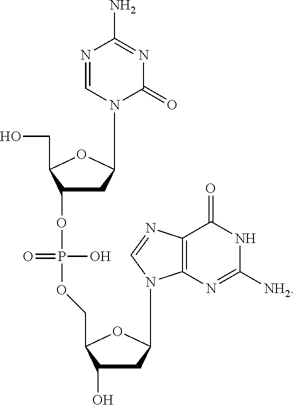

9. The method of claim 2, wherein the compound of Formula I is Compound 1-2:

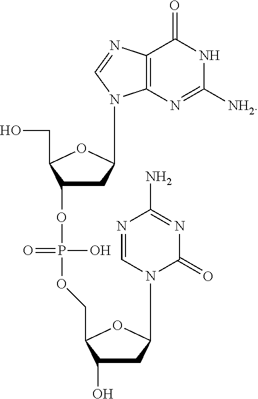

10. The method of claim 1, wherein the therapeutic regimen is repeated at least 6 times.

11. The method of claim 1, wherein the therapeutic regimen is more likely to prolong survival in the subject when the therapeutic regimen is administered to the subject at least 4 times compared to when the therapeutic regimen is administered to the subject less than 4 times.

12. The method of claim 1, wherein the therapeutic regimen is more likely to prolong survival in the subject by at least about 1 month when the therapeutic regimen is administered to the subject at least 4 times compared to when the therapeutic regimen is administered to the subject less than 4 times.

13. The method of claim 1, wherein the therapeutically-effective amount of the DNA hypomethylating agent is about 1 mg per m² of body surface area of the subject to about 200 mg per m² of body surface area of the subject.

14. The method of claim 1, wherein the therapeutically-effective amount of the DNA hypomethylating agent is about 60 mg per m² of body surface area of the subject.

15. The method of claim 1, wherein the therapeutically-effective amount of the DNA hypomethylating agent is about 90 mg per m² of body surface area of the subject.

16. The method of claim 1, wherein the administering is subcutaneous.

17. The method of claim 1, wherein the administering is intravenous.

18. The method of claim 1, wherein the cancer is a myelodysplastic syndrome.

19. The method of claim 1, wherein the cancer is acute myeloid leukemia.

20. The method of claim 1, wherein the cancer is acute promyelocytic leukemia.

21. The method of claim 1, wherein the cancer is acute lymphoblastic leukemia.

22. The method of claim 1, wherein the cancer is chronic myelogenous leukemia.

23. The method of claim 1, wherein after the administering, an enzymatic degradation of the DNA-hypomethylating agent within the subject produces a metabolite of the DNA-hypomethylating agent in the subject.

24. The method of claim 23, wherein the metabolite of the DNA-hypomethylating agent is decitabine.

25. The method of claim 23, wherein the enzymatic degradation of the DNA-hypomethylating agent comprises:
enzymatic cleavage of a phosphodiester bond of the DNA-hypomethylating agent; and
enzymatic conversion of the metabolite of the DNA-hypomethylating agent into an active form of the metabolite within the subject.

26. The method of claim 25, wherein enzymatic conversion of the metabolite of the DNA-hypomethylating agent into the active form of the metabolite is mediated by deoxycytidine kinase.

27. The method of claim 25, wherein the active form of the metabolite is incorporated into replicating DNA.

28. The method of claim 23, wherein administration of the DNA-hypomethylating agent provides a blood circulation time of the metabolite in the subject that is greater than a blood circulation time of the metabolite that results from administration of the metabolite to the subject.

29. The method of claim 1, wherein a likelihood of the subject experiencing an adverse event associated with the DNA-hypomethylating agent is decreased when the therapeutically-effective amount of the DNA-hypomethylating agent is about 60 mg per m² of body surface area of the subject compared to when the therapeutically-effective amount of the DNA-hypomethylating agent is about 90 mg per m² of body surface area of the subject.

30. The method of claim 1, wherein global DNA demethylation in the subject is decreased when the therapeutically-effective amount of the DNA-hypomethylating agent is about 60 mg per m² of body surface area of the subject compared to when the therapeutically-effective amount of the DNA-hypomethylating agent is about 90 mg per m² of body surface area of the subject.

31. The method of claim 29, wherein the adverse event is at least a grade 3 adverse event.

32. The method of claim 1, wherein the administration is no more than once per day on days 1-5 of a treatment cycle.

33. The method of claim 1, wherein the DNA-hypomethylating agent is not administered on days 6-28 of the treatment cycle.

34-37. (canceled)
